(19) United States
(12) Patent Application Publication
(10) Pub. No.: US 2022/0411461 A1
(43) Pub. Date: Dec. 29, 2022

(54) METHODS OF MAKING INCRETIN ANALOGS

(71) Applicant: ELI LILLY AND COMPANY, Indianapolis, IN (US)

(72) Inventors: Michael Eugene Kopach, Greenwood, IN (US); Yu Lu, Zionsville, IN (US); Sergey Vladimirovich Tsukanov, Indianapolis, IN (US); Timothy Donald White, Zionsville, IN (US); Ankur Jalan, Indianapolis, IN (US); Jinju James, Dublin (IE); Michael E. Kobierski, Greenwood, IN (US)

(21) Appl. No.: 17/633,631

(22) PCT Filed: Aug. 18, 2020
(86) PCT No.: PCT/US2020/046778

§ 371 Date: Feb. 8, 2022

Related U.S. Application Data
(60) Provisional application No. 62/888,756, filed on Aug. 19, 2019.

(51) Int. Cl. C07K 1/02; C07K 1/10; C07K 14/605

(52) U.S. Cl. CPC C07K 1/02; C07K 1/10; C07K 14/605

(57) ABSTRACT

Intermediate compounds are disclosed for making incretin analogs, or pharmaceutically acceptable salts thereof. In addition, methods are disclosed for making incretin analogs by coupling from two to four of the intermediate compounds herein via hybrid liquid solid phase synthesis or native chemical ligation.

The disclosure relates generally to biology, chemistry and medicine, and more particularly it relates to methods of synthesizing, via hybrid liquid solid phase synthesis (HLSPS), an incretin analog having activity at one or more of the glucose-dependent insulinotropic polypeptide (GIP), glucagon-like peptide-1 (GLP-1) and glucagon (GCG) receptors.

Over the past several decades, the prevalence of diabetes has continued to rise. Type 2 diabetes (T2DM) is the most common form of diabetes, accounting for about 90% of all diabetes. T2DM is characterized by high blood glucose levels caused by insulin resistance. The current standard of care for T2DM includes dieting and exercising, as well as treating with oral glucose-lowering therapeutics and/or injectable glucose-lowering therapeutics, including incretin-based therapies, such as GLP-1 receptor agonists, GIP/GLP-1 dual receptor agonists and even GIP/GLP-1/GCG (GGG) tri-receptor agonists.

Intl. Patent Application Publication Nos. WO 2019/125938 and 2019/125929 generally describe incretin analogs that act as GGG tri-receptor agonists and a method of synthesizing the same via standard solid phase peptide synthesis. See also, Intl. Patent Application Publication Nos. WO 2014/049610, 2015/067716, 2016/198624, 2017/116204, 2017/153575 and 2018/100135. Likewise, Intl. Patent Application Publication Nos. WO 2013/164483 and 2016/111971 describe compounds stated to have GLP-1 and GIP activity. Moreover, Intl. Patent Application Publication No. WO 2020/023386 describes peptides having GIP and GLP1 receptor agonist activity.

There is a need, however, for alternative methods of making such incretin analogs and intermediates thereof to enable pharmaceutically elegant production with commercially desired purity. Likewise, there is a need for efficient methods and stable intermediates to provide incretin analogs efficiently, with fewer purification steps.

To address this need, the disclosure describes methods of making incretin analogs via HLSPS or native chemical ligation (NCL), where such methods use from two to four intermediate compounds to make the incretin analog.

In a first embodiment, the incretin analog can include an amino acid sequence of: YX₂QGTFTSDYSIX₁₃LDKX₁₇AX₁₉X₂₀AFIEYLLX₂₈X₂₉GPSSX₃₄APPPS, where X₂ is Aib, X₁₃ is L or αMeL, X₁₇ is any amino acid with a functional group available for conjugation, and the functional group is conjugated to a C₁₆-C₂₂ fatty acid, X₁₉ is Q or A, X₂₀ is Aib, αMeK, Q or H, X₂₈ is E or A, X₂₉ is G or Aib, X₃₄ is G or Aib (SEQ ID NO:4) and the C-terminal amino acid is optionally amidated, or a pharmaceutically acceptable salt thereof. In certain instances, the incretin analog can have an amino acid sequence of: Y(Aib)QGTFTSDYSI(αMeL)LDKKAQ(Aib)AFIEYLLEGGPSSGAPPPS (SEQ ID NO:5), where the C-terminal amino acid is optionally amidated, or a pharmaceutically acceptable salt thereof.

In some instances, the C₁₆-C₂₂ fatty acid can be attached to the incretin analog via a linker having a structure of: (2-[2-(2-amino-ethoxy)-ethoxy]-acetyl)ₐ-(γGlu)_{b}-CO—(CH₂)_{c}—CO₂H, where a can be 0, 1 or 2, b can be 1 or 2, and c can be 16 or 18.

In particular instances, the incretin analog can have the following sequence: Y(Aib)QGTFTSDYSI(αMeL)LDKK((2-[2-(2-amino-ethoxy)-ethoxy]-acetyl)-(γGlu)-CO—(CH₂)₁₈—CO₂H)AQ(Aib)AFIEYLLEGGPSSGAPPPS-NH₂ (SEQ ID NO:6), or a pharmaceutically acceptable salt thereof, which can be depicted as having a structure of:

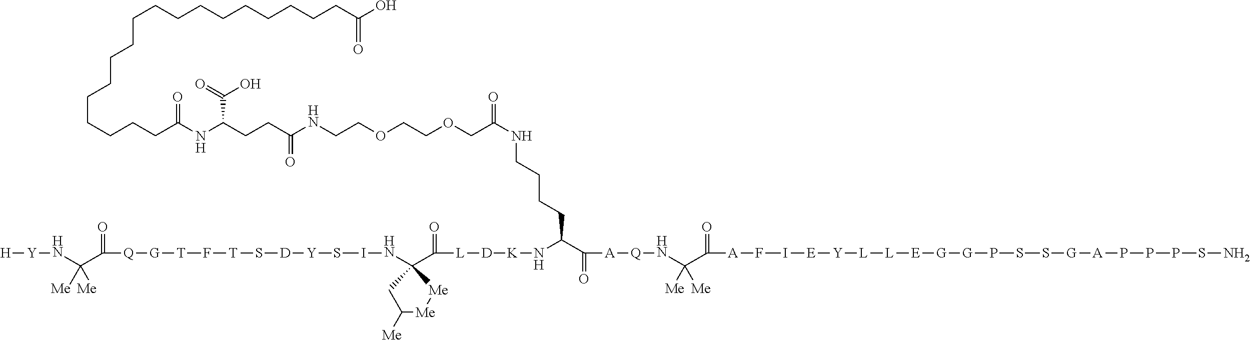

With regard to methods of making the incretin analog of SEQ ID NO:6 via HLSPS, the methods can include at least a step of coupling four intermediate compounds, where such compounds have a structure as recited in SEQ ID NOS:7, 8, 9 and 10, or pharmaceutically acceptable salts thereof.

Alternatively, the methods can include at least a step of coupling four intermediate compounds, where such compounds have a structure as recited in SEQ ID NOS:7, 11, 12 and 10, or pharmaceutically acceptable salts thereof.

Alternatively, the methods can include at least a step of coupling four intermediate compounds, where such compounds have a structure as recited in SEQ ID NOS:7, 13, 14 and 10, or pharmaceutically acceptable salts thereof.

In other instances, the methods can include at least a step of coupling three intermediate compounds, where such compounds have a structure as recited in SEQ ID NOS:7, 13 and 15, or pharmaceutically acceptable salts thereof.

Alternatively, the methods can include at least a step of coupling three intermediate compounds, where such compounds have a structure as recited in SEQ ID NOS:16, 17 and 10, or pharmaceutically acceptable salts thereof.

Alternatively, the methods can include at least a step of coupling three intermediate compounds, where such compounds have a structure as recited in SEQ ID NOS:18, 12 and 10, or pharmaceutically acceptable salts thereof.

Alternatively, the methods can include at least a step of coupling three intermediate compounds, where such compounds have a structure as recited in SEQ ID NOS:7, 45 and 10, or pharmaceutically acceptable salts thereof.

Alternatively, the methods can include at least a step of coupling three intermediate compounds, where such compounds have a structure as recited in SEQ ID NOS:7, 11 and 20, or pharmaceutically acceptable salts thereof.

In other instances, the methods can include at least a step of coupling two intermediate compounds, where such compounds have a structure as recited in SEQ ID NOS:19 and 15, or pharmaceutically acceptable salts thereof.

Alternatively, the methods can include at least a step of coupling two intermediate compounds, where such compounds have a structure as recited in SEQ ID NOS:18 and 20, or pharmaceutically acceptable salts thereof.

The methods above also can include a step of synthesizing the two to four intermediate compounds prior to the coupling step.

In the methods above, the intermediate compounds therefore can be chemically coupled or enzymatically coupled to one another to obtain the incretin analog of SEQ ID NO:6.

In the methods above, the C₁₆-C₂₂ fatty acid moiety and optional linker can be attached to one intermediate compound before the various intermediate compounds are coupled (i.e., acylation can occur before complete incretin analog synthesis). Alternatively, the fatty acid moiety can be attached to the incretin analog after the various intermediate compounds have been coupled (i.e., acylation can occur after complete incretin analog synthesis). For example, the methods can include at least a step of coupling two intermediate compounds, where such compounds have a structure as recited in SEQ ID NOS:21 and 18, or pharmaceutically acceptable salts thereof, followed by coupling of a fatty acid moiety having a structure of:

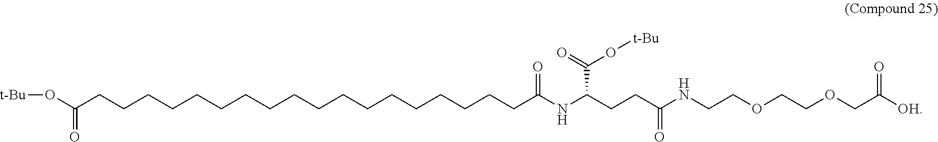 [SMILES: CC(C)(C)OC(=O)CCCCCCCCCCCCCCCCCCC(=O)N[C@@H](CCC(=O)NCCOCCOCC(=O)O)C(=O)OC(C)(C)C]

Alternatively, the methods can include at least a step of coupling the following two intermediate compounds, where such compounds have a structure as recited in SEQ ID NOS:22 and 19, followed by coupling of a fatty acid moiety having a structure of:

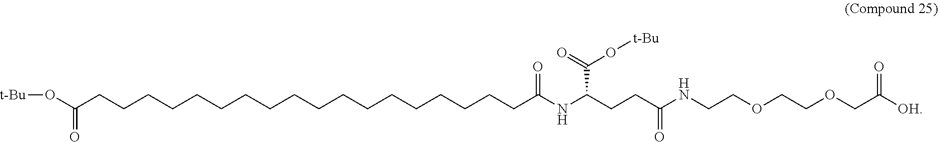 [SMILES: CC(C)(C)OC(=O)CCCCCCCCCCCCCCCCCCC(=O)N[C@@H](CCC(=O)NCCOCCOCC(=O)O)C(=O)OC(C)(C)C]

In addition to the above, one can alternatively use NCL to make an incretin analog of SEQ ID NO:6, in which the methods can include at least a step of coupling two intermediate compounds, where such compounds can have a structure selected from the following:
SEQ ID NOS:23 and 24,
SEQ ID NOS:39 and 24,
SEQ ID NOS:25 and 26,
SEQ ID NOS:40 and 26, and
SEQ ID NOS:27 and 26.

In another embodiment, the incretin analog can include an amino acid sequence of: Y(Aib)EGT(αMeF(2F))TSD(4Pal)SI(αMeL)LD(Orn)K((2-[2-(2-amino-ethoxy)-ethoxy]-acetyl)₂-(γ-Glu)-CO—(CH₂)₁₆—CO₂H)AQ(Aib)EFI(D-Glu)(αMeY)LIEGGPSSGAPPPS-NH₂ (SEQ ID NO:29), or a pharmaceutically acceptable salt thereof, which can be depicted as having a structure of:

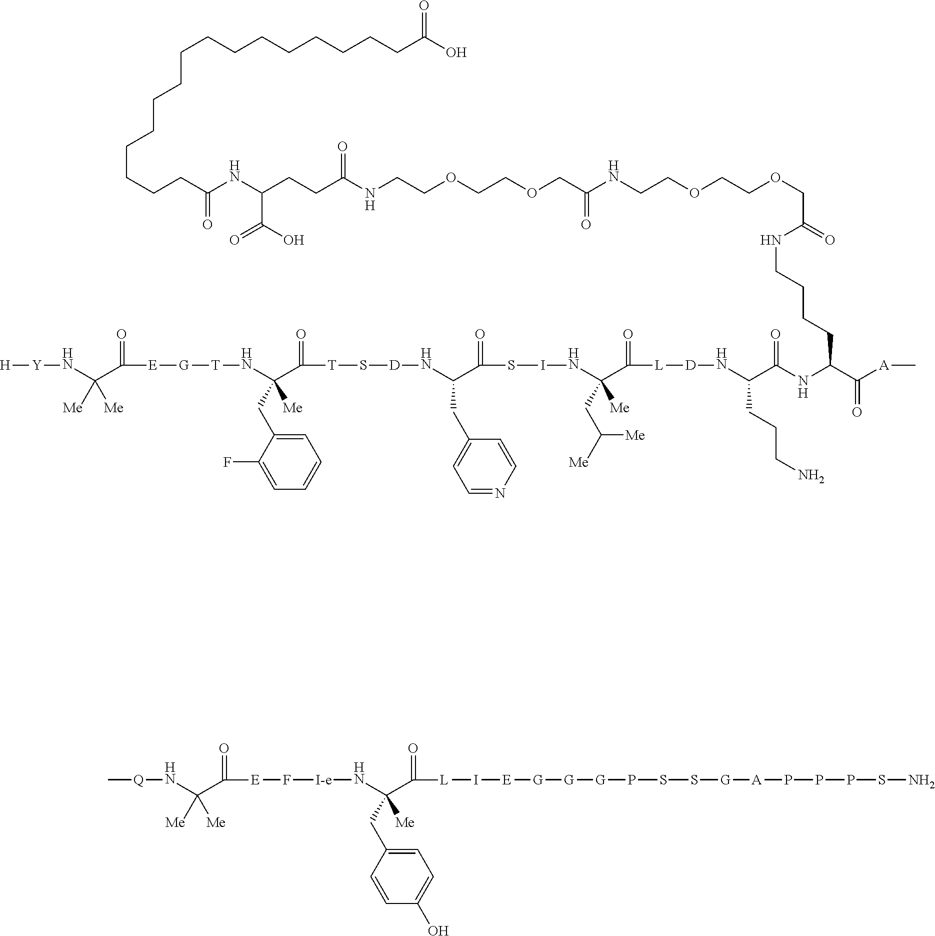

With regard to methods of making the incretin analog of SEQ ID NO:29 via HLSPS, the methods can include at least a step of coupling at least one of the following intermediate compounds to another intermediate compound, where such compounds have a structure as recited in SEQ ID NOS:30, 31, 32, 34, 35, 36 and/or 37, or pharmaceutically acceptable salts thereof.

Provided is a method of making an incretin analog of SEQ ID NO:29, the method comprising the step of:

coupling, via hybrid liquid solid phase synthesis, intermediate compounds selected from the groups consisting of:

a. SEQ ID NOS:7, 62, 42 and 31,

b. SEQ ID NOS:43, and 44.

In addition to the above methods, embodiments herein also include the intermediate compounds themselves (e.g., SEQ ID NOS:7 to 28 and 30 to 41), as well as compositions including the same.

An advantage of the analogs herein is that they can be used as effective treatments for diabetes mellitus, dyslipidemia, non-alcoholic fatty liver disease (NAFLD), metabolic syndrome, non-alcoholic steatohepatitis (NASH) and obesity, as well as other disorders or conditions associated with modulation of GLP-1, and/or GIP, and/or Glucagon.

An advantage of the methods herein includes several process improvements such as, for example, shorter fragments initially produced via SPPS allow for generally increased purity and higher yields via HLSPS.

An advantage of the methods herein includes that efficiency of the coupling in SPPS not only is dependent on the actual residues involved in the chemical transformation but also is impacted by structure attached to the resin (i.e., solubility/aggregation issues are well known for certain sequences). With shorter fragments, more route flexibility is available for couplings of complicated amino acid residues, and an ability to redesign fragment structures to address more difficult transformations.

An advantage of the methods herein includes an improved control strategy for impurities during the synthesis, which can enable an improved final impurity profile for the crude peptide and simplify/reduce chromatography burden resulting in the cost savings.

An advantage of the methods herein includes that synthesis of shorter fragments via SPPS can allow for reduced washing cycles, for reduced volumes of reagents, and for use of greener solvent(s) leading to a reduced process mass intensity (PMI).

An advantage of the methods herein includes that with shorter fragments, risks of failure typical in linear builds of a long molecule are significantly reduced.

An advantage of the methods herein includes that a combination of liquid and solid phase synthesis is more amenable to new manufacturing platforms and introducing other innovative technologies.

An advantage of the methods herein includes flexibility in supply chain and logistics of the manufacturing process by using several independent fragments.

An advantage of the methods herein includes that use parallel manufacturing of fragments can provide reduced manufacturing cycles by parallel processing of the fragments.

An advantage of the methods herein includes that current good manufacturing practice (cGMP) convergent steps can be executed at a standard facility without a need for specialized equipment.

Unless defined otherwise, all technical and scientific terms used herein have the same meaning as commonly understood by one of skill in the art to which the disclosure pertains. Although any methods and materials similar to or equivalent to those described herein can be used in the practice or testing of the incretin analog, pharmaceutical compositions and methods, the preferred methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

### Abbreviations and Definitions

Certain abbreviations are defined as follows: “AEEA” refers to 2-[2-(2-amino-ethoxy)-ethoxy]-acetyl, “D-Glu” or “e” refers to D-Glutamic acid, “e” in an amino acid sequence refers to D-Glutamic acid, “Aib” refers to α-amino isobutyric acid, “αMeL” refers to α-methyl leucine, “αMeK” refers to α-methyl lysine, “Boc” refers to tert-butoxycarbonyl, “Bu” refers to butyl, “t-Bu” refers to tert-Butyl, “CTC” refers to chlorotrityl chloride, “DCM” refers to dichloromethane, “DIC” refers to diisopropylcarbodiimide, “DMF” refers to dimethylformamide, “DMSO” refers to dimethyl sulfoxide, “DTT” refers to dithiothreitol, “EDTA” refers to ethylenediaminetetraacetic acid, “Fmoc” refers to fluorenylmethyloxycarbonyl chloride, “hr” refers to hour(s), “IPA” refers to isopropanol, “IPAc” refers to isopropyl acetate, “min” refers to minute(s), “Me” refers to methyl, “MTBE” refers to methyl-tert-butyl ether, “oxyma” refers to ethyl cyanohydroxyiminoacetate, “PG” refers to protecting group, “Pip” refers to piperidine, “SPPS” refers to solid phase peptide synthesis, “TFA” refers to trifluoroacetic acid, “TIPS” refers to triisopropylsilane, and “Trt” refers to trityl.

As used herein, “about” means within a statistically meaningful range of a value or values such as, for example, a stated concentration, length, molecular weight, pH, sequence identity, time frame, temperature or volume. Such a value or range can be within an order of magnitude typically within 20%, more typically within 10%, and even more typically within 5% of a given value or range. The allowable variation encompassed by “about” will depend upon the particular system under study, and can be readily appreciated by one of skill in the art.

As used herein, and with reference to one or more of the GIP, GLP-1 or GCG receptors, “activity,” “activate,” “activating” and the like means a capacity of a compound, such as an incretin analog described herein, to bind to and induce a response at the receptor(s), as measured using assays known in the art, such as the in vitro assays described below.

As used herein, “amino acid with a functional group available for conjugation” means any natural or unnatural amino acid with a functional group that may be conjugated to a fatty acid by way of, for example, a linker. Examples of such functional groups include, but are not limited to, alkynyl, alkenyl, amino, azido, bromo, carboxyl, chloro, iodo and thiol groups. Examples of natural amino acids including such functional groups include Lys/K (amino), Cys/C (thiol), Glu/E (carboxyl) and Asp/D (carboxyl).

As used herein, “analog” means a compound, such as a synthetic peptide or polypeptide, that activates a target receptor and that elicits at least one in vivo or in vitro effect elicited by a native agonist for that receptor.

As used herein, “C₁₆-C₂₂ fatty acid” means a carboxylic acid having between 16 and 22 carbon atoms. The C₁₆-C₂₂ fatty acid suitable for use herein can be a saturated monoacid or a saturated diacid. As used herein, “saturated” means the fatty acid contains no carbon-carbon double or triple bonds.

As used herein, “dual receptor activity” means an incretin analog with agonist activity at one or more of the GIP, GLP-1 and GCG receptors, especially an analog having a balanced and sufficient activity at one or more receptor to provide the benefits of agonism of that receptor while avoiding unwanted side effects associated with too much activity. Moreover, the incretin analog having dual receptor activity has extended duration of action at one or more of the GIP, GLP-1 and GCG receptors, which advantageously allows for dosing as infrequently as once-a-day, thrice-weekly, twice-weekly or once-a-week.

As used herein, “glucose-dependent insulinotropic polypeptide” or “GIP” means a peptide that plays a physiological role in glucose homeostasis by stimulating insulin secretion from pancreatic beta cells in the presence of glucose, especially human GIP (SEQ ID NO:1).

As used herein, “glucagon-like peptide-1” or “GLP-1” means a peptide that stimulates glucose-dependent insulin secretion and has been shown to prevent hyperglycemia in diabetics, especially human GLP-1 (SEQ ID NO:2).

As used herein, “glucagon” or “GCG” means peptide that helps maintain blood glucose by binding to and activating glucagon receptors on hepatocytes, causing the liver to release glucose—stored in the form of glycogen—through a process called glycogenolysis, especially human GCG (SEQ ID NO:3).

As used herein, “incretin analog” means a compound having structural similarities with, but multiple differences from, each of GIP, GLP-1 and GCG, especially human GIP (SEQ ID NO:1), human GLP-1 (SEQ ID NO:2) and human GCG (SEQ ID NO:3). The incretin analogs described herein include amino acid sequences resulting in compounds having affinity for and activity at one or more of the GIP, GLP-1 and GCG receptors (i.e., dual agonist activity or triple agonist activity).

As used herein, “pharmaceutically acceptable buffer” means any of the standard pharmaceutical buffers known to one of skill in the art.

As used herein, “triple receptor activity” means an incretin analog with agonist activity the GIP, GLP-1 and GCG receptors, especially an analog having a balanced and sufficient activity at the receptors to provide the benefits of agonism of the receptors while avoiding unwanted side effects associated with too much activity. Moreover, the incretin analog having triple receptor activity has extended duration of action at one or more of the GIP, GLP-1 and GCG receptors, which advantageously allows for dosing as infrequently as once-a-day, thrice-weekly, twice-weekly or once-a-week.

### Compositions

The structural features of the incretin analogs herein result in a compound having sufficient activity at one or more of the GIP, GLP-1 and GCG receptors to obtain the favorable effects of activity at one or more receptor (i.e., dual receptor activity or triple receptor activity), but not so much activity at any one receptor to either overwhelm the activity at the other two receptors or result in undesirable side effects when administered at a dose sufficient to result in activity at all three receptors.

The structural features of the incretin analogs herein also result in a compound having many other beneficial attributes relevant to developability as therapeutic treatments, including improving solubility of the analogs in aqueous solutions, improving chemical and physical formulation stability, extending the pharmacokinetic profile, and minimizing potential for immunogenicity.

It should be noted that the foregoing lists of structural features are exemplary, and not comprehensive, and that the combination of beneficial characteristics of exemplary analogs described herein is not the result of any modification in isolation, but is instead achieved through the novel combinations of the structural features described herein. In addition, the above-described effects of the foregoing lists of modifications are not exclusive, as many of these modifications also have other effects important to the characteristics of the compounds described herein, as described below.

The amino acid sequence of the incretin analogs herein incorporates naturally occurring amino acids, typically depicted herein using standard one or three letter codes (e.g., L/Leu=leucine), as well as α-methyl substituted residues of natural amino acids (e.g., (αMeL, αMeK, αMeY, αMeF(2F)), and certain other unnatural amino acids, such as Aib, Ornithine, 4-Pal. The structures of these amino acids are depicted below:

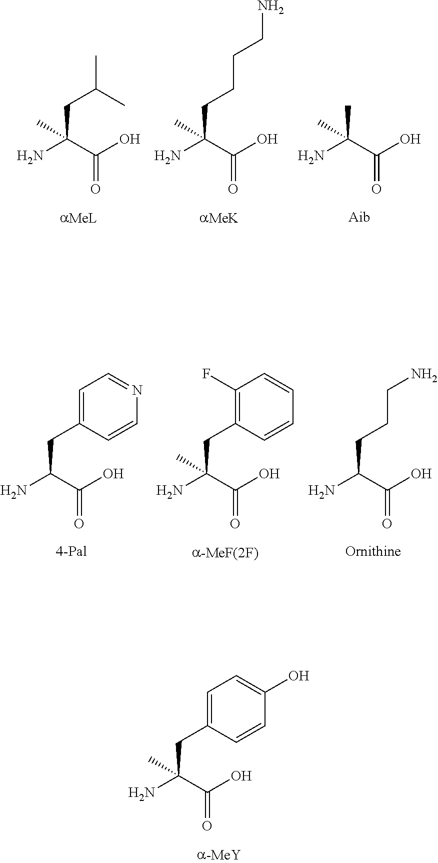

As noted above, the incretin analogs herein include a fatty acid moiety conjugated, for example, by way of a linker, to a natural or unnatural amino acid with a functional group available for conjugation. Such a conjugation is sometimes referred to as acylation. In certain instances, the amino acid with a functional group available for conjugation can be K, C, E and D, especially K at position 17 in SEQ ID NO:5 or SEQ ID NO:29, where the conjugation is to an ε-amino group of a K side-chain. The fatty acid moiety acts as an albumin binder and provides a potential to generate long-acting compounds.

The incretin analogs herein utilize a C₁₆-C₂₂ fatty acid chemically conjugated to the functional group of an amino acid either by a direct bond or by a linker. The length and composition of the fatty acid impacts half-life of the incretin analog, its potency in in vivo animal models, and their solubility and stability. Conjugation to a C₁₆-C₂₂ saturated fatty monoacid or diacid results in an incretin analog that exhibits desirable half-life, desirable potency in in vivo animal models, and desirable solubility and stability characteristics.

Examples of saturated C₁₆-C₂₂ fatty acids for use herein include, but are not limited to, palmitic acid (hexadecanoic acid) (C₁₆ monoacid), hexadecanedioic acid (C₁₆ diacid), margaric acid (heptadecanoic acid) (C₁₇ monoacid), heptadecanedioic acid (C₁₇ diacid), stearic acid (C₁₈ monoacid), octadecanedioic acid (C₁₈ diacid), nonadecylic acid (nonadecanoic acid) (C₁₉ monoacid), nonadecanedioic acid (C₁₉ diacid), arachadic acid (eicosanoic acid) (C₂₀ monoacid), eicosanedioic acid (C₂₀ diacid), heneicosylic acid (heneicosanoic acid) (C₂₁ monoacid), heneicosanedioic acid (C₂₁ diacid), behenic acid (docosanoic acid) (C₂₂ monoacid), docosanedioic acid (C₂₂ diacid), including branched and substituted derivatives thereof.

In some instances, the C₁₆-C₂₂ fatty acid can be a saturated Cis monoacid, a saturated C₁₈ diacid, a saturated C₁₉ monoacid, a saturated C₁₉ diacid, a saturated C₂₀ monoacid, a saturated C₂₀ diacid, and branched and substituted derivatives thereof.

In some instances, the linker can have from one to four amino acids, an amino polyethylene glycol carboxylate, or mixtures thereof. In certain instances, the amino polyethylene glycol carboxylate has the following structure:

H—{NH—CH₂—CH₂—[O—CH₂—CH₂]ₘ—O—(CH₂)ₚ—CO}ₙ—OH,

where m is any integer from 1 to 12, n is any integer from 1 to 12, and p is 1 or 2.

In some instances, the linker can have one or more (2-[2-(2-amino-ethoxy)-ethoxy]-acetyl) moieties, optionally in combination with one to four amino acids.

In instances in which the linker includes at least one amino acid, the amino acid can be one to four Glu or γGlu amino acid residues. In some instances, the linker can include one or two Glu or γGlu amino acid residues, including the D-forms thereof. For example, the linker can include either one or two γGlu amino acid residues. Alternatively, the linker can include one to four amino acid residues (such as, for example, Glu or γGlu amino acids) used in combination with up to thirty-six (2-[2-(2-amino-ethoxy)-ethoxy]-acetyl) moieties. Specifically, the linker can be combinations of one to four Glu or γGlu amino acids and one to four (2-[2-(2-amino-ethoxy)-ethoxy]-acetyl) moieties. In other instances, the linker can be combinations of one or two γGlu amino acids and one or two (2-[2-(2-amino-ethoxy)-ethoxy]-acetyl) moieties.

In certain instances, the incretin analog described herein includes linker and fatty acid components having the structure of the following formula:

(2-[2-(2-amino-ethoxy)-ethoxy]-acetyl)ₐ-(γGlu)_{b}-CO—(CH₂)_{c}—CO₂H,

where a is 0, 1 or 2, b is 1 or 2, and c is 16 or 18.

In a particular instance, a is 2, b is 1, and c is 16, the structure of which is depicted below:

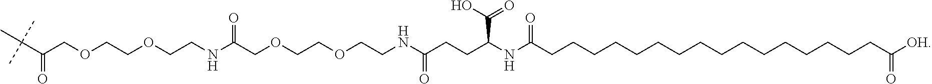

In another particular instance, a is 1, b is 2, and c is 18, the structure of which is depicted below:

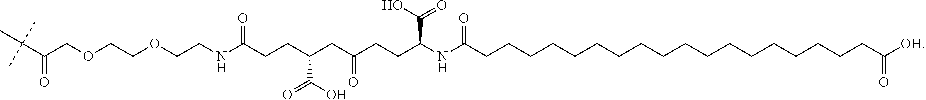

In another particular instance, a is 0, b is 2, and c is 18, the structure of which is depicted below:

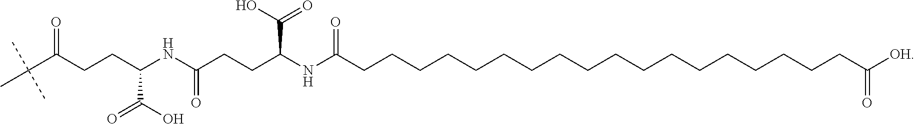

In another particular instance, a is 1, b is 1, and c is 18, the structure of which is depicted below:

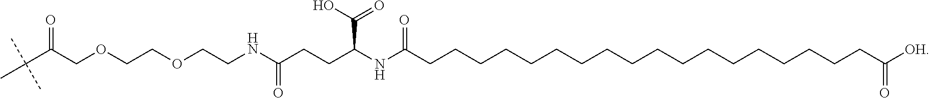

In particular instances, the overall structure of the incretin analog is SEQ ID NO:6.

In particular instances, the overall structure of the incretin analog is SEQ ID NO:29.

The affinity of the incretin analogs herein for each of the GIP, GLP-1 and GCG receptors may be measured using techniques known in the art for measuring receptor binding levels, including, for example, those described in the examples below, and is commonly expressed as an inhibitory constant (Ki) value. The activity of the incretin analogs herein at one or more of the receptors also may be measured using techniques known in the art, including, for example, the in vitro activity assays described below, and is commonly expressed as an effective concentration 50 (EC₅₀) value, which is the concentration of compound causing half-maximal simulation in a dose response curve.

The incretin analogs herein can be formulated as a pharmaceutical composition, which can be administered by parenteral routes (e.g., subcutaneous, intravenous, intraperitoneal, intramuscular or transdermal). Such pharmaceutical composition and methods of preparing the same are well known in the art. See, e.g., “Remington: The Science and Practice of Pharmacy” (Troy ed., Lippincott, Williams & Wilkins 21^{st} ed. 2006).

The incretin analogs herein may react with any of a number of inorganic and organic acids/bases to form pharmaceutically acceptable acid/base addition salts. Pharmaceutically acceptable salts and common techniques for preparing them are well known in the art (see, e.g., Stahl et al., “Handbook of Pharmaceutical Salts: Properties, Selection and Use” (Wiley-VCH 2^{nd} ed. 2011)). Pharmaceutically acceptable salts for use herein include sodium, trifluoroacetate, hydrochloride and/or acetate salts.

The disclosure also provides and therefore encompasses novel intermediate compounds and methods of synthesizing the incretin analogs herein or pharmaceutically acceptable salts thereof. The intermediate compounds and incretin analogs herein can be prepared by a variety of techniques known in the art. For example, a method using standard solid phase peptide synthesis for two or more intermediate compounds followed by HLSPS thereof is illustrated in the Examples below. The specific synthetic steps for each of the routes described may be combined in different ways to prepare the incretin analogs herein. The reagents and starting materials are readily available to one of skill in the art.

The incretin analogs herein are generally effective over a wide dosage range. For example, dosages for once-weekly administration may fall within a range of about 0.01 to about 30 mg/person/week, within a range of about 0.1 to about 10 mg/person/week or even within a range of about 0.1 to about 3 mg/person/week. Thus, the incretin analogs described herein may be dosed daily, thrice-weekly, twice-weekly or once-weekly, especially once-weekly administration.

The incretin analogs herein may be used for treating a variety of conditions, disorders, diseases or symptoms. In particular, methods are provided below for treating T2DM in an individual, where such methods include at least a step of administering to an individual in need of such treatment an effective amount of an incretin analog herein, or a pharmaceutically acceptable salt thereof.

### Methods

### Standard Solid Phase Peptide Synthesis of Intermediate Compounds:

The incretin analogs herein can be made via any number of standard peptide synthesis methods known in the art, especially SPPS. SPPS builds are accomplished using standard Fmoc peptide chemistry techniques employing sequential couplings with an automated peptide synthesizer. Methods of SPPS are well known in the art and need not be exhaustively described herein. See generally, “Fmoc Solid Phase Peptide Synthesis: A Practical Approach” (Chan & White ed., Oxford University Press 2000), and Merrifield (1963) J. Am. Chem. Soc. 85:2149-2154.

For deprotection, a resin is swelled with DMF, and then deprotected using 20% Pip/DMF (3×30 min). Subsequent Fmoc deprotections use 20% Pip/DMF (1×5-20 min, 1×20-30 min) treatments, with 1×5-20 min, 1×20 min and 1×30 min treatment sequences being used for more difficult deprotections.

After deprotection, the resin is washed with 5×2 min, 10 volume DMF washes. Amino acid pre-activation uses DIC/Oxyma DMF solutions at room temperature for 30 min. Coupling of the activated amino acid to the resin-bound peptide occurs for a specified time for each individual amino acid. Solvent washing with 5×2 min with 10 volumes DMF is performed after each coupling.

For isolation of the final product, the resin-bound product is washed 5×2 min with 10 volume DCM to remove DMF. The resin is washed with 2×2 min 10 volume IPA to remove DCM, washed 5×2 min with 10 volume MTBE, and then the product is dried at 40° C. under vacuum. The resin-bound product is stored cold (−20° C.).

For analysis, peptide is cleaved from the resin with an acidic cocktail of TFA/H₂O/TIPS/DTT in the following ratio: (0.93 v/0.04 v/0.03 v/0.03 w). The resin is swelled with DCM (4-5 vol, 3×30 min) and drained. Cleavage cocktail (4-5 vol) is added to the pre-swelled resin, and the suspension is stirred for 2 hr at room temperature. The solution is filtered, and then the resin is washed with a small amount of DCM and combined with the cleavage solution. The resulting solution is poured into 7-10 volumes of cold (0° C.) MTBE. The suspension is aged for 30 min at 0° C., the resulting precipitate is centrifuged, and the clear solution is decanted. The residue is suspended in the same volume of MTBE, and the resulting suspension is again centrifuged and decanted. After decanting, the clear MTBE solution of the precipitated peptide is dried in vacuo at 40° C. overnight.

### Hybrid Liquid Solid Phase Synthesis of the Incretin Analogs:

Intermediate compounds prepared via SPPS as described above can be combined to obtain the incretin analog of SEQ ID NO:6 or 29. Methods of HLSPS are well known in the art and need not be exhaustively described herein. See generally, US Patent Application Publication No. 2011/0046349; and Albericio et al. (1997) Methods Enzymol. 289:313-336, Bray et al. (2003) Nature Rev. Drug Discovery 2:587-593, Dalcol et al. (1995) J. Org. Chem. 7575-60:7581, Gauthier et al. (1991) Tettrahedron Lett. 32: 577-580, Schneider et al. (2005) J. Peptide Sci. 11:744-753, Smith, Organic Synthesis (Academic Press 4^{th} ed. 2016), and Zhang et al. (2008) Org. Process Res. Dev. 12:101-110.

Briefly, HLSPS involves independent intermediate compound synthesis and compound coupling. Applied here, one method of making the incretin analog of SEQ ID NO:6 includes at least a step of coupling the following four intermediate compounds, where such compounds have a structure as recited in SEQ ID NOS:7, 8, 9 and 10.

In some instances, the fragments can be coupled in the following order: SEQ ID NO:7 to SEQ ID NO:8 to SEQ ID NO:9 to SEQ ID NO:10 (i.e., from C-terminus to N-terminus). In other instances, and with an appropriate protecting group strategy, the fragments can be coupled in a different order.

Another method of making the incretin analog of SEQ ID NO:6 includes at least a step of coupling the following four intermediate compounds, where such compounds have a structure as recited in SEQ ID NOS:7, 11, 12 and 10.

In some instances, the fragments are coupled in the following order: SEQ ID NO:7 to SEQ ID NO:11 to SEQ ID NO:12 to SEQ ID NO:10 (i.e., from C-terminus to N-terminus). In other instances, and with an appropriate protecting group strategy, the fragments can be coupled in a different order.

Another method of making the incretin analog of SEQ ID NO:6 includes at least a step of coupling the following four intermediate compounds, where such compounds have a structure as recited in SEQ ID NOS:7, 13, 14 and 10.

In some instances, the fragments are coupled in the following order: SEQ ID NO:7 to SEQ ID NO:13 to SEQ ID NO:14 to SEQ ID NO:10 (i.e., from C-terminus to N-terminus). In other instances, and with an appropriate protecting group strategy, the fragments can be coupled in a different order.

Alternatively, one method of making the incretin analog of SEQ ID NO:6 includes at least a step of coupling the following three intermediate compounds, where such compounds have a structure as recited in SEQ ID NOS:7 13 and 15.

In some instances, the fragments are coupled in the following order: SEQ ID NO:7 to SEQ ID NO:13 to SEQ ID NO:15 (i.e., from C-terminus to N-terminus). In other instances, and with an appropriate protecting group strategy, the fragments can be coupled in a different order.

Another method of making the incretin analog of SEQ ID NO:6 includes at least a step of coupling the following three intermediate compounds, where such compounds have a structure as recited in SEQ ID NOS:16, 17 and 10.

In some instances, the fragments are coupled in the following order: SEQ ID NO:16 to SEQ ID NO:17 to SEQ ID NO:10 (i.e., from C-terminus to N-terminus). In other instances, and with an appropriate protecting group strategy, the fragments can be coupled in a different order.

Another method of making the incretin analog of SEQ ID NO:6 includes at least a step of coupling the following three intermediate compounds, where such compounds have a structure as recited in SEQ ID NOS:18, 12 and 10.

In some instances, the fragments are coupled in the following order: SEQ ID NO:18 to SEQ ID NO:12 to SEQ ID NO:10 (i.e., from C-terminus to N-terminus). In other instances, and with an appropriate protecting group strategy, the fragments can be coupled in a different order.

Another method of making the incretin analog of SEQ ID NO:6 includes at least a step of coupling the following three intermediate compounds, where such compounds have a structure as recited in SEQ ID NOS:7, 45 and 10.

In some instances, the fragments are coupled in the following order: SEQ ID NO:7 to SEQ ID NO:45 to SEQ ID NO:10 (i.e., from C-terminus to N-terminus). In other instances, and with an appropriate protecting group strategy, the fragments can be coupled in a different order.

Another method of making the incretin analog of SEQ ID NO:6 includes at least a step of coupling the following three intermediate compounds, where such compounds have a structure as recited in SEQ ID NOS:7, 11 and 20.

In some instances, the fragments are coupled in the following order: SEQ ID NO:7 to SEQ ID NO:11 to SEQ ID NO:20 (i.e., from C-terminus to N-terminus). In other instances, and with an appropriate protecting group strategy, the fragments can be coupled in a different order.

Alternatively, one method of making the incretin analog of SEQ ID NO:6 includes at least a step of coupling the following two intermediate compounds, where such compounds have a structure as recited in SEQ ID NOS:19 and 15.

Another method of making the incretin analog of SEQ ID NO:6 includes at least a step of coupling the following two intermediate compounds, where such compounds have a structure as recited in SEQ ID NOS:18 and 20.

Alternatively, other methods of making the incretin analog of SEQ ID NO:6 use the same disconnections as described above but instead couple all amino acid fragments of the backbone first, and then introduce the fatty acid side moiety as the last chemical transformation followed by global deprotection. Here, for example, the corresponding PG can be implemented at Lys17, which can be selectively removed in presence of other PGs (e.g., Boc, tBu and/or Trt). In some instances, a method of making the incretin analog of SEQ ID NO:6 includes at least a step of coupling the following intermediate compounds, where such compounds have a structure as recited in SEQ ID NOS:21 and 18, as well as

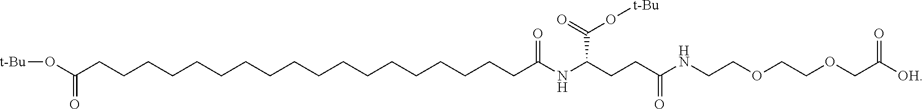 [SMILES: CC(C)(C)OC(=O)CCCCCCCCCCCCCCCCCCC(=O)N[C@@H](CCC(=O)NCCOCCOCC(=O)O)C(=O)OC(C)(C)C]

In some instances, a method of making the incretin analog of SEQ ID NO:6 includes at least a step of coupling the following intermediate compounds, where such compounds have as structure as recited in SEQ ID NOS:22 and 19, as well as

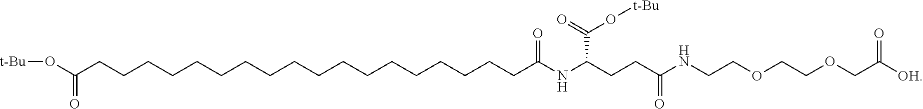 [SMILES: CC(C)(C)OC(=O)CCCCCCCCCCCCCCCCCCC(=O)N[C@@H](CCC(=O)NCCOCCOCC(=O)O)C(=O)OC(C)(C)C]

Alternatively, other methods of making the incretin analog of SEQ ID NO:6 include at least of step of coupling deprotected compound intermediates (e.g., a thioester fragment and amide fragment) via a NCL approach. Here, for example Ala21 can be substituted with a natural enantiomer of Cys, and after completing the ligation step SEQ ID NO:6 can be obtained by desulfurization of the Cys to deliver required Ala21 with the following intermediate compounds, where such compounds have a structure as recited in SEQ ID NOS:23 and 24.

Alternatively, the thioester (SEQ ID NO:23) may be substituted by an intermediate compound, in which the moiety of —C—P—OR Ester (CPE) at the C-terminal can serve as a masked thioester to facilitate the ligation step of compounds having a structure as recited in SEQ ID NOS:39 and 24.

In other instances, Ala18 can be replaced with Cys and desulfurized after the native chemical ligation of the following intermediate compounds, where such compounds have a structure as recited in SEQ ID NOS:25 and 26.

Alternatively, the thioester (SEQ ID NO:25) can be substituted by an intermediate compound, in which a moiety of -Cys-Pro-OR Ester (CPE) can serve as a masked thioester to facilitate the ligation step of compounds having a structure as recited in SEQ ID NOS:40 and 26.

Alternatively, other methods of making the incretin analog of SEQ ID NO:5 include at least of step of coupling the following intermediate compounds (e.g., a non-acylated thioester fragment and amide fragment) via a NCL approach, where such compounds have a structure as recited in SEQ ID NOS:27 and 26.

Alternatively, the thioester (SEQ ID NO:27) can be substituted by an intermediate compound, in which the moiety of -Cys-Pro-OR Ester (CPE) can serve as a masked thioester to facilitate the ligation step of compounds having a structure as recited in SEQ ID NOS:41 and 26.

In another embodiment, SEQ ID NO:29 can be synthesized by coupling SEQ ID NO:43 and SEQ ID NO:44 and then deprotect to produce SEQ ID NO:29.

In another embodiment, SEQ ID NO:48 can be synthesized by using SEQ ID NO:20 and SEQ ID NO:18. SEQ ID NO:48 is deprotected to produce SEQ ID NO:6.

In another embodiment, SEQ ID NO:53 can be synthesized by NCL using SEQ ID NO:51 and SEQ ID NO:52.

In another embodiment, SEQ ID NO:53 can be synthesized by NCL using SEQ ID NO:52 and SEQ ID NO:54.

For effective preparation of compound intermediates of SEQ ID NOS:9, 12, 14, 15, 17, 20, 23 and 25, the following is synthesized using fatty side chain

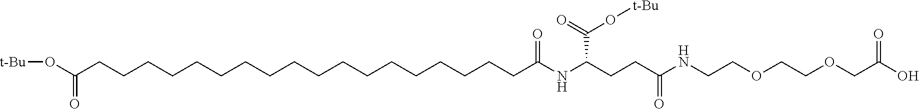 [SMILES: CC(C)(C)OC(=O)CCCCCCCCCCCCCCCCCCC(=O)N[C@@H](CCC(=O)NCCOCCOCC(=O)O)C(=O)OC(C)(C)C]

and Fmoc-L-Lys-OH amino acid attached with fatty side chain:

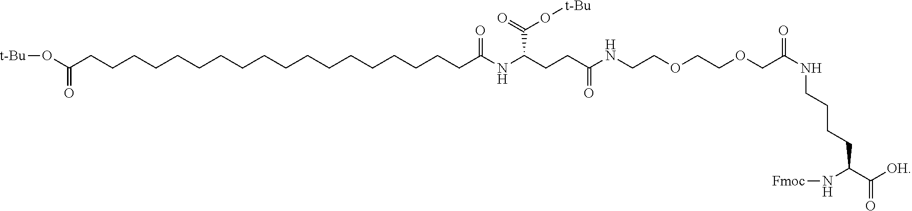

For improved purity and efficiency of the SPPS, the following dimer, trimer and tetramer can be used for preparing SEQ ID NOS:10, 15, 20, 21, 22, 23, 25 and 27, where the structures that follow can be synthesized using amino acid building block via SPPS or liquid phase synthesis:

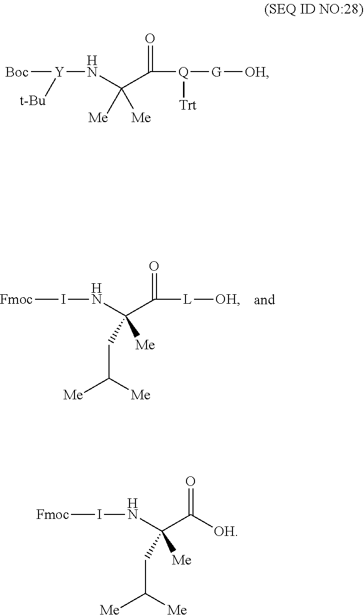

### Other Methods/Uses:

The incretin analogs herein can be used in a number of therapeutic applications. For example, the incretin analogs can be used in methods of treating obesity in an individual, where such methods include at least a step of administering to an individual in need of such treatment an effective amount of an incretin analog herein, or a pharmaceutically acceptable salt thereof.

Additionally, the incretin analogs can be used in methods of inducing non-therapeutic weight loss in an individual, where such methods include at least a step of administering to an individual in need of such treatment an effective amount of an incretin analog herein, or a pharmaceutically acceptable salt thereof.

Additionally, the incretin analogs herein can be used in methods of treating metabolic syndrome in an individual, where such methods include at least a step of administering to an individual in need of such treatment an effective amount of an incretin analog herein, or a pharmaceutically acceptable salt thereof.

Additionally, the incretin analogs herein can be used in methods of treating NASH in an individual, where such methods include at least a step of administering to an individual in need of such treatment an effective amount of an incretin analog described, or a pharmaceutically acceptable salt thereof.

Additionally, the incretin analogs herein can be used in methods of treating NAFLD in an individual, where such methods include at least a step of administering to an individual in need of such treatment an effective amount of an incretin analog herein, or a pharmaceutically acceptable salt thereof.

In these methods, effectiveness of the incretin analogs can be assessed by, for example, observing a significant reduction in blood glucose, observing a significant increase in insulin, observing a significant reduction in HbA1c and/or observing a significant reduction in body weight.

Alternatively, the incretin analogs herein or pharmaceutically acceptable salts thereof may be used for improving bone strength in an individual in need thereof. In some instances, the individual in need thereof has hypo-ostosis or hypo-osteoidosis, or is healing from bone fracture, orthotic procedure, prosthetics implant, dental implant, and/or spinal fusion. The incretin analogs also may be used for treating other disorders such as Parkinson's disease or Alzheimer's disease.

### EXAMPLES

The following non-limiting examples are offered for purposes of illustration, not limitation.

### Peptide and Polypeptide Synthesis

### Example 1: Solid Phase Peptide Synthesis of Intermediate Compound 1

Intermediate Compound 1 (SEQ ID NO:7), or a pharmaceutically acceptable salt thereof, can be synthesized by standard SPPS. Briefly, SPPS is conducted using Sieber resin (loading factor 0.6-0.9 mmol/g) with the conditions set forth below in Table 1.

TABLE 1
SPPS Conditions for Example 1.

| | | | SPPS Conditions |
|---|---|---|---|
| Cycle | Deprotection | Amino Acid | Solvent for Couplings: DMF |
| 1 | 2 × 30 min | Fmoc-L-Ser(t-Bu)-OH | 2.0 AA/2.2 DIC/2.0 Oxyma |
| | 20% Pip/DMF | | 4 hr, rt |
| 2 | 2 × 30 min | Fmoc-L-Pro-OH | 2.0 AA/2.2 DIC/2.0 Oxyma |
| | 20% Pip/DMF | | 6 hr, rt |
| 3 | 2 × 30 min | Fmoc-L-Pro-OH | 2.0 AA/2.2 DIC/2.0 Oxyma |
| | 0% Pip/DMF | | 6 hr, rt |
| 4 | 2 × 30 min | Fmoc-L-Pro-OH | 2.0 AA/2.2 DIC/2.0 Oxyma |
| | 20% Pip/DMF | | 6 hr, rt |
| 3-4* | Alternatively use Fmoc-L-Pro-Pro-OH dimer instead of step 3 & 4 | | |
| 5 | 2 × 30 min | Fmoc-L-Ala-OH | 2.0 AA/2.2 DIC/2.0 Oxyma |
| | 20% Pip/DMF | | 4 hr, rt |
| 6 | 2 × 30 min | Fmoc-Gly-OH | 2.0 AA/2.2 DIC/2.0 Oxyma |
| | 20% Pip/DMF | | 4 hr, rt |
| 7 | 2 × 30 min | Fmoc-L-Ser(t-Bu)-OH | 2.0 AA/2.2 DIC/2.0 Oxyma |
| | 20% Pip/DMF | | 4 hr, rt |
| 8 | 2 × 30 min | Fmoc-L-Ser(t-Bu)-OH | 2.0 AA/2.2 DIC/2.0 Oxyma |
| | 20% Pip/DMF | | 4 hr, rt |
| 9 | 2 × 30 min | Fmoc-L-Pro-OH | 2.0 AA/2.2 DIC/2.0 Oxyma |
| | 20% Pip/DMF | | 4 hr, rt |
| 10 | 2 × 30 min | Fmoc-Gly-OH | 2.0 AA/2.2 DIC/2.0 Oxyma |
| | 20% Pip/DMF | | 4 hr, rt |

Fmoc Deprotection, Fragment Cleavage and Isolation: Fragment on Sieber resin is stirred twice with 10 V of 20% piperidine/DMF for 20-30 min, then washed six times with 10 V of DMF. The de-Fmoced fragment on Sieber resin is swelled twice using 10 V DCM for 10-20 min. A reactor with resin is cooled to about 15° C., and 20 V of 5% TFA/DCM is charged to the reactor and then stirred for 2 hr under nitrogen maintaining the temperature at about 15° C. The resin is filtered and washed with 3×10 V of DCM. All the filtrates are combined together. DCM is removed from the resulting solution under reduced pressure while maintaining the internal temperature at ≤20° C. to 22.5 V residual volume. MTBE (25 V) is charged to the solution, and DCM/MTBE solvents are again removed under reduced pressure while maintaining the internal temperature at ≤20° C. to 22.5 V residual volume. Addition of MTBE/distillation operation is repeated until residual concentration of fragment in supernatant has not reached <0.11 wt %. Then, the resulting slurry is filtered while maintaining temperature at about 15° C. To the cake, 14 V of fresh MTBE is added, is stirred for 30 min at about 15° C., and then is filtered. Washing is repeated one more time, and the resulting solid is dried at about 35° C.

### Example 2: Solid Phase Peptide Synthesis of Intermediate Compound 2

Intermediate Compound 2 (SEQ ID NO:8), or a pharmaceutically acceptable salt thereof, can be synthesized by standard SPPS. Briefly, SPPS is conducted using Fmoc-Gly-2-CTC resin (loading factor 0.6-0.9 mmol/g) with the conditions set forth below in Table 2.

TABLE 2
SPPS Conditions for Example 2.

| | | | SPPS Conditions |
|---|---|---|---|
| Cycle | Deprotection | Amino Acid | Solvent for Couplings: DMF |
| 1 | 2 × 20-30 min | Fmoc-L-Glu(t-Bu)-OH | 2.5 AA/2.5 PyBOP/5.0 DIPEA |
| | 20% Pip/DMF | | 4 hr, rt |
| 2 | 2 × 20-30 min | Fmoc-L-Leu-OH | 2.0 AA/2.2 DIC/2.0 Oxyma |
| | 20% Pip/DMF | | 4-8 hr, rt |
| 3 | 2 × 20-30 min | Fmoc-L-Leu-OH | 2.0 AA/2.2 DIC/2.0 Oxyma |
| | 20% Pip/DMF | | 4-8 hr, rt |
| 4 | 2 × 20-30 min | Fmoc-L-Tyr(t-Bu)-OH | 2.0 AA/2.2 DIC/2.0 Oxyma |
| | 20% Pip/DMF | | 4-8 hr, rt |
| 5 | 2 × 20-30 min | Fmoc-L-Glu(t-Bu)-OH | 2.0 AA/2.2 DIC/2.0 Oxyma |
| | 20% Pip/DMF | | 4-8 hr, rt |
| 6 | 2 × 20-30 min | Fmoc-L-Ile-OH | 2.0 AA/2.2 DIC/2.0 Oxyma |
| | 20% Pip/DMF | | 4-8 hr, rt |
| 7 | 2 × 20-30 min | Fmoc-L-Phe-OH | 2.0 AA/2.2 DIC/2.0 Oxyma |
| | 20% Pip/DMF | | 4-8 hr, rt |

Fragment Cleavage and Isolation: Fragment on CTC resin is swelled once using DCM (5 V) for 45 min. 10 V of 1% TFA/DCM is charged to the reactor, and the resulting suspension of the resin is stirred for 10-15 min under nitrogen at about 25° C. The filtrate is removed and immediately neutralized by slow addition of a 1.05 equivalent amount of pyridine, and then 5 V of DMSO is added to the filtrate. Resin treatment with 1% TFA/DCM followed by filtrate neutralization is repeated two more times. The resin is washed with 3 V of DCM, and stirred for 10-15 min. All the filtrates and wash are combined. The fragment solution is concentrated under vacuum to 6-10 V maintaining temperature at ≤35° C. (residual DCM concentration ≤15%). DMSO solution of the fragment is added to 11-15 V of H₂O over 2-6 hr period (<1 L/min) at about 25° C. The formed slurry of precipitated fragment is stirred for 30-40 min at about 25° C. and then filtered. The resulting solid is suspended in 8-12 V of H₂O at about 25° C., is stirred 10-15 min, and then is filtered. Washing is repeated one more time, and the resulting solid is dried at about 40° C.

### Example 3: Solid Phase Peptide Synthesis of Intermediate Compound 3

Intermediate Compound 3 (SEQ ID NO:9), or a pharmaceutically acceptable salt thereof, can be synthesized by standard SPPS. Briefly, SPPS is conducted using Fmoc-Ala-2-CTC resin (loading factor 0.6-0.9 mmol/g) with the conditions set forth below in Table 3.

TABLE 3
SPPS Conditions for Example 3.

| | | | SPPS Conditions |
|---|---|---|---|
| Cycle | Deprotection | Amino Acid | Solvent for Couplings: DMF |
| 1 | 2 × 20-30 min | Fmoc-Aib-OH | 2.5 AA/2.5 PyBOP/5.0 DIPEA |
| | 20% Pip/DMF | | 4 hr, rt |
| 2 | 2 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |
| 3 | 2 × 20-30 min | Fmoc-L-Ala-OH | 2.0 AA/2.2 DIC/2.0 Oxyma |
| | 20% Pip/DMF | | 4 hr, rt |
| 4 | 2 × 20-30 min | Fmoc-L-Lys(t- | 1.5 AA/1.65 DIC/1.5 Oxyma |
| | 20% Pip/DMF | BuOOC—(CH₂)₁₈—COO- | 4 hr, rt |
| | | γ-L-Glu-AEEA)* | |
| 5 | 2 × 20-30 min | Fmoc-L-Lys(Boc)-OH | 2.0 AA/2.2 DIC/2.0 Oxyma |
| | 20% Pip/DMF | | 4 hr, rt |
| 6 | 2 × 20-30 min | Fmoc-L-Asp(t-Bu)-OH | 2.0 AA/2.2 DIC/2.0 Oxyma |
| | 20% Pip/DMF | | 4 hr, rt |

*Fmoc-L-Lys(t-BuOOC—(CH₂)₁₈—COO-γ-L-Glu-AEEA)

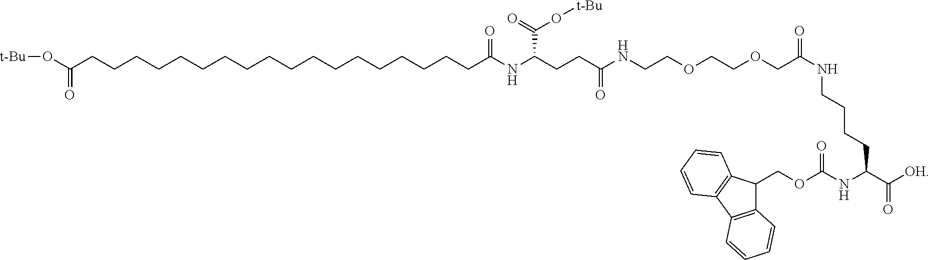 [SMILES: CC(C)(C)OC(=O)CCCCCCCCCCCCCCCCCCC(=O)N[C@@H](CCC(=O)NCCOCCOCC(=O)NCCCC[C@H](NC(=O)OCC1c2ccccc2-c2ccccc21)C(=O)O)C(=O)OC(C)(C)C]

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% TFA/DCM followed by filtrate neutralization is repeated two more times. The resin is washed with 3 V of DCM and stirred for 10-15 min. All the filtrates and wash are combined, and the resulting mixture cooled ≤20° C. The fragment solution is concentrated under vacuum to 2-4 V maintaining temperature at ≤20° C. 5 V of ACN is added to the solution, and residual DCM removed under vacuum (residual DCM concentration ≤X %) maintaining temperature at ≤20° C. An ACN solution of the fragment is added to 5 V of ice-cold H₂O over 2-6 hr period (<1 L/min) maintaining temperature at about 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at about 40° C.

### Example 4: Solid Phase Peptide Synthesis of Intermediate Compound 4

Intermediate Compound 4 (SEQ ID NO:10), or a pharmaceutically acceptable salt thereof, can be synthesized by standard SPPS. Briefly, SPPS is conducted using Fmoc-Leu-2-CTC resin (loading factor 0.6-0.9 mmol/g) with the conditions set forth below in Table 4.

TABLE 4
SPPS Conditions for Example 4.

| | | | SPPS Conditions |
|---|---|---|---|
| Cycle | Deprotection | Amino Acid | Solvent for Couplings:DMF |
| 1 | 2 × 20-30 min | Fmoc-L-2-Me-Ile- | 2.0 AA/2.2 DIC/2.0 Oxyma |
| | 20% Pip/DMF | OH | 12-18 hr, rt |
| | | | capping procedure |
| 2 | 3 × 20-30 min | Fmoc-L-Ile-OH | 2.0 AA/2.2 DIC/2.0 Oxyma |
| | 20% Pip/DMF | | 12-18 hr, rt |
| | | | capping procedure |
| 3 | 2 × 20-30 min | Fmoc-L-Ser(t-Bu)- | 2.0 AA/2.2 DIC/2.0 Oxyma |
| | 20% Pip/DMF | OH | 4-6 hr, rt |
| 4 | 2 × 20-30 min | Fmoc-L-Tyr(t-Bu)- | 2.0 AA/2.2 DIC/2.0 Oxyma |
| | 20% Pip/DMF | OH | 4-6 hr, rt |
| 5 | 2 × 20-30 min | Fmoc-L-Asp(t-Bu)- | 2.0 AA/2.2 DIC/2.0 Oxyma |
| | 20% Pip/DMF | OH | 4-6 hr, rt |
| 6 | 2 × 10 min | Fmoc-L-Ser(t-Bu)- | 2.0 AA/2.2 DIC/2.0 Oxyma |
| | 5% Oxyma/20% | OH | 4-6 hr, rt |
| | Pip/DMF | | |
| 7 | 2 × 10 min | Fmoc-L-Thr(t-Bu)- | 2.0 AA/2.2 DIC/2.0 Oxyma |
| | 5% Oxyma/20% | OH | 4-6 hr, rt |
| | Pip/DMF | | |
| 8 | 2 × 10 min | Fmoc-L-Phe(t-Bu)- | 2.0 AA/2.2 DIC/2.0 Oxyma |
| | 5% Oxyma/20% | OH | 4-6 hr, rt |
| | Pip/DMF | | |
| 9 | 2 × 10 min | Fmoc-L-Thr(t-Bu)- | 2.0 AA/2.2 DIC/2.2 Oxyma |
| | 5% Oxyma/20% | OH | 4-6 hr, rt |
| | Pip/DMF | | |
| 10 | 2 × 10 min | Boc-L-Tyr(t-Bu)- | 1.5 AA/1.65 DIC/1.5 Oxyma |
| | 5% Oxyma/20% | Aib-L-Gln(Trt)-Gly- | 4-6 hr, rt |
| | Pip/DMF | OH* | |

*structure for Boc-L-Tyr(t-Bu)-Aib-L-Gln(Trt)-Gly-OH is as follows:

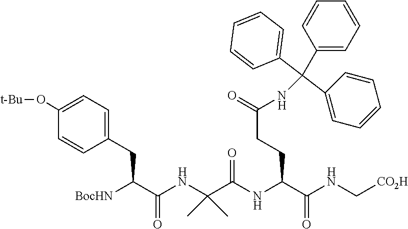 [SMILES: CC(C)(C)OC(=O)N[C@@H](Cc1ccc(OC(C)(C)C)cc1)C(=O)NC(C)(C)C(=O)N[C@@H](CCC(=O)NC(c1ccccc1)(c1ccccc1)c1ccccc1)C(=O)NCC(=O)O]

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% with TFA/DCM followed by filtrate neutralization is repeated two more times. The resin is washed with 3 V of DCM, and is stirred for 10-15 min. All the filtrates and wash are combined, and the resulting mixture is cooled to ≤20° C. The fragment solution is concentrated under vacuum to 2-4 V maintaining temperature at ≤20° C. 2 V of DMSO is added to the solution, and residual DCM removed under vacuum (residual DCM concentration ≤5%) maintaining temperature at ≤20° C. A DMSO solution of the fragment is added to 7-9 V of ice-cold H₂O over 2-6 hr (<1 L/min), while maintaining temperature at about 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at 40° C.

### Example 5: Solid Phase Peptide Synthesis of Intermediate Compound 5

Intermediate Compound 5 (SEQ ID NO:11), or a pharmaceutically acceptable salt thereof, can be synthesized by standard SPPS. Briefly, SPPS is conducted using Fmoc-Gly-2-CTC resin (loading factor 0.6-0.9 mmol/g) with the conditions set forth below in Table 5.

TABLE 5
SPPS Conditions for Example 5.

| | | | SPPS Conditions |
|---|---|---|---|
| Cycle | Deprotection | Amino Acid | Solvent for Couplings: DMF |
| 1 | 2 × 20-30 min | Fmoc-L-Glu(t-Bu)-OH | 2.5 AA/2.5 PyBOP/5.0 DIPEA |
| | 20% Pip/DMF | | 4 hr, rt |
| 2 | 2 × 20-30 min | Fmoc-L-Leu-OH | 2.0 AA/2.2 DIC/2.0 Oxyma |
| | 20% Pip/DMF | | 4-8 hr, rt |
| 3 | 2 × 20-30 min | Fmoc-L-Leu-OH | 2.0 AA/2.2 DIC/2.0 Oxyma |
| | 20% Pip/DMF | | 4-8 hr, rt |
| 4 | 2 × 20-30 min | Fmoc-L-Tyr(t-Bu)-OH | 2.0 AA/2.2 DIC/2.0 Oxyma |
| | 20% Pip/DMF | | 4-8 hr, rt |
| 5 | 2 × 20-30 min | Fmoc-L-Glu(t-Bu)-OH | 2.0 AA/2.2 DIC/2.0 Oxyma |
| | 20% Pip/DMF | | 4-8 hr, rt |
| 6 | 2 × 20-30 min | Fmoc-L-Ile-OH | 2.0 AA/2.2 DIC/2.0 Oxyma |
| | 20% Pip/DMF | | 4-8 hr, rt |
| 7 | 2 × 20-30 min | Fmoc-L-Phe-OH | 2.0 AA/2.2 DIC/2.0 Oxyma |
| | 20% Pip/DMF | | 4-8 hr, rt |
| 8 | 2 × 20-30 min | Fmoc-L-Ala-OH | 2.0 AA/2.2 DIC/2.0 Oxyma |
| | 20% Pip/DMF | | 4-8 hr, rt |

Fragment Cleavage and Isolation: Fragment on CTC resin is swelled once using DCM (5 V) for 45 min. 10 V of 1% TFA/DCM is charged to the reactor, and the resulting suspension of the resin is stirred for 10-15 min under nitrogen at about 25° C. The filtrate is removed and immediately neutralized by slow addition of a 1.05 equivalent amount of pyridine, and then 5 V of DMSO is added to the filtrate. Resin treatment with 1% TFA/DCM followed by filtrate neutralization is repeated two more times. The resin is washed with 3 V of DCM and is stirred for 10-15 min. All the filtrates and wash are combined. The fragment solution is concentrated under vacuum to 6-10 V maintaining temperature at ≤35° C. (residual DCM concentration ≤15%). DMSO solution of the fragment is added to 11-15 V of H₂O over 2-6 hr (<1 L/min) at about 25° C. The formed slurry of precipitated fragment is stirred for 30-40 min at about 25° C. and then is filtered. The resulting solid is suspended in 8-12 V of H₂O at about 25° C., is stirred 10-15 min, and then is filtered. Washing is repeated one more time, and the resulting solid is dried at about 40° C.

### Example 6: Solid Phase Peptide Synthesis of Intermediate Compound 6

Intermediate Compound 6 (SEQ ID NO:12), or a pharmaceutically acceptable salt thereof, can be synthesized by standard SPPS. Briefly, SPPS is conducted using Fmoc-Aib-2-CTC resin (loading factor 0.6-0.9 mmol/g) with the conditions set forth below in Table 6.

TABLE 6
SPPS Conditions for Example 6.

| | | | SPPS Conditions |
|---|---|---|---|
| Cycle | Deprotection | Amino Acid | Solvent for Couplings: DMF |
| 1 | 2 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |
| 2 | 2 × 20-30 min | Fmoc-L-Ala-OH | 2.0 AA/2.2 DIC/2.0 Oxyma |
| | 20% Pip/DMF | | 4 hr, rt |
| 3 | 2 × 20-30 min | Fmoc-L-Lys(t- | 1.5 AA/1.65 DIC/1.5 Oxyma |
| | 20% Pip/DMF | BuOOC—(CH₂)₁₈—COO- | 4 hr, rt |
| | | γ-L-Glu-AEEA) | |
| 4 | 2 × 20-30 min | Fmoc-L-Lys(Boc)-OH | 2.0 AA/2.2 DIC/2.0 Oxyma |
| | 20% Pip/DMF | | 4 hr, rt |
| 5 | 2 × 20-30 min | Fmoc-L-Asp(t-Bu)-OH | 2.0 AA/2.2 DIC/2.0 Oxyma |
| | 20% Pip/DMF | | 4 hr, rt |

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% with TFA/DCM followed by filtrate neutralization is repeated two more times. The resin is washed with 3 V of DCM and is stirred for 10-15 min. All the filtrates and wash are combined, and the resulting mixture cooled to ≤20° C. The fragment solution is concentrated under vacuum to 2-4 V maintaining temperature at ≤20° C. 5 V of ACN is added to the solution, and residual DCM is removed under vacuum (residual DCM concentration ≤15%) maintaining temperature at ≤20° C. An ACN solution of the fragment is added to 5 V of ice-cold H₂O over 2-6 hr (<1 L/min), while maintaining temperature at about 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at about 40° C.

### Example 7: Solid Phase Peptide Synthesis of Intermediate Compound 7

Intermediate Compound 7 (SEQ ID NO:13), or a pharmaceutically acceptable salt thereof, can be synthesized by standard SPPS. Briefly, SPPS is conducted using Fmoc-Gly-2-CTC resin (loading factor 0.6-0.9 mmol/g) with the conditions set forth below in Table 7.

TABLE 7
SPPS Conditions for Example 7.

| | | | SPPS Conditions |
|---|---|---|---|
| Cycle | Deprotection | Amino Acid | Solvent for Couplings: DMF |
| 1 | 2 × 20-30 min | Fmoc-L-Glu(t-Bu)-OH | 2.5 AA/2.5 PyBOP/5.0 DIPEA |
| | 20% Pip/DMF | | 4 hr, rt |
| 2 | 2 × 20-30 min | Fmoc-L-Leu-OH | 2.0 AA/2.2 DIC/2.0 Oxyma |
| | 20% Pip/DMF | | 4-8 hr, rt |
| 3 | 2 × 20-30 min | Fmoc-L-Leu-OH | 2.0 AA/2.2 DIC/2.0 Oxyma |
| | 20% Pip/DMF | | 4-8 hr, rt |
| 4 | 2 × 20-30 min | Fmoc-L-Tyr(t-Bu)-OH | 2.0 AA/2.2 DIC/2.0 Oxyma |
| | 20% Pip/DMF | | 4-8 hr, rt, |
| 5 | 2 × 20-30 min | Fmoc-L-Glu(t-Bu)-OH | 2.0 AA/2.2 DIC/2.0 Oxyma |
| | 20% Pip/DMF | | 4-8 hr, rt |
| 6 | 2 × 20-30 min | Fmoc-L-Ile-OH | 2.0 AA/2.2 DIC/2.0 Oxyma |
| | 20% Pip/DMF | | 4-8 hr, rt |
| 7 | 2 × 20-30 min | Fmoc-L-Phe-OH | 2.0 AA/2.2 DIC/2.0 Oxyma |
| | 20% Pip/DMF | | 4-8 hr, rt |
| 8 | 2 × 20-30 min | Fmoc-L-Ala-OH | 2.0 AA/2.2 DIC/2.0 Oxyma |
| | 20% Pip/DMF | | 4-8 hr, rt |
| 9 | 2 × 20-30 min | Fmoc-Aib-OH | 2.0 AA/2.2 PyBOP/4.0 DIEA |
| | 20% Pip/DMF | | 6-10 hr, rt |
| 10 | 2 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |

Fragment Cleavage and Isolation: Fragment on CTC resin is swelled once using DCM (5 V) for 45 min. 10 V of 1% TFA/DCM is charged to the reactor, and the resulting suspension of the resin is stirred for 10-15 min under nitrogen at 25° C. The filtrate is removed and immediately neutralized by slow addition of a 1.05 equivalent amount of pyridine, and then 5 V of DMSO is added to the filtrate. Resin treatment with 1% TFA/DCM followed by filtrate neutralization is repeated two more times. The resin is washed with 3 V of DCM and is stirred for 10-15 min. All the filtrates and wash are combined. The fragment solution is concentrated under vacuum to 6-10 V maintaining temperature at ≤35° C. (residual DCM concentration ≤15%). DMSO solution of the fragment is added to 11-15 V of H₂O over 2-6 hr (<1 L/min) at about 25° C. The formed slurry of precipitated fragment is stirred for 30-40 min at about 25° C. and then is filtered. The resulting solid is suspended in 8-12 V of H₂O at about 25° C., is stirred 10-15 min, and then is filtered. Washing is repeated one more time, and the resulting solid is dried at about 40° C.

### Example 8: Solid Phase Peptide Synthesis of Intermediate Compound 8

Intermediate Compound 8 (SEQ ID NO:14), or a pharmaceutically acceptable salt thereof, can be synthesized by standard SPPS. Briefly, SPPS is conducted using Fmoc-Ala-2-CTC resin (loading factor 0.6-0.9 mmol/g) with the conditions set forth below in Table 8.

TABLE 8
SPPS Conditions for Example 8.

| | | | SPPS Conditions |
|---|---|---|---|
| Cycle | Deprotection | Amino Acid | Solvent for Couplings: DMF |
| 1 | 2 × 20-30 min | Fmoc-L-Lys(t- | 1.5 AA/1.65 DIC/1.5 Oxyma |
| | 20% Pip/DMF | BuOOC—(CH₂)₁₈—COO- | 4 hr, rt |
| | | γ-L-Glu-AEEA) | |
| 2 | 2 × 20-30 min | Fmoc-L-Lys(Boc)-OH | 2.0 AA/2.2 DIC/2.0 Oxyma |
| | 20% Pip/DMF | | 4 hr, rt |
| 3 | 2 × 20-30 min | Fmoc-L-Asp(t-Bu)-OH | 2.0 AA/2.2 DIC/2.0 Oxyma |
| | 20% Pip/DMF | | 4 hr, rt |

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at 25° C. The filtrate is removed and immediately neutralized by slow addition of a 1.05 equivalent amount of pyridine. Resin treatment with 1% with TFA/DCM followed by filtrate neutralization is repeated two more times. The resin is washed with 3 V of DCM and is stirred for 10-15 min. All the filtrates and wash are combined, and the resulting mixture cooled to ≤20° C. The fragment solution is concentrated under vacuum to 2-4 V maintaining temperature at ≤20° C. 5 V of ACN is added to the solution, and residual DCM is removed under vacuum (residual DCM concentration ≤15%) maintaining temperature at ≤20° C. An ACN solution of the fragment is added to 5 V of ice-cold H₂O over 2-6 hr (<1 L/min), while maintaining temperature at about 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at about 40° C.

### Example 9: Solid Phase Peptide Synthesis of Intermediate Compound 9

Intermediate Compound 9 (SEQ ID NO:15), or a pharmaceutically acceptable salt, can be synthesized by standard SPPS. Briefly, SPPS is conducted using Fmoc-Ala-2-CTC resin (loading factor 0.6-0.9 mmol/g) with the conditions set forth below in Table 9.

TABLE 9
SPPS Conditions for Example 9.

| | | | SPPS Conditions |
|---|---|---|---|
| Cycle | Deprotection | Amino Acid | Solvent for Couplings: DMF |
| 1 | 2 × 20-30 min | Fmoc-L-Lys(t- | 1.5 AA/1.65 DIC/1.5 Oxyma |
| | 20% Pip/DMF | BuOOC—(CH₂)₁₈—COO- | 4 hr, rt |
| | | γ-L-Glu-AEEA) | |
| 2 | 2 × 20-30 min | Fmoc-L-Lys(Boc)-OH | 2.0 AA/2.2 DIC/2.0 Oxyma |
| | 20% Pip/DMF | | 4 hr, rt |
| 3 | 2 × 20-30 min | Fmoc-L-Asp(t-Bu)-OH | 2.0 AA/2.2 DIC/2.0 Oxyma |
| | 20% Pip/DMF | | 4 hr, rt |
| 4 | 2 × 20-30 min | Fmoc-L-Leu-OH | 2.0 AA/2.2 DIC/2.0 Oxyma |
| | 20% Pip/DMF | | 4 hr, rt |
| 5 | 3 × 20-30 min | Fmoc-L-2-Me-Ile-OH | 2.0 AA/2.2 DIC/2.0 Oxyma |
| | 20% Pip/DMF | | 12-18 hr, rt |
| | | | capping procedure |
| 6 | 3 × 20-30 min | Fmoc-L-Ile-OH | 2.0 AA/2.2 DIC/2.0 Oxyma |
| | 20% Pip/DMF | | 12-18 hr, rt |
| | | | capping procedure |
| 7 | 2 × 20-30 min | Fmoc-L-Ser(t-Bu)-OH | 2.0 AA/2.2 DIC/2.0 Oxyma |
| | 20% Pip/DMF | | 4-6 hr, rt |
| 8 | 2 × 20-30 min | Fmoc-L-Tyr(t-Bu)-OH | 2.0 AA/2.2 DIC/2.0 Oxyma |
| | 20% Pip/DMF | | 4-6 hr, rt |
| 9 | 2 × 20-30 min | Fmoc-L-Asp(t-Bu)-OH | 2.0 AA/2.2 DIC/2.0 Oxyma |
| | 20% Pip/DMF | | 4-6 hr, rt |
| 10 | 2 × 10 min | Fmoc-L-Ser(t-Bu)-OH | 2.0 AA/2.2 DIC/2.0 Oxyma |
| | 5% Oxyma/20% | | 4-6 hr, rt |
| | Pip/DMF | | |
| 11 | 2 × 10 min | Fmoc-L-Thr(t-Bu)-OH | 2.0 AA/2.2 DIC/2.0 Oxyma |
| | 5% Oxyma/20% | | 4-6 hr, rt |
| | Pip/DMF | | |
| 12 | 2 × 10 min | Fmoc-L-Phe(t-Bu)-OH | 2.0 AA/2.2 DIC/2.0 Oxyma |
| | 5% Oxyma/20% | | 4-6 hr, rt |
| | Pip/DMF | | |
| 13 | 2 × 10 min | Fmoc-L-Thr(t-Bu)-OH | 2.0 AA/2.2 DIC/2.2 Oxyma |
| | 5% Oxyma/20% | | 4-6 hr, rt |
| | Pip/DMF | | |
| 14 | 2 × 10 min | Boc-L-Tyr(t-Bu)- | 1.5 AA/1.65 DIC/1.5 Oxyma |
| | 5% Oxyma/20% | Aib-L-Gln(Trt)- | 4-6 hr, rt |
| | Pip/DMF | Gly-OH | |

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at 25° C. The filtrate is removed and immediately neutralized by slow addition of a 1.05 equivalent amount of pyridine. Resin treatment with 1% with TFA/DCM followed by filtrate neutralization is repeated two more times. The resin is washed with 3 V of DCM and is stirred for 10-15 min. All the filtrates and wash are combined, and the resulting mixture cooled to ≤20° C. The fragment solution is concentrated under vacuum to 2-4 V maintaining temperature at ≤20° C. 5 V of ACN is added to the solution, and residual DCM is removed under vacuum (residual DCM concentration ≤15%) maintaining temperature at ≤20° C. An ACN solution of the fragment is added to 5 V of ice-cold H₂O over 2-6 hr (<1 L/min), while maintaining temperature at about 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at about 40° C.

### Example 10: Solid Phase Peptide Synthesis of Intermediate Compound 10

Intermediate Compound 10 (SEQ ID NO:16), or a pharmaceutically acceptable salt thereof, can be synthesized by standard SPPS. Briefly, SPPS is conducted using Sieber resin (loading factor 0.6-0.9 mmol/g) with the conditions set forth below in Table 10.

TABLE 10
SPPS Conditions for Example 10.

| | | | SPPS Conditions |
|---|---|---|---|
| Cycle | Deprotection | Amino Acid | Solvent for Couplings: DMF |
| 1 | 2 × 30 min | Fmoc-L-Ser(t-Bu)-OH | 2.0 AA/2.2 DIC/2.0 Oxyma |
| | 20% Pip/DMF | | 4 hr, rt |
| 2 | 2 × 30 min | Fmoc-L-Pro-OH | 2.0 AA/2.2 DIC/2.0 Oxyma |
| | 20% Pip/DMF | | 6 hr, rt |
| 3 | 2 × 30 min | Fmoc-L-Pro-OH | 2.0 AA/2.2 DIC/2.0 Oxyma |
| | 20% Pip/DMF | | 6 hr, rt |
| 4 | 2 × 30 min | Fmoc-L-Pro-OH | 2.0 AA/2.2 DIC/2.0 Oxyma |
| | 20% Pip/DMF | | 6 hr, rt |
| 3-4* | Alternatively use coupling of Fmoc-L-Pro-Pro-OH dimer instead of step 3 & 4 | | |
| 5 | 2 × 30 min | Fmoc-L-Ala-OH | 2.0 AA/2.2 DIC/2.0 Oxyma |
| | 20% Pip/DMF | | 4 hr, rt |
| 6 | 2 × 30 min | Fmoc-Gly-OH | 2.0 AA/2.2 DIC/2.0 Oxyma |
| | 20% Pip/DMF | | 4 hr, rt |
| 7 | 2 × 30 min | Fmoc-L-Ser(t-Bu)-OH | 2.0 AA/2.2 DIC/2.0 Oxyma |
| | 20% Pip/DMF | | 4 hr, rt |
| 8 | 2 × 30 min | Fmoc-L-Ser(t-Bu)-OH | 2.0 AA/2.2 DIC/2.0 Oxyma |
| | 20% Pip/DMF | | 4 hr, rt |
| 9 | 2 × 30 min | Fmoc-L-Pro-OH | 2.0 AA/2.2 DIC/2.0 Oxyma |
| | 20% Pip/DMF | | 4 hr, rt |
| 10 | 2 × 30 min | Fmoc-Gly-OH | 2.0 AA/2.2 DIC/2.0 Oxyma |
| | 20% Pip/DMF | | 4 hr, rt |
| 11 | 2 × 30 min | Fmoc-Gly-OH | 2.0 AA/2.2 DIC/2.0 Oxyma |
| | 20% Pip/DMF | | 4 hr, rt |
| 10-11* | Alternatively use coupling of Fmoc-Gly-Gly-OH dimer instead of step 3 & 4 | | |
| 12 | 2 × 20-30 min | Fmoc-L-Glu(t-Bu)-OH | 2.5 AA/2.5 PyBOP/5.0 DIPEA |
| | 20% Pip/DMF | | 4 hr, rt |
| 13 | 2 × 20-30 min | Fmoc-L-Leu-OH | 2.0 AA/2.2 DIC/2.0 Oxyma |
| | 20% Pip/DMF | | 4-8 hr, rt |
| 14 | 2 × 20-30 min | Fmoc-L-Leu-OH | 2.0 AA/2.2 DIC/2.0 Oxyma |
| | 20% Pip/DMF | | 4-8 hr, rt |
| 15 | 2 × 20-30 min | Fmoc-L-Tyr(t-Bu)-OH | 2.0 AA/2.2 DIC/2.0 Oxyma |
| | 20% Pip/DMF | | 4-8 hr, rt |
| 16 | 2 × 20-30 min | Fmoc-L-Glu(t-Bu)-OH | 2.0 AA/2.2 DIC/2.0 Oxyma |
| | 20% Pip/DMF | | 4-8 hr, rt |
| 17 | 2 × 20-30 min | Fmoc-L-Ile-OH | 2.0 AA/2.2 DIC/2.0 Oxyma |
| | 20% Pip/DMF | | 4-8 hr, rt |
| 18 | 2 × 20-30 min | Fmoc-L-Phe-OH | 2.0 AA/2.2 DIC/2.0 Oxyma |
| | 20% Pip/DMF | | 4-8 hr, rt |

Fmoc Deprotection, Fragment Cleavage and Isolation: Fragment on Sieber resin is stirred twice with 10 V of 20% piperidine/DMF for 20-30 min, then washed six times with 10 V of DMF. The de-Fmoced fragment on Sieber resin is swelled twice using 10 V DCM for 10-20 min. Reactor with resin is cooled to about 15° C. 20 V of 5% TFA/DCM is charged to the reactor and is stirred for 2 hr under nitrogen maintaining temperature at about 15° C. The resin is filtered and is washed with 3×10 V of DCM. All the filtrates are combined together. DCM is removed from the resulting solution under reduced pressure while maintaining internal temperature at ≤20° C. to 22.5 V residual volume. MTBE (25 V) is charged to the solution and DCM/MTBE solvents are again removed under reduced pressure while maintaining temperature at ≤20° C. to 22.5 V residual volume. Addition of MTBE/distillation operation is repeated until residual concentration of fragment in supernatant does not reached <0.11 wt %. Then, the resulting slurry is filtered while maintaining temperature at about 15° C. To the cake, 14 V of fresh MTBE is added, and the slurry is stirred for 30 min at about 15° C. and then is filtered. Washing is repeated one more time, and the resulting solid is dried at about 35° C.

### Example 11: Solid Phase Peptide Synthesis of Intermediate Compound 11

Intermediate Compound 11 (SEQ ID NO:17), or a pharmaceutically acceptable salt thereof, can be synthesized by standard SPPS. Briefly, SPPS is conducted using Fmoc-Aib-2-CTC resin (0.6-0.9 mmol/g) with the conditions set forth below in Table 11.

TABLE 11
SPPS Conditions for Example 11.

| | | | SPPS Conditions |
|---|---|---|---|
| Cycle | Deprotection | Amino Acid | Solvent for Couplings: DMF |
| 1 | 2 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |
| 2 | 2 × 20-30 min | Fmoc-L-Ala-OH | 2.0 AA/2.2 DIC/2.0 Oxyma |
| | 20% Pip/DMF | | 4 hr, rt |
| 3 | 2 × 20-30 min | Fmoc-L-Lys(t- | 1.5 AA/1.65 DIC/1.5 Oxyma |
| | 20% Pip/DMF | BuOOC—(CH₂)₁₈—COO- | 4 hr, rt |
| | | γ-L-Glu-AEEA) | |
| 4 | 2 × 20-30 min | Fmoc-L-Lys(Boc)-OH | 2.0 AA/2.2 DIC/2.0 Oxyma |
| | 20% Pip/DMF | | 4 hr, rt |
| 5 | 2 × 20-30 min | Fmoc-L-Asp(t-Bu)-OH | 2.0 AA/2.2 DIC/2.0 Oxyma |
| | 20% Pip/DMF | | 4 hr, rt |

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% with TFA/DCM followed by filtrate neutralization is repeated two more times. The resin is washed with 3 V of DCM and is stirred for 10-15 min. All the filtrates and wash are combined, and the resulting mixture cooled to ≤20° C. The fragment solution is concentrated under vacuum to 2-4 V maintaining temperature at ≤20° C. 5 V of ACN is added to the solution, and residual DCM is removed under vacuum (residual DCM concentration ≤X %) maintaining temperature at ≤20° C. An ACN solution of the fragment is added to 5 V of ice-cold H₂O over 2-6 hr (<1 L/min), while maintaining temperature at about 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at about 40° C.

### Example 12: Solid Phase Peptide Synthesis of Intermediate Compound 12

Intermediate Compound 12 (SEQ ID NO:18), or a pharmaceutically acceptable salt thereof, can be synthesized by standard SPPS. Briefly, SPPS is conducted using Sieber resin (loading factor 0.6-0.9 mmol/g) with the conditions set forth below in Table 12.

TABLE 12
SPPS Conditions for Example 12.

| | | | SPPS Conditions |
|---|---|---|---|
| Cycle | Deprotection | Amino Acid | Solvent for Couplings: DMF |
| 1 | 2 × 30 min | Fmoc-L-Ser(t-Bu)-OH | 2.0 AA/2.2 DIC/2.0 Oxyma |
| | 20% Pip/DMF | | 4 hr, rt |
| 2 | 2 × 30 min | Fmoc-L-Pro-OH | 2.0 AA/2.2 DIC/2.0 Oxyma |
| | 20% Pip/DMF | | 6 hr, rt |
| 3 | 2 × 30 min | Fmoc-L-Pro-OH | 2.0 AA/2.2 DIC/2.0 Oxyma |
| | 20% Pip/DMF | | 6 hr, rt |
| 4 | 2 × 30 min | Fmoc-L-Pro-OH | 2.0 AA/2.2 DIC/2.0 Oxyma |
| | 20% Pip/DMF | | 6 hr, rt |
| 3-4* | Alternatively use coupling of Fmoc-L-Pro-Pro-OH dimer instead of step 3 & 4 | | |
| 5 | 2 × 30 min | Fmoc-L-Ala-OH | 2.0 AA/2.2 DIC/2.0 Oxyma |
| | 20% Pip/DMF | | 4 hr, rt |
| 6 | 2 × 30 min | Fmoc-Gly-OH | 2.0 AA/2.2 DIC/2.0 Oxyma |
| | 20% Pip/DMF | | 4 hr, rt |
| 7 | 2 × 30 min | Fmoc-L-Ser(t-Bu)-OH | 2.0 AA/2.2 DIC/2.0 Oxyma |
| | 20% Pip/DMF | | 4 hr, rt |
| 8 | 2 × 30 min | Fmoc-L-Ser(t-Bu)-OH | 2.0 AA/2.2 DIC/2.0 Oxyma |
| | 20% Pip/DMF | | 4 hr, rt |
| 9 | 2 × 30 min | Fmoc-L-Pro-OH | 2.0 AA/2.2 DIC/2.0 Oxyma |
| | 20% Pip/DMF | | 4 hr, rt |
| 10 | 2 × 30 min | Fmoc-Gly-OH | 2.0 AA/2.2 DIC/2.0 Oxyma |
| | 20% Pip/DMF | | 4 hr, rt |
| 11 | 2 × 30 min | Fmoc-Gly-OH | 2.0 AA/2.2 DIC/2.0 Oxyma |
| | 20% Pip/DMF | | 4 hr, rt |
| 10-11* | Alternatively use coupling of Fmoc-Gly-Gly-OH dimer instead of step 3 & 4 | | |
| 12 | 2 × 20-30 min | Fmoc-L-Glu(t-Bu)-OH | 2.5 AA/2.5 PyBOP/5.0 DIPEA |
| | 20% Pip/DMF | | 4 hr, rt |
| 13 | 2 × 20-30 min | Fmoc-L-Leu-OH | 2.0 AA/2.2 DIC/2.0 Oxyma |
| | 20% Pip/DMF | | 4-8 hr, rt |
| 14 | 2 × 20-30 min | Fmoc-L-Leu-OH | 2.0 AA/2.2 DIC/2.0 Oxyma |
| | 20% Pip/DMF | | 4-8 hr, rt |
| 15 | 2 × 20-30 min | Fmoc-L-Tyr(t-Bu)-OH | 2.0 AA/2.2 DIC/2.0 Oxyma |
| | 20% Pip/DMF | | 4-8 hr, rt |
| 16 | 2 × 20-30 min | Fmoc-L-Glu(t-Bu)-OH | 2.0 AA/2.2 DIC/2.0 Oxyma |
| | 20% Pip/DMF | | 4-8 hr, rt |
| 17 | 2 × 20-30 min | Fmoc-L-Ile-OH | 2.0 AA/2.2 DIC/2.0 Oxyma |
| | 20% Pip/DMF | | 4-8 hr, rt |
| 18 | 2 × 20-30 min | Fmoc-L-Phe-OH | 2.0 AA/2.2 DIC/2.0 Oxyma |
| | 20% Pip/DMF | | 4-8 hr, rt |
| 19 | 2 × 20-30 min | Fmoc-L-Ala-OH | 2.0 AA/2.2 DIC/2.0 Oxyma |
| | 20% Pip/DMF | | 4-8 hr, rt |

Fmoc Deprotection, Fragment Cleavage and Isolation: Fragment on Sieber resin is stirred twice with 10 V of 20% piperidine/DMF for 20-30 min, then washed six times with 10 V of DMF. The de-Fmoced fragment on Sieber resin is swelled twice using 10 V DCM for 10-20 min. Reactor with resin is cooled to about 15° C. 20 V of 5% TFA/DCM is charged to the reactor and is stirred for 2 hr under nitrogen maintaining temperature at about 15° C. The resin is filtered and is washed with 3×10 V of DCM. All the filtrates are combined together. DCM is removed from the resulting solution under reduced pressure while maintaining internal temperature at ≤20° C. to 22.5 V residual volume. MTBE (25 V) is charged to the solution, and DCM/MTBE solvents are again removed under reduced pressure while maintaining temperature at ≤20° C. to 22.5 V residual volume. Addition of MTBE/distillation operation is repeated until residual concentration of fragment in supernatant has not reached <0.11 wt %. Then, the resulting slurry is filtered while maintaining temperature at about 15° C. To the cake, 14 V of fresh MTBE is added, and the slurry is stirred for 30 min at about 15° C. and then is filtered. Washing is repeated one more time, and the resulting solid is dried at about 35° C.

### Example 13: Solid Phase Peptide Synthesis of Intermediate Compound 13

Intermediate Compound 13 (SEQ ID NO:19), or a pharmaceutically acceptable salt thereof, can be synthesized by standard SPPS. Briefly, SPPS is conducted using Sieber resin (loading factor 0.6-0.9 mmol/g) with the conditions set forth below in Table 13.

TABLE 13
SPPS Conditions for Example 13.

| | | | SPPS Conditions |
|---|---|---|---|
| Cycle | Deprotection | Amino Acid | Solvent for Couplings: DMF |
| 1 | 2 × 30 min | Fmoc-L-Ser(t-Bu)-OH | 2.0 AA/2.2 DIC/2.0 Oxyma |
| | 20% Pip/DMF | | 4 hr, rt |
| 2 | 2 × 30 min | Fmoc-L-Pro-OH | 2.0 AA/2.2 DIC/2.0 Oxyma |
| | 20% Pip/DMF | | 6 hr, rt |
| 3 | 2 × 30 min | Fmoc-L-Pro-OH | 2.0 AA/2.2 DIC/2.0 Oxyma |
| | 20% Pip/DMF | | 6 hr, rt |
| 4 | 2 × 30 min | Fmoc-L-Pro-OH | 2.0 AA/2.2 DIC/2.0 Oxyma |
| | 20% Pip/DMF | | 6 hr, rt |
| 3-4* | Alternatively use coupling of Fmoc-L-Pro-Pro-OH dimer instead of step 3 & 4 | | |
| 5 | 2 × 30 min | Fmoc-L-Ala-OH | 2.0 AA/2.2 DIC/2.0 Oxyma |
| | 20% Pip/DMF | | 4 hr, rt |
| 6 | 2 × 30 min | Fmoc-Gly-OH | 2.0 AA/2.2 DIC/2.0 Oxyma |
| | 20% Pip/DMF | | 4 hr, rt |
| 7 | 2 × 30 min | Fmoc-L-Ser(t-Bu)-OH | 2.0 AA/2.2 DIC/2.0 Oxyma |
| | 20% Pip/DMF | | 4 hr, rt |
| 8 | 2 × 30 min | Fmoc-L-Ser(t-Bu)-OH | 2.0 AA/2.2 DIC/2.0 Oxyma |
| | 20% Pip/DMF | | 4 hr, rt |
| 9 | 2 × 30 min | Fmoc-L-Pro-OH | 2.0 AA/2.2 DIC/2.0 Oxyma |
| | 20% Pip/DMF | | 4 hr, rt |
| 10 | 2 × 30 min | Fmoc-Gly-OH | 2.0 AA/2.2 DIC/2.0 Oxyma |
| | 20% Pip/DMF | | 4 hr, rt |
| 11 | 2 × 30 min | Fmoc-Gly-OH | 2.0 AA/2.2 DIC/2.0 Oxyma |
| | 20% Pip/DMF | | 4 hr, rt |
| 10-11* | Alternatively use coupling of Fmoc-Gly-Gly-OH dimer instead of step 3 & 4 | | |
| 12 | 2 × 20-30 min | Fmoc-L-Glu(t-Bu)-OH | 2.5 AA/2.5 PyBOP/5.0 DIPEA |
| | 20% Pip/DMF | | 4 hr, rt |
| 13 | 2 × 20-30 min | Fmoc-L-Leu-OH | 2.0 AA/2.2 DIC/2.0 Oxyma |
| | 20% Pip/DMF | | 4-8 hr, rt |
| 14 | 2 × 20-30 min | Fmoc-L-Leu-OH | 2.0 AA/2.2 DIC/2.0 Oxyma |
| | 20% Pip/DMF | | 4-8 hr, rt |
| 15 | 2 × 20-30 min | Fmoc-L-Tyr(t-Bu)-OH | 2.0 AA/2.2 DIC/2.0 Oxyma |
| | 20% Pip/DMF | | 4-8 hr, rt |
| 16 | 2 × 20-30 min | Fmoc-L-Glu(t-Bu)-OH | 2.0 AA/2.2 DIC/2.0 Oxyma |
| | 20% Pip/DMF | | 4-8 hr, rt |
| 17 | 2 × 20-30 min | Fmoc-L-Ile-OH | 2.0 AA/2.2 DIC/2.0 Oxyma |
| | 20% Pip/DMF | | 4-8 hr, rt |
| 18 | 2 × 20-30 min | Fmoc-L-Phe-OH | 2.0 AA/2.2 DIC/2.0 Oxyma |
| | 20% Pip/DMF | | 4-8 hr, rt |
| 19 | 2 × 20-30 min | Fmoc-L-Ala-OH | 2.0 AA/2.2 DIC/2.0 Oxyma |
| | 20% Pip/DMF | | 4-8 hr, rt |
| 20 | 3 × 20-30 min | Fmoc-Aib-OH | 2.0 AA/2.2 DIC/2.0 Oxyma |
| | 20% Pip/DMF | | 8-12 hr, rt |
| | | | capping procedure |
| 21 | 3 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |

Fmoc Deprotection, Fragment Cleavage and Isolation: Fragment on Sieber resin is stirred twice with 10 V of 20% piperidine/DMF for 20-30 min, then washed six times with 10 V of DMF. The de-Fmoced fragment on Sieber resin is swelled twice using 10 V DCM for 10-20 min. Reactor with resin is cooled to about 15° C. 20 V of 5% TFA/DCM is charged to the reactor and is stirred for 2 hr under nitrogen maintaining temperature at about 15° C. The resin is filtered and is washed with 3×10 V of DCM. All the filtrates are combined together. DCM is removed from the resulting solution under reduced pressure while maintaining temperature at ≤20° C. to 22.5 V residual volume. MTBE (25 V) is charged to the solution, and DCM/MTBE solvents are again removed under reduced pressure while maintaining temperature at ≤20° C. to 22.5 V residual volume. Addition of MTBE/distillation operation is repeated until residual concentration of fragment in supernatant has not reached <0.11 wt %. Then, the resulting slurry is filtered while maintaining temperature at about 15° C. To the cake, 14 V of fresh MTBE is added, and the slurry is stirred for 30 min at about 15° C. and then is filtered. Washing is repeated one more time, and the resulting solid is dried at about 35° C.

### Example 14: Solid Phase Peptide Synthesis of Intermediate Compound 14

Intermediate Compound 14 (SEQ ID NO:20), or a pharmaceutically acceptable salt thereof, can be synthesized by standard SPPS. Briefly, SPPS is conducted using Fmoc-Aib-2-CTC resin (loading factor 0.6-0.9 mmol/g) with the conditions set forth below in Table 14.

TABLE 14
SPPS Conditions for Example 14.

| | | | SPPS Conditions |
|---|---|---|---|
| Cycle | Deprotection | Amino Acid | Solvent for Couplings: DMF |
| 1 | 2 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |
| 2 | 2 × 20-30 min | Fmoc-L-Ala-OH | 2.0 AA/2.2 DIC/2.0 Oxyma |
| | 20% Pip/DMF | | 4 hr, rt |
| 3 | 2 × 20-30 min | Fmoc-L-Lys(t- | 1.5 AA/1.65 DIC/1.5 Oxyma |
| | 20% Pip/DMF | BuOOC—(CH₂)₁₈—COO- | 4 hr, rt |
| | | γ-L-Glu-AEEA) | |
| 4 | 2 × 20-30 min | Fmoc-L-Lys(Boc)-OH | 2.0 AA/2.2 DIC/2.0 Oxyma |
| | 20% Pip/DMF | | 4 hr, rt |
| 5 | 2 × 20-30 min | Fmoc-L-Asp(t-Bu)-OH | 2.0 AA/2.2 DIC/2.0 Oxyma |
| | 20% Pip/DMF | | 4 hr, rt |
| 6 | 2 × 20-30 min | Fmoc-L-Leu-OH | 2.0 AA/2.2 DIC/2.0 Oxyma |
| | 20% Pip/DMF | | 4 hr, rt |
| 7 | 3 × 20-30 min | Fmoc-L-2-Me-Ile-OH | 2.0 AA/2.2 DIC/2.0 Oxyma |
| | 20% Pip/DMF | | 12-18 hr, rt |
| | | | capping procedure |
| 8 | 3 × 20-30 min | Fmoc-L-Ile-OH | 2.0 AA/2.2 DIC/2.0 Oxyma |
| | 20% Pip/DMF | | 12-18 hr, rt |
| | | | capping procedure |
| 9 | 2 × 20-30 min | Fmoc-L-Ser(t-Bu)-OH | 2.0 AA/2.2 DIC/2.0 Oxyma |
| | 20% Pip/DMF | | 4-6 hr, rt |
| 10 | 2 × 20-30 min | Fmoc-L-Tyr(t-Bu)-OH | 2.0 AA/2.2 DIC/2.0 Oxyma |
| | 20% Pip/DMF | | 4-6 hr, rt |
| 11 | 2 × 20-30 min | Fmoc-L-Asp(t-Bu)-OH | 2.0 AA/2.2 DIC/2.0 Oxyma |
| | 20% Pip/DMF | | 4-6 hr, rt |
| 12 | 2 × 10 min | Fmoc-L-Ser(t-Bu)-OH | 2.0 AA/2.2 DIC/2.0 Oxyma |
| | 5% Oxyma/20% | | 4-6 hr, rt |
| | Pip/DMF | | |
| 13 | 2 × 10 min | Fmoc-L-Thr(t-Bu)-OH | 2.0 AA/2.2 DIC/2.0 Oxyma |
| | 5% Oxyma/20% | | 4-6 hr, rt |
| | Pip/DMF | | |
| 14 | 2 × 10 min | Fmoc-L-Phe(t-Bu)-OH | 2.0 AA/2.2 DIC/2.0 Oxyma |
| | 5% Oxyma/20% | | 4-6 hr, rt |
| | Pip/DMF | | |
| 15 | 2 × 10 min | Fmoc-L-Thr(t-Bu)-OH | 2.0 AA/2.2 DIC/2.2 Oxyma |
| | 5% Oxyma/20% | | 4-6 hr, rt |
| | Pip/DMF | | |
| 16 | 2 × 10 min | Boc-L-Tyr(t-Bu)- | 1.5 AA/1.65 DIC/1.5 Oxyma |
| | 5% Oxyma/20% | Aib-L-Gln(Trt)- | 4-6 hr, rt |
| | Pip/DMF | Gly-OH | |

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% with TFA/DCM followed by filtrate neutralization is repeated two more times. The resin is washed with 3 V of DCM and is stirred for 10-15 min. All the filtrates and wash are combined, and the resulting mixture cooled to ≤20° C. The fragment solution is concentrated under vacuum to 2-4 V maintaining temperature at ≤20° C. 5 V of ACN is added to the solution, and residual DCM is removed under vacuum (residual DCM concentration ≤X %) maintaining temperature at ≤20° C. An ACN solution of the fragment is added to 5 V of ice-cold H₂O over 2-6 hr (<1 L/min), while maintaining temperature at about 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at about 40° C.

### Example 15: Solid Phase Peptide Synthesis of Intermediate Compound 15

Intermediate Compound (SEQ ID NO:21), or a pharmaceutically acceptable salt thereof, can be synthesized by standard SPPS. Briefly, SPPS is conducted using Fmoc-Aib-2-CTC resin (loading factor 0.6-0.9 mmol/g) with the conditions set forth below in Table 15.

TABLE 15
SPPS Conditions for Example 15.

| | | | SPPS Conditions |
|---|---|---|---|
| Cycle | Deprotection | Amino Acid | Solvent for Couplings: DMF |
| 1 | 2 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |
| 2 | 2 × 20-30 min | Fmoc-L-Ala-OH | 2.0 AA/2.2 DIC/2.0 Oxyma |
| | 20% Pip/DMF | | 4 hr, rt |
| 3 | 2 × 20-30 min | Fmoc-L-Lys(PG)-OH | 2.0 AA/2.2 DIC/2.0 Oxyma |
| | 20% Pip/DMF | | 6-8 hr, rt |
| 4 | 2 × 20-30 min | Fmoc-L-Lys(Boc)-OH | 2.0 AA/2.2 DIC/2.0 Oxyma |
| | 20% Pip/DMF | | 4 hr, rt |
| 5 | 2 × 20-30 min | Fmoc-L-Asp(t-Bu)-OH | 2.0 AA/2.2 DIC/2.0 Oxyma |
| | 20% Pip/DMF | | 4 hr, rt |
| 6 | 2 × 20-30 min | Fmoc-L-Leu-OH | 2.0 AA/2.2 DIC/2.0 Oxyma |
| | 20% Pip/DMF | | 4 hr, rt |
| 7 | 3 × 20-30 min | Fmoc-L-2-Me-Ile-OH | 2.0 AA/2.2 DIC/2.0 Oxyma |
| | 20% Pip/DMF | | 12-18 hr, rt |
| | | | capping procedure |
| 8 | 3 × 20-30 min | Fmoc-L-Ile-OH | 2.0 AA/2.2 DIC/2.0 Oxyma |
| | 20% Pip/DMF | | 12-18 hr, rt |
| | | | capping procedure |
| 9 | 2 × 20-30 min | Fmoc-L-Ser(t-Bu)-OH | 2.0 AA/2.2 DIC/2.0 Oxyma |
| | 20% Pip/DMF | | 4-6 hr, rt |
| 10 | 2 × 20-30 min | Fmoc-L-Tyr(t-Bu)-OH | 2.0 AA/2.2 DIC/2.0 Oxyma |
| | 20% Pip/DMF | | 4-6 hr, rt |
| 11 | 2 × 20-30 min | Fmoc-L-Asp(t-Bu)-OH | 2.0 AA/2.2 DIC/2.0 Oxyma |
| | 20% Pip/DMF | | 4-6 hr, rt |
| 12 | 2 × 10 min | Fmoc-L-Ser(t-Bu)-OH | 2.0 AA/2.2 DIC/2.0 Oxyma |
| | 5% Oxyma/20% | | 4-6 hr, rt |
| | Pip/DMF | | |
| 13 | 2 × 10 min | Fmoc-L-Thr(t-Bu)-OH | 2.0 AA/2.2 DIC/2.0 Oxyma |
| | 5% Oxyma/20% | | 4-6 hr, rt |
| | Pip/DMF | | |
| 14 | 2 × 10 min | Fmoc-L-Phe(t-Bu)-OH | 2.0 AA/2.2 DIC/2.0 Oxyma |
| | 5% Oxyma/20% | | 4-6 hr, rt |
| | Pip/DMF | | |
| 15 | 2 × 10 min | Fmoc-L-Thr(t-Bu)-OH | 2.0 AA/2.2 DIC/2.2 Oxyma |
| | 5% Oxyma/20% | | 4-6 hr, rt |
| | Pip/DMF | | |
| 16 | 2 × 10 min | Boc-L-Tyr(t-Bu)- | 1.5 AA/1.65 DIC/1.5 Oxyma |
| | 5% Oxyma/20% | Aib-L-Gln(Trt)- | 4-6 hr, rt |
| | Pip/DMF | Gly-OH | |

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% with TFA/DCM followed by filtrate neutralization is repeated two more times. The resin is washed with 3 V of DCM and is stirred for 10-15 min. All the filtrates and wash are combined, and the resulting mixture cooled to ≤20° C. The fragment solution is concentrated under vacuum to 2-4 V maintaining temperature at ≤20° C. 5 V of ACN is added to the solution, and residual DCM is removed under vacuum (residual DCM concentration ≤X %) maintaining temperature at ≤20° C. An ACN solution of the fragment is added to 5 V of ice-cold H₂O over 2-6 hr (<1 L/min), while maintaining temperature at about 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at about 40° C.

### Example 16: Solid Phase Peptide Synthesis of Intermediate Compound 16

Intermediate Compound 16 (SEQ ID NO:22), or a pharmaceutically acceptable salt thereof, can be synthesized by standard SPPS. Briefly, SPPS is conducted using Fmoc-Ala-2-CTC resin (loading factor 0.6-0.9 mmol/g) with the conditions set forth below in Table 16.

TABLE 16
SPPS Conditions for Example 16.

| | | | SPPS Conditions |
|---|---|---|---|
| Cycle | Deprotection | Amino Acid | Solvent for Couplings: DMF |
| 1 | 2 × 20-30 min | Fmoc-L-Lys(PG)-OH | 2.0 AA/2.2 DIC/2.0 Oxyma |
| | 20% Pip/DMF | | 4 hr, rt |
| 2 | 2 × 20-30 min | Fmoc-L-Lys(Boc)-OH | 2.0 AA/2.2 DIC/2.0 Oxyma |
| | 20% Pip/DMF | | 4 hr, rt |
| 3 | 2 × 20-30 min | Fmoc-L-Asp(t-Bu)-OH | 2.0 AA/2.2 DIC/2.0 Oxyma |
| | 20% Pip/DMF | | 4 hr, rt |
| 4 | 2 × 20-30 min | Fmoc-L-Leu-OH | 2.0 AA/2.2 DIC/2.0 Oxyma |
| | 20% Pip/DMF | | 4 hr, rt |
| 5 | 3 × 20-30 min | Fmoc-L-2-Me-Ile-OH | 2.0 AA/2.2 DIC/2.0 Oxyma |
| | 20% Pip/DMF | | 12-18 hr, rt |
| | | | capping procedure |
| 6 | 3 × 20-30 min | Fmoc-L-Ile-OH | 2.0 AA/2.2 DIC/2.0 Oxyma |
| | 20% Pip/DMF | | 12-18 hr, rt |
| | | | capping procedure |
| 7 | 2 × 20-30 min | Fmoc-L-Ser(t-Bu)-OH | 2.0 AA/2.2 DIC/2.0 Oxyma |
| | 20% Pip/DMF | | 4-6 hr, rt |
| 8 | 2 × 20-30 min | Fmoc-L-Tyr(t-Bu)-OH | 2.0 AA/2.2 DIC/2.0 Oxyma |
| | 20% Pip/DMF | | 4-6 hr, rt |
| 9 | 2 × 20-30 min | Fmoc-L-Asp(t-Bu)-OH | 2.0 AA/2.2 DIC/2.0 Oxyma |
| | 20% Pip/DMF | | 4-6 hr, rt |
| 10 | 2 × 10 min | Fmoc-L-Ser(t-Bu)-OH | 2.0 AA/2.2 DIC/2.0 Oxyma |
| | 5% Oxyma/20% | | 4-6 hr, rt |
| | Pip/DMF | | |
| 11 | 2 × 10 min | Fmoc-L-Thr(t-Bu)-OH | 2.0 AA/2.2 DIC/2.0 Oxyma |
| | 5% Oxyma/20% | | 4-6 hr, rt |
| | Pip/DMF | | |
| 12 | 2 × 10 min | Fmoc-L-Phe(t-Bu)-OH | 2.0 AA/2.2 DIC/2.0 Oxyma |
| | 5% Oxyma/20% | | 4-6 hr, rt |
| | Pip/DMF | | |
| 13 | 2 × 10 min | Fmoc-L-Thr(t-Bu)-OH | 2.0 AA/2.2 DIC/2.2 Oxyma |
| | 5% Oxyma/20% | | 4-6 hr, rt |
| | Pip/DMF | | |
| 14 | 2 × 10 min | Boc-L-Tyr(t-Bu)- | 1.5 AA/1.65 DIC/1.5 Oxyma |
| | 5% Oxyma/20% | Aib-L-Gln(Trt)- | 4-6 hr, rt |
| | Pip/DMF | Gly-OH | |

Fragment Cleavage and Isolation: Fragment on CTC resin is swelled once using DCM (5 V) for 45 min. 5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% with TFA/DCM followed by filtrate neutralization is repeated two more times. The Resin is washed with 3 V of DCM and is stirred for 10-15 min. All the filtrates and wash are combined, and the resulting mixture cooled to ≤20° C. The fragment solution is concentrated under vacuum to 2-4 V maintaining temperature at ≤20° C. 5 V of ACN is added to the solution, and residual DCM is removed under vacuum (residual DCM concentration ≤X %) maintaining temperature at ≤20° C. An ACN solution of the fragment is added to 5 V of ice-cold H₂O over 2-6 hr (<1 L/min), while maintaining temperature at 0° C. The resulting slurry of precipitated fragment is stirred for 30-40 min at about 0° C. and then is filtered at about 0° C. The resulting solid is suspended in 3-5 V of H₂O at about 25° C., is stirred 10-15 min, and then is filtered. Washing is repeated one more time, and the resulting solid is dried at about 40° C.

### Example 17: Hybrid Liquid Solid Phase Peptide Synthesis of Intermediate Compound 17

Intermediate Compound 17 (SEQ ID NO:23), or a pharmaceutically acceptable salt thereof, can be synthesized by standard SPPS. Briefly, SPPS is conducted using Fmoc-Aib-2-CTC-hydrazine resin (loading factor 0.6-0.9 mmol/g) with the conditions set forth below in Table 17.

TABLE 17
SPPS Conditions for Example 17.

| | | | SPPS Conditions |
|---|---|---|---|
| Cycle | Deprotection | Amino Acid | Solvent for Couplings: DMF |
| 1 | 2 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |
| 2 | 2 × 20-30 min | Fmoc-L-Ala-OH | 2.0 AA/2.2 DIC/2.0 Oxyma |
| | 20% Pip/DMF | | 4 h, rt |
| 3 | 2 × 20-30 min | Fmoc-L-Lys(t- | 1.5 AA/1.65 DIC/1.5 Oxyma |
| | 20% Pip/DMF | BuOOC—(CH₂)₁₈—COO- | 4 hr, rt |
| | | γ-L-Glu-AEEA) | |
| 4 | 2 × 20-30 min | Fmoc-L-Lys(Boc)-OH | 2.0 AA/2.2 DIC/2.0 Oxyma |
| | 20% Pip/DMF | | 4 hr, rt |
| 5 | 2 × 20-30 min | Fmoc-L-Asp(t-Bu)-OH | 2.0 AA/2.2 DIC/2.0 Oxyma |
| | 20% Pip/DMF | | 4 hr, rt |
| 6 | 2 × 20-30 min | Fmoc-L-Leu-OH | 2.0 AA/2.2 DIC/2.0 Oxyma |
| | 20% Pip/DMF | | 4 hr, rt |
| 7 | 3 × 20-30 min | Fmoc-L-2-Me-Ile-OH | 2.0 AA/2.2 DIC/2.0 Oxyma |
| | 20% Pip/DMF | | 12-18 hr, rt |
| | | | capping procedure |
| 8 | 3 × 20-30 min | Fmoc-L-Ile-OH | 2.0 AA/2.2 DIC/2.0 Oxyma |
| | 20% Pip/DMF | | 12-18 hr, rt |
| | | | capping procedure |
| 9 | 2 × 20-30 min | Fmoc-L-Ser(t-Bu)-OH | 2.0 AA/2.2 DIC/2.0 Oxyma |
| | 20% Pip/DMF | | 4-6 hr, rt |
| 10 | 2 × 20-30 min | Fmoc-L-Tyr(t-Bu)-OH | 2.0 AA/2.2 DIC/2.0 Oxyma |
| | 20% Pip/DMF | | 4-6 hr, rt |
| 11 | 2 × 20-30 min | Fmoc-L-Asp(t-Bu)-OH | 2.0 AA/2.2 DIC/2.0 Oxyma |
| | 20% Pip/DMF | | 4-6 hr, rt |
| 12 | 2 × 10 min | Fmoc-L-Ser(t-Bu)-OH | 2.0 AA/2.2 DIC/2.0 Oxyma |
| | 5% Oxyma/20% | | 4-6 hr, rt |
| | Pip/DMF | | |
| 13 | 2 × 10 min | Fmoc-L-Thr(t-Bu)-OH | 2.0 AA/2.2 DIC/2.0 Oxyma |
| | 5% Oxyma/20% | | 4-6 hr, rt |
| | Pip/DMF | | |
| 14 | 2 × 10 min | Fmoc-L-Phe(t-Bu)-OH | 2.0 AA/2.2 DIC/2.0 Oxyma |
| | 5% Oxyma/20% | | 4-6 hr, rt |
| | Pip/DMF | | |
| 15 | 2 × 10 min | Fmoc-L-Thr(t-Bu)-OH | 2.0 AA/2.2 DIC/2.2 Oxyma |
| | 5% Oxyma/20% | | 4-6 hr, rt |
| | Pip/DMF | | |
| 16 | 2 × 10 min | Boc-L-Tyr(t-Bu)- | 1.5 AA/1.65 DIC/1.5 Oxyma |
| | 5% Oxyma/20% | Aib-L-Gln(Trt)- | 4-6 hr, rt |
| | Pip/DMF | Gly-OH | |

The fragment on resin is swelled with DCM (3×10 V) using filter reactor. Deprotection cocktail is prepared by mixing 10 V of TFA, 0.4 V of TIPS, 0.4 V H₂O and 0.3 Weight V of DTT and is stirred until homogeneous. Cocktail is added to the resin, and the resulting slurry is stirred for 3 hr at rt. The resin is filtered and is washed with DCM (2×3 V). Then, the resulting filtrates are combined and cooled to about −10° C., and 75 V of MTBE is added slowly. The resulting slurry is filtered and is washed with 2×10 V of MTBE. The solid is dried in a vacuum dryer (40° C.) to yield product as a white solid.

Crude peptide hydrazide is dissolved in 30 V of ligation buffer (6 M guanidine hydrochloride and 0.2 M sodium hydrogen phosphate monobasic buffer, pH 3.35) and cooled to about −15° C. 1 M sodium nitrite solution (5.0-10.0 equiv) is added to the hydrazide solution and is allowed to stir for 10 min at about −15° C. After 10 min, 2,2,2-trifluoroethanethiol (20.0 equiv, pH 7.0) is added to the peptidyl azide generated from the oxidation of the peptide hydrazide. The pH of the reaction mixture is adjusted to 7.0 with 5 N sodium hydroxide solution. Thiolysis of the peptidyl azide is allowed to run for 1 hr, and then the resulting peptide thioester is used directly in ligation chemistry or purified via reverse phase chromatography (see, Huang et al. (2014) Tetrahedron 70:2951-2955).

### Example 18: Solid Phase Peptide Synthesis of Intermediate Compound 18

Intermediate Compound 18 (SEQ ID NO:24), or a pharmaceutically acceptable salt thereof, can be synthesized by standard SPPS. Briefly, SPPS is conducted using Sieber resin (loading factor 0.6-0.9 mmol/g) with the conditions set forth below in Table 18.

TABLE 18
SPPS Conditions for Example 18.

| | | | SPPS Conditions |
|---|---|---|---|
| Cycle | Deprotection | Amino Acid | Solvent for Couplings: DMF |
| 1 | 2 × 30 min | Fmoc-L-Ser(t-Bu)-OH | 2.0 AA/2.2 DIC/2.0 Oxyma |
| | 20% Pip/DMF | | 4 hr, rt |
| 2 | 2 × 30 min | Fmoc-L-Pro-OH | 2.0 AA/2.2 DIC/2.0 Oxyma |
| | 20% Pip/DMF | | 6 hr, rt |
| 3 | 2 × 30 min | Fmoc-L-Pro-OH | 2.0 AA/2.2 DIC/2.0 Oxyma |
| | 20% Pip/DMF | | 6 hr, rt |
| 4 | 2 × 30 min | Fmoc-L-Pro-OH | 2.0 AA/2.2 DIC/2.0 Oxyma |
| | 20% Pip/DMF | | 6 hr, rt |
| 3-4* | Alternatively use coupling of Fmoc-L-Pro-Pro-OH dimer instead of step 3 & 4 | | |
| 5 | 2 × 30 min | Fmoc-L-Ala-OH | 2.0 AA/2.2 DIC/2.0 Oxyma |
| | 20% Pip/DMF | | 4 hr, rt |
| 6 | 2 × 30 min | Fmoc-Gly-OH | 2.0 AA/2.2 DIC/2.0 Oxyma |
| | 20% Pip/DMF | | 4 hr, rt |
| 7 | 2 × 30 min | Fmoc-L-Ser(t-Bu)-OH | 2.0 AA/2.2 DIC/2.0 Oxyma |
| | 20% Pip/DMF | | 4 hr, rt |
| 8 | 2 × 30 min | Fmoc-L-Ser(t-Bu)-OH | 2.0 AA/2.2 DIC/2.0 Oxyma |
| | 20% Pip/DMF | | 4 hr, rt |
| 9 | 2 × 30 min | Fmoc-L-Pro-OH | 2.0 AA/2.2 DIC/2.0 Oxyma |
| | 20% Pip/DMF | | 4 hr, rt |
| 10 | 2 × 30 min | Fmoc-Gly-OH | 2.0 AA/2.2 DIC/2.0 Oxyma |
| | 20% Pip/DMF | | 4 hr, rt |
| 11 | 2 × 30 min | Fmoc-Gly-OH | 2.0 AA/2.2 DIC/2.0 Oxyma |
| | 20% Pip/DMF | | 4 hr, rt |
| 10-11* | Alternatively use coupling of Fmoc-Gly-Gly-OH dimer instead of step 3 & 4 | | |
| 12 | 2 × 20-30 min | Fmoc-L-Glu(t-Bu)-OH | 2.5 AA/2.5 PyBOP/5.0 DIPEA |
| | 20% Pip/DMF | | 4 hr, rt |
| 13 | 2 × 20-30 min | Fmoc-L-Leu-OH | 2.0 AA/2.2 DIC/2.0 Oxyma |
| | 20% Pip/DMF | | 4-8 hr, rt |
| 14 | 2 × 20-30 min | Fmoc-L-Leu-OH | 2.0 AA/2.2 DIC/2.0 Oxyma |
| | 20% Pip/DMF | | 4-8 hr, rt |
| 15 | 2 × 20-30 min | Fmoc-L-Tyr(t-Bu)-OH | 2.0 AA/2.2 DIC/2.0 Oxyma |
| | 20% Pip/DMF | | 4-8 hr, rt |
| 16 | 2 × 20-30 min | Fmoc-L-Glu(t-Bu)-OH | 2.0 AA/2.2 DIC/2.0 Oxyma |
| | 20% Pip/DMF | | 4-8 hr, rt |
| 17 | 2 × 20-30 min | Fmoc-L-Ile-OH | 2.0 AA/2.2 DIC/2.0 Oxyma |
| | 20% Pip/DMF | | 4-8 hr, rt |
| 18 | 2 × 20-30 min | Fmoc-L-Phe-OH | 2.0 AA/2.2 DIC/2.0 Oxyma |
| | 20% Pip/DMF | | 4-8 hr, rt |
| 19 | 2 × 20-30 min | Fmoc-L-Cys(Trt)-OH | 2.0 AA/2.2 DIC/2.0 Oxyma |
| | 20% Pip/DMF | | 4-8 hr, rt |

Cleavage and Deprotection: The fragment on resin is swelled with DCM (3×10 V) using filter reactor. Deprotection cocktail is prepared by mixing 10 V of TFA, 0.4 V of TIPS, 0.4 V H₂O and 0.3 Weight V of DTT and is stirred until homogeneous. Cocktail is added to the resin, and the resulting slurry is stirred for 3 hr at rt. The resin is filtered and is washed with DCM (2×3 V). Then, the resulting filtrates are combined and cooled to about −10° C., and 75 V of MTBE is added slowly. The resulting slurry is filtered and is washed with 2×10 V of MTBE. The solid is dried in a vacuum dryer (40° C.) to yield product as a white solid.

### Example 19: Hybrid Liquid Solid Phase Peptide Synthesis of Intermediate Compound 19

Intermediate Compound 19 (SEQ ID NO:25), or a pharmaceutically acceptable salt thereof, can be synthesized by standard SPPS. Briefly, SPPS is conducted using Fmoc-L-Lys(t-BuOOC-(CH₂)₁₈-COO-γ-L-Glu-AEEA)-Lys-2-CTC-hydrazine resin (loading factor 0.6-0.9 mmol/g) with the conditions set forth below in Table 19.

TABLE 19
SPPS Conditions for Example 19.

| | | | SPPS Conditions |
|---|---|---|---|
| Cycle | Deprotection | Amino Acid | Solvent for Couplings: DMF |
| 1 | 2 × 20-30 min | Fmoc-L-Lys(Boc)-OH | 2.0 AA/2.2 DIC/2.0 Oxyma |
| | 20% Pip/DMF | | 4 hr, rt |
| 2 | 2 × 20-30 min | Fmoc-L-Asp(t-Bu)-OH | 2.0 AA/2.2 DIC/2.0 Oxyma |
| | 20% Pip/DMF | | 4 hr, rt |
| 3 | 2 × 20-30 min | Fmoc-L-Leu-OH | 2.0 AA/2.2 DIC/2.0 Oxyma |
| | 20% Pip/DMF | | 4 hr, rt |
| 4 | 3 × 20-30 min | Fmoc-L-2-Me-Ile-OH | 2.0 AA/2.2 DIC/2.0 Oxyma |
| | 20% Pip/DMF | | 12-18 hr, rt |
| | | | capping procedure |
| 5 | 3 × 20-30 min | Fmoc-L-Ile-OH | 2.0 AA/2.2 DIC/2.0 Oxyma |
| | 20% Pip/DMF | | 12-18 hr, rt |
| | | | capping procedure |
| 6 | 2 × 20-30 min | Fmoc-L-Ser(t-Bu)-OH | 2.0 AA/2.2 DIC/2.0 Oxyma |
| | 20% Pip/DMF | | 4-6 hr, rt |
| 7 | 2 × 20-30 min | Fmoc-L-Tyr(t-Bu)-OH | 2.0 AA/2.2 DIC/2.0 Oxyma |
| | 20% Pip/DMF | | 4-6 hr, rt |
| 8 | 2 × 20-30 min | Fmoc-L-Asp(t-Bu)-OH | 2.0 AA/2.2 DIC/2.0 Oxyma |
| | 20% Pip/DMF | | 4-6 hr, rt |
| 9 | 2 × 10 min | Fmoc-L-Ser(t-Bu)-OH | 2.0 AA/2.2 DIC/2.0 Oxyma |
| | 5% Oxyma/20% | | 4-6 hr, rt |
| | Pip/DMF | | |
| 10 | 2 × 10 min | Fmoc-L-Thr(t-Bu)-OH | 2.0 AA/2.2 DIC/2.0 Oxyma |
| | 5% Oxyma/20% | | 4-6 hr, rt |
| | Pip/DMF | | |
| 11 | 2 × 10 min | Fmoc-L-Phe(t-Bu)-OH | 2.0 AA/2.2 DIC/2.0 Oxyma |
| | 5% Oxyma/20% | | 4-6 hr, rt |
| | Pip/DMF | | |
| 12 | 2 × 10 min | Fmoc-L-Thr(t-Bu)-OH | 2.0 AA/2.2 DIC/2.2 Oxyma |
| | 5% Oxyma/20% | | 4-6 hr, rt |
| | Pip/DMF | | |
| 13 | 2 × 10 min | Boc-L-Tyr(t-Bu)- | 1.5 AA/1.65 DIC/1.5 Oxyma |
| | 5% Oxyma/20% | Aib-L-Gln(Trt)- | 4-6 hr, rt |
| | Pip/DMF | Gly-OH | |

The fragment on resin is swelled with DCM (3×10 V) using filter reactor. Deprotection cocktail is prepared by mixing 10 V of TFA, 0.4 V of TIPS, 0.4 V H₂O and 0.3 Weight V of DTT and is stirred until homogeneous. Cocktail is added to the resin, and the resulting slurry is stirred for 3 hr at rt. The resin is filtered and is washed with DCM (2×3 V). Then, the resulting filtrates are combined and cooled to about −10° C., and 75 V of MTBE is added slowly. The resulting slurry is filtered and is washed with 2×10 V of MTBE. The solid is dried in a vacuum dryer (40° C.) to yield product as a white solid.

Crude peptide hydrazide is dissolved in 30 V of the ligation buffer (6 M guanidine hydrochloride and 0.2 M sodium hydrogen phosphate monobasic buffer, pH 3.35) and cooled to about −15° C. 1 M sodium nitrite solution (5.0-10.0 equiv) is added to the hydrazide solution and is allowed to stir for 10 min at about −15° C. After 10 min, 2,2,2-trifluoroethanethiol (20.0 equiv, pH 7.0) is added to the peptidyl azide generated from oxidizing the peptide hydrazide. The pH of the reaction mixture is adjusted to 7.0 with 5 N sodium hydroxide solution. Thiolysis of the peptidyl azide is allowed to run for 1 hr, and then the resulting peptide thioester is used directly in ligation chemistry or purified via reverse phase chromatography (see, Huang (2014)).

### Example 20: Solid Phase Peptide Synthesis of Intermediate Compound 20

Intermediate Compound 20 (SEQ ID NO:26), or a pharmaceutically acceptable salt thereof, can be synthesized by standard SPPS. Briefly, SPPS is conducted using Sieber resin (loading factor 0.6-0.9 mmol/g) with the conditions set forth below in Table 20.

TABLE 20
SPPS Conditions for Example 20.

| | | | SPPS Conditions |
|---|---|---|---|
| Cycle | Deprotection | Amino Acid | Solvent for Couplings: DMF |
| 1 | 2 × 30 min | Fmoc-L-Ser(t-Bu)-OH | 2.0 AA/2.2 DIC/2.0 Oxyma |
| | 20% Pip/DMF | | 4 hr, rt |
| 2 | 2 × 30 min | Fmoc-L-Pro-OH | 2.0 AA/2.2 DIC/2.0 Oxyma |
| | 20% Pip/DMF | | 6 hr, rt |
| 3 | 2 × 30 min | Fmoc-L-Pro-OH | 2.0 AA/2.2 DIC/2.0 Oxyma |
| | 20% Pip/DMF | | 6 hr, rt |
| 4 | 2 × 30 min | Fmoc-L-Pro-OH | 2.0 AA/2.2 DIC/2.0 Oxyma |
| | 20% Pip/DMF | | 6 hr, rt |
| 3-4* | Alternatively use coupling of Fmoc-L-Pro-Pro-OH dimer instead of step 3 & 4 | | |
| 5 | 2 × 30 min | Fmoc-L-Ala-OH | 2.0 AA/2.2 DIC/2.0 Oxyma |
| | 20% Pip/DMF | | 4 hr, rt |
| 6 | 2 × 30 min | Fmoc-Gly-OH | 2.0 AA/2.2 DIC/2.0 Oxyma |
| | 20% Pip/DMF | | 4 hr, rt |
| 7 | 2 × 30 min | Fmoc-L-Ser(t-Bu)-OH | 2.0 AA/2.2 DIC/2.0 Oxyma |
| | 20% Pip/DMF | | 4 hr, rt |
| 8 | 2 × 30 min | Fmoc-L-Ser(t-Bu)-OH | 2.0 AA/2.2 DIC/2.0 Oxyma |
| | 20% Pip/DMF | | 4 hr, rt |
| 9 | 2 × 30 min | Fmoc-L-Pro-OH | 2.0 AA/2.2 DIC/2.0 Oxyma |
| | 20% Pip/DMF | | 4 hr, rt |
| 10 | 2 × 30 min | Fmoc-Gly-OH | 2.0 AA/2.2 DIC/2.0 Oxyma |
| | 20% Pip/DMF | | 4 hr, rt |
| 11 | 2 × 30 min | Fmoc-Gly-OH | 2.0 AA/2.2 DIC/2.0 Oxyma |
| | 20% Pip/DMF | | 4 hr, rt |
| 10-11* | Alternatively use coupling of Fmoc-Gly-Gly-OH dimer instead of step 3 & 4 | | |
| 12 | 2 × 20-30 min | Fmoc-L-Glu(t-Bu)-OH | 2.5 AA/2.5 PyBOP/5.0 DIPEA |
| | 20% Pip/DMF | | 4 hr, rt |
| 13 | 2 × 20-30 min | Fmoc-L-Leu-OH | 2.0 AA/2.2 DIC/2.0 Oxyma |
| | 20% Pip/DMF | | 4-8 hr, rt |
| 14 | 2 × 20-30 min | Fmoc-L-Leu-OH | 2.0 AA/2.2 DIC/2.0 Oxyma |
| | 20% Pip/DMF | | 4-8 hr, rt |
| 15 | 2 × 20-30 min | Fmoc-L-Tyr(t-Bu)-OH | 2.0 AA/2.2 DIC/2.0 Oxyma |
| | 20% Pip/DMF | | 4-8 hr, rt |
| 16 | 2 × 20-30 min | Fmoc-L-Glu(t-Bu)-OH | 2.0 AA/2.2 DIC/2.0 Oxyma |
| | 20% Pip/DMF | | 4-8 hr, rt |
| 17 | 2 × 20-30 min | Fmoc-L-Ile-OH | 2.0 AA/2.2 DIC/2.0 Oxyma |
| | 20% Pip/DMF | | 4-8 hr, rt |
| 18 | 2 × 20-30 min | Fmoc-L-Phe-OH | 2.0 AA/2.2 DIC/2.0 Oxyma |
| | 20% Pip/DMF | | 4-8 hr, rt |
| 19 | 2 × 20-30 min | Fmoc-L-Ala-OH | 2.0 AA/2.2 DIC/2.0 Oxyma |
| | 20% Pip/DMF | | 4-8 hr, rt |
| 20 | 3 × 20-30 min | Fmoc-Aib-OH | 2.0 AA/2.2 DIC/2.0 Oxyma |
| | 20% Pip/DMF | | 8-12 hr, rt |
| | | | capping procedure |
| 21 | 3 × 20-30 min | Fmoc-L-Gln(Trt)-OH | 2.0 AA/2.2 DIC/2.0 Oxyma |
| | 20% Pip/DMF | | 12-18 hr, rt |
| | | | capping procedure |
| 22 | 2 × 20-30 min | Fmoc-L-Cys(Trt)-OH | AA/2.2 DIC/2.0 Oxyma |
| | 20% Pip/DMF | | 4-8 hr, rt |

Cleavage and Deprotection: The fragment on resin is swelled with DCM (3×10 V) using filter reactor. Deprotection cocktail is prepared by mixing 10 V of TFA, 0.4 V of TIPS, 0.4 V H₂O and 0.3 Weight V of DTT and is stirred until homogeneous. Cocktail is added to the resin, and the resulting slurry is stirred for 3 hr at rt. The resin is filtered and is washed with DCM (2×3 V). Then, the resulting filtrates are combined and cooled to about −10° C., and 75 V of MTBE is added slowly. The resulting slurry is filtered and is washed with 2×10 V of MTBE. The solid is dried in a vacuum dryer (40° C.) to yield product as a white solid.

### Example 21: Hybrid Liquid Solid Phase Peptide Synthesis of Intermediate Compound 21

Intermediate Compound 21 (SEQ ID NO:27), or a pharmaceutically acceptable salt thereof, can be synthesized by standard SPPS. Briefly, SPPS is conducted using Fmoc-Lys(Boc)-2-CTC-hydrazine resin (loading factor 0.6-0.9 mmol/g) with the conditions set forth below in Table 21.

TABLE 21
SPPS Conditions for Example 21.

| | | | SPPS Conditions |
|---|---|---|---|
| Cycle | Deprotection | Amino Acid | Solvent for Couplings: DMF |
| 1 | 2 × 20-30 min | Fmoc-L-Lys(PhAc)-OH | 2.0 AA/2.2 DIC/2.0 Oxyma |
| | 20% Pip/DMF | | 4 hr, rt |
| 2 | 2 × 20-30 min | Fmoc-L-Asp(t-Bu)-OH | 2.0 AA/2.2 DIC/2.0 Oxyma |
| | 20% Pip/DMF | | 4 hr, rt |
| 3 | 2 × 20-30 min | Fmoc-L-Leu-OH | 2.0 AA/2.2 DIC/2.0 Oxyma |
| | 20% Pip/DMF | | 4 hr, rt |
| 4 | 3 × 20-30 min | Fmoc-L-2-Me-Ile-OH | 2.0 AA/2.2 DIC/2.0 Oxyma |
| | 20% Pip/DMF | | 12-18 hr, rt |
| | | | capping procedure |
| 5 | 3 × 20-30 min | Fmoc-L-Ile-OH | 2.0 AA/2.2 DIC/2.0 Oxyma |
| | 20% Pip/DMF | | 12-18 hr, rt |
| | | | capping procedure |
| 6 | 2 × 20-30 min | Fmoc-L-Ser(t-Bu)-OH | 2.0 AA/2.2 DIC/2.0 Oxyma |
| | 20% Pip/DMF | | 4-6 hr, rt |
| 7 | 2 × 20-30 min | Fmoc-L-Tyr(t-Bu)-OH | 2.0 AA/2.2 DIC/2.0 Oxyma |
| | 20% Pip/DMF | | 4-6 hr, rt |
| 8 | 2 × 20-30 min | Fmoc-L-Asp(t-Bu)-OH | 2.0 AA/2.2 DIC/2.0 Oxyma |
| | 20% Pip/DMF | | 4-6 hr, rt |
| 9 | 2 × 10 min | Fmoc-L-Ser(t-Bu)-OH | 2.0 AA/2.2 DIC/2.0 Oxyma |
| | 5% Oxyma/20% | | 4-6 hr, rt |
| | Pip/DMF | | |
| 10 | 2 × 10 min | Fmoc-L-Thr(t-Bu)-OH | 2.0 AA/2.2 DIC/2.0 Oxyma, |
| | 5% Oxyma/20% | | 4-6 h, rt. |
| | Pip/DMF | | |
| 11 | 2 × 10 min | Fmoc-L-Phe(t-Bu)-OH | 2.0 AA/2.2 DIC/2.0 Oxyma |
| | 5% Oxyma/20% | | 4-6 hr, rt |
| | Pip/DMF | | |
| 12 | 2 × 10 min | Fmoc-L-Thr(t-Bu)-OH | 2.0 AA/2.2 DIC/2.2 Oxyma |
| | 5% Oxyma/20% | | 4-6 hr, rt |
| | Pip/DMF | | |
| 13 | 2 × 10 min | Boc-L-Tyr(t-Bu)- | 1.5 AA/1.65 DIC/1.5 Oxyma |
| | 5% Oxyma/20% | Aib-L-Gln(Trt)- | 4-6 hr, rt |
| | Pip/DMF | Gly-OH | |

The fragment on resin is swelled with DCM (3×10 V) using filter reactor. Deprotection cocktail is prepared by mixing 10 V of TFA, 0.4 V of TIPS, 0.4 V H₂O and 0.3 Weight V of DTT and is stirred until homogeneous. Cocktail is added to the resin, and the resulting slurry is stirred for 3 hr at rt. The resin is filtered and is washed with DCM (2×3 V). Then, the resulting filtrates are combined and cooled to about −10° C., and 75 V of MTBE is added slowly. The resulting slurry is filtered and is washed with 2×10 V of MTBE. The solid is dried in a vacuum dryer (40° C.) to yield product as a white solid.

Crude peptide hydrazide is dissolved in 30 V of the ligation buffer (6 M guanidine hydrochloride and 0.2 M sodium hydrogen phosphate monobasic buffer, pH 3.35) and cooled to about −15° C. 1 M sodium nitrite solution (5.0-10.0 equiv) is added to the hydrazide solution and is allowed to stir for 10 min at about −15° C. After 10 min, 2,2,2-trifluoroethanethiol (20.0 equiv, pH 7.0) is added to the peptidyl azide generated by oxidizing the peptide hydrazide. The pH of the reaction mixture is adjusted to 7.0 with 5 N sodium hydroxide solution. Thiolysis of the peptidyl azide runs for 1 hr, and then the resulting peptide thioester is used directly in ligation chemistry or purified via reverse phase chromatography (see, Huang (2014)).

### Example 22: Solid Phase Peptide Synthesis of Intermediate Compound 22

Intermediate Compound 22 (SEQ ID NO:28), or a pharmaceutically acceptable salt thereof, can be synthesized by standard SPPS. Briefly, SPPS is conducted using Fmoc-Gly-2-CTC resin (loading factor 0.6-0.9 mmol/g) with the conditions set forth below in Table 22.

TABLE 22
SPPS Conditions for Example 22.

| | | | SPPS Conditions |
|---|---|---|---|
| Cycle | Deprotection | Amino Acid | Solvent for Couplings: DMF |
| 1 | 2 × 30 min | Fmoc-L-Gln(Trt)-OH | 2.0 AA/2.2 DIC/2.0 Oxyma |
| | 20% Pip/DMF | | 6-9 hr, rt |
| 2 | 2 × 30 min | Fmoc-L-Aib-OH | 2.0 AA/2.2 DIC/2.0 Oxyma |
| | 20% Pip/DMF | | 4-8 hr, rt |
| 3 | 2 × 30 min | Boc-L-Tyr(Boc)-OH | 2.0 AA/2.2 DIC/2.0 Oxyma |
| | 20% Pip/DMF | | 12-18 hr, rt |

Cleavage and Deprotection: Tetramer on CTC resin is swelled using DCM (5-10 V) for 2×30 min. 3.5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% TFA/DCM followed by filtrate neutralization is repeated four more times. The resin is washed with 3.5 V of DCM and is stirred for 5-10 min. All the filtrates and washes are combined. The fragment solution is concentrated under vacuum to 1.5 V maintaining temperature at ≤35° C. 5 V of IPAc is added to the solution, and residual IPAc/DCM solvents are removed under vacuum to 1.5 V maintaining temperature at ≤40° C. Addition of 5 V of IPAc and distillation under vacuum are repeated to produce 3.5 V of the final fragment solution. This solution is then washed with 3×2 V of 5.0% NaCl solution, and then IPAc is removed to 1.5 V under reduced pressure maintaining temperature at ≤40° C. 4-5 V heptane is added to the solution at 40° C. Then, temperature is reduced to about 15° C., and the resulting slurry is stirred for 30 min. The IPAc/heptane solvents are removed to 3.5 V under reduced pressure maintaining temperature at ≤40° C. The heptane charge and distillation are repeated, and the resulting slurry of precipitated fragment is cooled to about 20° C., is filtered, is washed with 2 V of heptane, and the resulting solid is dried at about 35° C.

### Example 23: Solid Phase Peptide Synthesis of Intermediate Compound 23

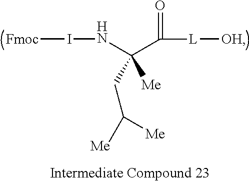

or a pharmaceutically acceptable salt thereof, can be synthesized by standard SPPS. Briefly, SPPS is conducted using Fmoc-Leu-2-CTC resin (loading factor 0.6-0.9 mmol/g) with the conditions set forth below in Table 23.

TABLE 23
SPPS Conditions for Example 23.

| | | | SPPS Conditions |
|---|---|---|---|
| Cycle | Deprotection | Amino Acid | Solvent for Couplings: DMF |
| 1 | 2 × 30 min | Fmoc-L-2-Me-Leu-OH | 2.0 AA/2.2 DIC/2.0 Oxyma |
| | 20% Pip/DMF | | 6-9 hr, rt |
| 2 | 2 × 30 min | Fmoc-L-Ile-OH | 2.0 AA/2.2 DIC/2.0 Oxyma |
| | 20% Pip/DMF | | 16-18 hr, rt |

Cleavage and Deprotection: Tetramer on CTC resin is swelled using DCM (5-10 V) for 2×30 min. 3.5 V of 1% TFA/DCM is charged to the reactor, and the resulting suspension of the resin is stirred for 10-15 min under nitrogen maintaining temperature at about 25° C. The filtrate is removed and immediately neutralized by slow addition of a 1.05 equivalent amount of pyridine. Resin treatment with 1% TFA/DCM followed by filtrate neutralization is repeated four more times. The resin is washed with 3.5 V of DCM and is stirred for 5-10 min. All the filtrates and washes are combined. The fragment solution is concentrated under vacuum to 1.5 V maintaining temperature at ≤35° C. 5 V of IPAc is added to the solution, and residual IPAc/DCM solvents are removed under vacuum to 1.5 V maintaining temperature at ≤40° C. Addition of 5 V of IPAc and distillation under vacuum are repeated to produce 3.5 V of the final fragment solution. This solution is then washed with 3×2 V of 5.0% NaCl solution, and IPAc is removed to 1.5 V under reduced pressure maintaining temperature at ≤40° C. 4-5 V heptane is added to the solution at about 40° C. Then, temperature is reduced to about 15° C., and the resulting slurry is stirred for 30 min. The IPAc/heptane solvents are removed to 3.5 V under reduced pressure maintaining temperature at ≤40° C. The heptane charge and distillation are repeated, the resulting slurry of precipitated fragment is cooled to about 20° C., is filtered and washed with 2 V of heptane, and the resulting solid is dried at about 35° C.

### Example 24: Liquid Phase Peptide Synthesis of Intermediate Compound 24

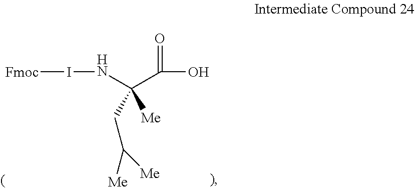

or a pharmaceutically acceptable salt thereof, can be synthesized by coupling of H-L-2-Me-Leu and Fmoc-L-Ile-OH using standard coupling chemistry in solution followed by work up and isolation.

### Example 25: Solid Phase Peptide Synthesis of Fatty Acid Moiety (Compound 25)

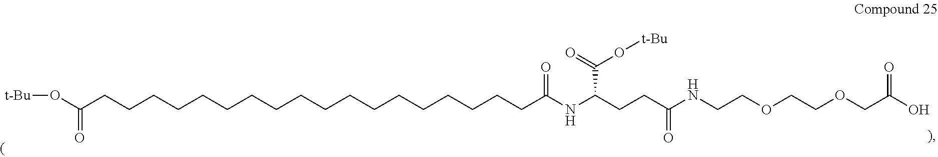 [SMILES: CC(C)(C)OC(=O)CCCCCCCCCCCCCCCCCCC(=O)N[C@@H](CCC(=O)NCCOCCOCC(=O)O)C(=O)OC(C)(C)C]

or a pharmaceutically acceptable salt thereof, can be synthesized by standard SPPS. Briefly, SPPS is conducted using Fmoc-PEG-2-CTC resin (loading factor 0.6-1.1 mmol/g) with the conditions set forth below in Table 24.

TABLE 24
SPPS Conditions for Example 25.

| | | | SPPS Conditions |
|---|---|---|---|
| Cycle | Deprotection | Amino Acid | Solvent for Couplings: DMF |
| 1 | 2 × 20-30 min | Fmoc-γ-Glu-Ot-Bu | 2.0 AA/2.2 DIC/2.0 Oxyma |
| | 20% Pip/DMF | | 4 hr, rt |
| 2 | 2 × 20-30 min | t-BuOOC—(CH₂)₁₈—COO | 2.0 AA/2.2 DIC/2.0 Oxyma |
| | 20% Pip/DMF | | 4 hr, rt |

### Example 26: Hybrid Liquid Solid Phase Synthesis of Incretin Analog from Four Intermediate Compounds Via Chemical Conjugation

Coupling Protocol: The incretin analog of SEQ ID NO:6 can be made by coupling SEQ ID NOS:7, 8, 9 and 10 via HLSPS. Briefly, a solution of SEQ ID NO:7 (1.05-1.30 mmol) and a solution of SEQ ID NO:8 (1.00 mmol) in 30-40 V of DMSO/ACN (70:30) are coupled using PyBOP, HATU or PyOXim reagent (1.30-2.00 mmol) and DIEA (4.00-5.00 mmol) at rt. The mixture is stirred at rt for 2-4 hr. Then, 10 equivalents of DEA are added, and the mixture is stirred for 4 hr. The mixture is quenched with 20 V of 15-20% brine solution, then an additional 10 V of water is added and is stirred for 10 min. The resulting slurry is filtered, and the solid is washed with 3×10 V of water. The solid is dried in a vacuum dryer (40° C.) to yield product as a white solid.

Next, a solution of the coupled SEQ ID NOS:7+8 (1.00 mmol) and a solution of SEQ ID NO:9 (1.05-1.30 mmol) in 30-40 V of DMSO/ACN (70:30) are coupled using PyBOP, HATU or PyOXim reagent (1.30-2.00 mmol) and DIEA (4.00-5.00 mmol) at rt. The mixture is stirred at rt for 2-4 hr. Then, 10 equivalents of DEA are added, and the mixture is stirred for 2-4 hr. The mixture is quenched with 20 V of 15-20% brine solution, then an additional 10 V of water is added and is stirred for 10 min. The resulting slurry is filtered, and the solid is washed with 3×10 V of water. The solid is dried in a vacuum dryer (40° C.) to yield product as a white solid.

Next, a solution of the coupled SEQ ID NOS:7+8+9 (1.00 mmol) and a solution of SEQ ID NO:10 (1.20-1.30 mmol) in 30-40 V of DMSO/ACN (70:30) are coupled using PyBOP, HATU or PyOXim reagent (1.50-2.00 mmol) and DIEA (4.00-5.00 mmol) at rt. The mixture is stirred at rt for 3-4 hr. The mixture is quenched with 20 V of 15-20% brine solution, and then an additional 10 V of water is added and is stirred for 10 min. The resulting slurry is filtered, and the solid is washed with 3×10 V of water. The solid is dried in a vacuum dryer (40° C.) to yield product as a white solid.

Global Deprotection: Deprotection cocktail is prepared by mixing 10 V of TFA, 2 V of DCM, 0.4 V of TIPS, 0.4 V H₂O and 0.3 weight V of DTT and is stirred until homogeneous. The cocktail is cooled to about 15° C., and then solid, coupled SEQ ID NOS:7+8+9+10 is added, and the resulting reaction mixture is warmed to rt and is stirred for 3 hr at rt. The mixture is cooled to about −10° C., and 75 V of MTBE is added slowly. The resulting slurry is filtered and is washed with 2×10 V of MTBE. The solid is dried in a vacuum dryer (40° C.) to yield product SEQ ID NO:6 as a white solid.

### Example 27: Hybrid Liquid Solid Phase Synthesis of Incretin Analog from Four Intermediate Compounds Via Chemical Conjugation

Here, the incretin analog of SEQ ID NO:6 is made by coupling SEQ ID NOS:7, 11, 12 and 10 via convergent solid-phase peptide synthesis (CSPPS) essentially as described in Example 26 for coupling SEQ ID NOS:7, 8, 9 and 10.

### Example 28: Hybrid Liquid Solid Phase Synthesis of Incretin Analog from Four Intermediate Fragments Via Chemical Conjugation

Here, the incretin analog of SEQ ID NO:6 is made by coupling SEQ ID NOS:7, 13, 14 and 10 via CSPPS essentially as described in Example 26 for coupling SEQ ID NOS:7, 8, 9 and 10.

### Example 29: Hybrid Liquid Solid Phase Synthesis of Incretin Analog from Three Intermediate Fragments Via Chemical Conjugation

The incretin analog of SEQ ID NO:6 can be made by coupling SEQ ID NOS:7, 13 and 15 via CSPPS. Briefly, a solution of SEQ ID NO:7 (1.05-1.30 mmol) and a solution of SEQ ID NO:13 (1.00 mmol) in 30-40 V of DMSO/ACN (70:30) are coupled using PyBOP, HATU or PyOXim reagent (1.30-2.00 mmol) and DIEA (4.00-5.00 mmol) at rt. The mixture is stirred at rt for 2-4 hr. Then, 10 equivalents of DEA are added, and the mixture is stirred for 4 hr. The mixture is quenched with 20 V of 15-20% brine solution, and then an additional 10 V of water is added and is stirred for 10 min. The resulting slurry is filtered, and the solid is washed with 3×10 V of water. The solid is dried in a vacuum dryer (40° C.) to yield product as a white solid.

Next, a solution of the coupled SEQ ID NOS:7+13 (1.00 mmol) and a solution of SEQ ID NO:15 (1.20-1.30 mmol) in 30-40 V of DMSO/ACN (70:30) are coupled using PyBOP, HATU or PyOXim reagent (1.50-2.00 mmol) and DIEA (4.00-5.00 mmol) at rt. The mixture is stirred at rt for 2-4 hr. Then, 10 equivalents of DEA are added, and the mixture is stirred for 2-4 hr. The mixture is quenched with 20 V of 15-20% brine solution, and then an additional 10 V of water is added and stirred for 10 min. The resulting slurry is filtered, and the solid is washed with 3×10 V of water. The solid is dried in a vacuum dryer (40° C.) to yield product as a white solid.

Global Deprotection: Deprotection cocktail is prepared by mixing 10 V of TFA, 2 V of DCM, 0.4 V of TIPS, 0.4 V H₂O and 0.3 weight V of DTT and is stirred until homogeneous. The cocktail is cooled to about 15° C., and then solid, coupled SEQ ID NOS:7+13+15 is added, and the resulting reaction mixture is warmed to rt and is stirred for 3 hr at rt. The mixture is cooled to about −10° C., and 75 V of MTBE added slowly. The resulting slurry is filtered and is washed with 2×10 V of MTBE. The solid is dried in a vacuum dryer (40° C.) to yield product SEQ 6 as a white solid.

### Example 30: Hybrid Liquid Solid Phase Synthesis of Incretin Analog from Three Intermediate Fragments Via Chemical Conjugation

The incretin analog of SEQ ID NO:6 can be made by coupling SEQ ID NOS:16, 9 and 10 via CSPPS. Briefly, a solution of SEQ ID NOS:16 (1.00 mmol) and a solution of SEQ ID NO:9 (1.05-1.30 mmol) in 30-40 V of DMSO/ACN (70:30) are coupled using PyBOP, HATU or PyOXim reagent (1.30-2.00 mmol) and DIEA (4.00-5.00 mmol) at rt. The mixture is stirred at rt for 3-4 hr. The mixture is quenched with 20 V of 15-20% brine solution, and then an additional 10 V of water is added and is stirred for 10 min. The resulting slurry is filtered, and the solid is washed with 3×10 V of water. The solid is dried in a vacuum dryer (40° C.) to yield product as a white solid.

Next, a solution of the coupled SEQ ID NOS:16+9 (1.00 mmol) and a solution of SEQ ID NO:10 (1.20-1.30 mmol) in 30-40 V of DMSO/ACN (70:30) are coupled using PyBOP, HATU or PyOXim reagent (1.50-2.00 mmol) and DIEA (4.00-5.00 mmol) at rt. The mixture is stirred at rt for 2-4 hr. Then, 10 equivalents of DEA are added, and the mixture is stirred for 2-4 hr. The mixture is quenched with 20 V of 15-20% brine solution, and then an additional 10 V of water is added and is stirred for 10 min. The resulting slurry is filtered, and the solid is washed with 3×10 V of water. The solid is dried in a vacuum dryer (40° C.) to yield product as a white solid.

Global Deprotection: Deprotection cocktail is prepared by mixing 10 V of TFA, 2 V of DCM, 0.4 V of TIPS, 0.4 V H₂O and 0.3 weight V of DTT and is stirred until homogeneous. The cocktail is cooled to about 15° C., and then solid, coupled SEQ ID NOS:16+9+10 is added, and the resulting reaction mixture is warmed to rt and is stirred for 3 hr at rt. The mixture is cooled to about −10° C., and 75 V of MTBE added slowly. The resulting slurry is filtered and is washed with 2×10 V of MTBE. The solid is dried in a vacuum dryer (40° C.) to yield product SEQ 6 as a white solid.

### Example 31: Hybrid Liquid Solid Phase Synthesis of Incretin Analog from Three Intermediate Fragments Via Chemical Conjugation

Here, the incretin analog of SEQ ID NO:6 is made by coupling SEQ ID NOS:18, 12 and 10 via CSPPS essentially as described in Example 30 for coupling SEQ ID NOS:16, 9 and 10.

### Example 32: Hybrid Liquid Solid Phase Synthesis of Incretin Analog from Two Intermediate Fragments Via Chemical Conjugation

The incretin analog of SEQ ID NO:6 can be made by coupling SEQ ID NOS:19 and 15 via CSPPS. Briefly, a solution of SEQ ID NOS:18 (1.00 mmol) and a solution of SEQ ID NO:15 (1.20-1.30 mmol) in 30-40 V of DMSO/ACN (70:30) are coupled using PyBOP, HATU or PyOXim reagent (1.50-2.00 mmol) and DIEA (4.00-5.00 mmol) at rt. The mixture is stirred at rt for 2-4 hr. The mixture is quenched with 20 V of 15-20% brine solution, and then an additional 10 V of water is added and is stirred for 10 min. The resulting slurry is filtered, and the solid is washed with 3×10 V of water. The solid is dried in a vacuum dryer (40° C.) to yield product as a white solid.

Global Deprotection: Deprotection cocktail is prepared by mixing 10 V of TFA, 2 V of DCM, 0.4 V of TIPS, 0.4 V H₂O and 0.3 weight V of DTT and is stirred until homogeneous. The cocktail is cooled to about 15° C., and then solid, coupled SEQ ID NOS:19+15 is added, and the resulting reaction mixture is warmed to rt and is stirred for 3 hr at rt. The mixture is cooled to about −10° C., and 75 V of MTBE added slowly. The resulting slurry is filtered and is washed with 2×10 V of MTBE. The solid is dried in a vacuum dryer (40° C.) to yield product SEQ ID NO:6 as a white solid.

### Example 33: Hybrid Liquid Solid Phase Synthesis of Incretin Analog from Two Intermediate Fragments Via Chemical Conjugation

Here, the incretin analog of SEQ ID NO:6 is made by coupling SEQ ID NOS:18 and 20 via CSPPS essentially as described in Example 32 for coupling of SEQ ID NOS:15 and 19.

### Example 34: Hybrid Liquid Solid Phase Synthesis of Incretin Analog from Two Intermediate Fragments Via Chemical Conjugation

Here, the incretin analog of SEQ ID NO:6 is made by coupling SEQ ID NOS:21 and 18 or SEQ ID NOS:22 and 19 via CSPPS essentially as described in Example 32 for coupling of SEQ ID NOS:15 and 19 with one exception that after the coupling of the two fragments, the protecting group on Lys17 is selectively removed via chemical transformation (conditions depend on the nature of the group) and then selectively acylated with the fatty acid side chain followed by global deprotection.

### Example 35: Hybrid Liquid Solid Phase Synthesis of Incretin Analog from Two Intermediate Fragments Via Native Chemical Ligation

The incretin analog of SEQ ID NO:6 can be made by coupling SEQ ID NOS:23 and 24 via native chemical ligation. Briefly, a peptide thioester of SEQ ID NOS:23 is dissolved in 30-50 V of ligation buffer (6 M guanidine hydrochloride and 0.2 M sodium hydrogen phosphate monobasic buffer, pH 7.04). N-terminal cysteine-containing peptide fragment SEQ ID NOS:24 (0.9-0.95 equiv) is added to the thioester solution. 40 equiv of 2,2,2-trifluoroethanethiol (pH 7.16) and 20 equiv of tris(2-carboxyethyl)phosphine (pH 7.0) are added to the reaction mixture, and the pH is adjusted to 7.0 with 5 N sodium hydroxide solution. The reaction is allowed to stir at room temperature for 24 hours, and the resulting solution is used directly in reverse phase purification.

### Example 36: Hybrid Liquid Solid Phase Synthesis of Incretin Analog from Two Intermediate Fragments Via Native Chemical Ligation

Here, the incretin analog of SEQ ID NO:6 can be made by coupling SEQ ID NOS:25 and 26 via CSPPS essentially as described in Example 35 for coupling SEQ ID NOS:23 and 24.

### Example 36: Synthesis of Compound 35

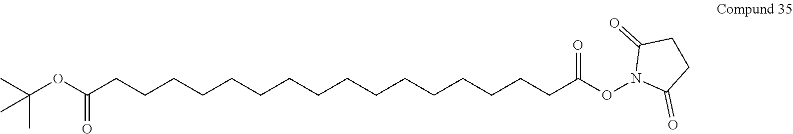 [SMILES: CC(C)(C)OC(=O)CCCCCCCCCCCCCCCCC(=O)ON1C(=O)CCC1=O]

Dichloromethane (7.5 L, 15.0 vol.) is added to a 20 L four-necked flask at 15-30° C., and 18-(tert-butoxy)-18-oxooctadecanoic acid (500.5 g, 1.0 eq. 1.35 mol) and N-hydroxysuccinimide (185.6 g, 1.2 eq., 1.61 mol) are added at 15-30° C. to obtain a suspension. The reaction mixture is cooled to 0-10° C. and charged with N-ethyl-N′-carbodiimide (338.4 g, 1.3 eq., 1.77 mol) in one portion to obtain a solution. The reaction mixture is washed three times with 8 volumes of semi-saturated brine. Compound 35 is used directly in the next step to make Compound 36.

Alternate Method for Compound 35

18-(tert-butoxy)-18-oxooctadecanoic acid (20 g, 53.431 mmol, 99 mass %), N,N′-Disuccinimidyl carbonate (1.2 equiv., 64.117 mmol, 99.6 mass %) and 4-dimethylaminopyridine (0.2 equiv., 1.31 g, 10.7 mmol, 100 mass %) are charged into a 1000 mL baffled, jacketed-reactor equipped with an overhead agitator. Ethyl acetate (800 mL, 40 volumes) is added and the resultant slurry is stirred overnight (18-24 h) at ambient temperature (18° C.-23° C.). ¹H-NMR of the crude sample after 24 h generally shows 97-99% reaction completion. The batch is extracted with de-ionized water (3×82 mL). The organic layer is concentrated in vacuo to ˜160 mL. Ethyl acetate (200 mL) is added to the reaction and the crude reaction solution reduced to ˜180 mL in vacuo at 50° C. The solution is transferred to a jacketed filter and cooled slowly to 3° C., while being agitated. The reaction is then held at 3-5° C. for 1 h. The solids are filtered, washed with cold ethyl acetate (18 mL) and dried under vacuum (7 in of Hg) to give Compound 35 as a solid (22.6 g, 90.5% yield, 99.17% HPLC-CAD).

### Example 37: Synthesis of t-BuO-C18-Glu-1-OtBu (Compound 36)

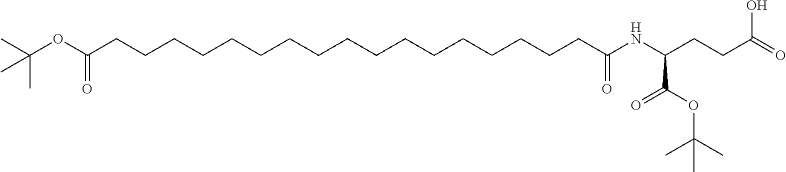 [SMILES: CC(C)(C)OC(=O)CCCCCCCCCCCCCCCCCC(=O)N[C@@H](CCC(=O)O)C(=O)OC(C)(C)C]

Compound 35 is added to a solution of (4S)-4-amino-5-tert-butoxy-5-oxo-pentanoic acid (H-Glu-1-OtBu) (289 g, 1.14 eq., 1 mol) in dichloromethane (2.5 L, 5.0 vol.) in a 20 L four-necked flask at 15-30° C. The reactor is then charged with diisopropylethylamine (230 g, 1.5 eq., 1.78 mol) at 15-30° C., to get a solution. Once Preparation 1<0.5%, the reaction is continued with the next step. The organic phase is washed with 2% aqueous solution of KHSO₄ (4 g/g×3) and concentrated to 1-2 vol. under vacuum at T<50° C. and <−0.08 MPa. Acetonitrile (8 vol.) is charged to the reactor and concentrated to 1-2 vol. at <60° C. Acetonitrile (8 vol.) is charged to the reactor again and concentrated to 5-6 vol. at <60° C. The concentrate is cooled to 40-50° C., stirred for 0.5-1 h and then cooled to 15-30° C. for 2-4 h. The slurry is filtered, washed with acetonitrile (3 vol.) and dried under N₂ to give Compound 36 as a solid (659.5 g, 86.3% yield, 99.1% LCAP).

Alternate Method for Compound 36

Compound 35 (50 g, 104.8 mmol, 98 mass %), (4S)-4-amino-5-tert-butoxy-5-oxo-pentanoic acid H-Glu-1-OtBu (H-Glu-1-OtBu) (25.7 g, 126 mmol, 99.3 mass %) is charged into a 1 L baffled, jacketed-reactor equipped with an overhead agitator and a thermocouple. Acetonitrile (500 mL, 10 V) is used to wash the solids down the funnel into the reaction vessel. Diisopropylethylamine (22 mL, 126 mmol, 99.75 mass %) is then added to the reaction. The reaction is heated to 40° C. and allowed to stir for 18 h. After reaction completion is confirmed by ¹H-NMR/HPLC-CAD, acetic acid (7.2 mL, 130 mmol, 100 mass %) and water (215 mL, 11934.6 mmol, 100 mass %) is charged into the reaction and stirred at 30-35° C. for 1 h. The batch is transferred to a jacketed filter, equipped with an overhead agitator and cooled to −20° C. Solids begin to crystallize out of solution at Tr=2° C. and Tj=−9° C. The solids are held at this temperature for 1 h. De-ionized water (8.6 V, 460 mL) is then poured into this batch and the filter warmed to 0° C. The solids are filtered and dried under high vacuum at 40° C. to give Compound 36 as a solid (55.5 g, 95.3% yield, 99.62% HPLC-CAD).

### Example 38: Synthesis of t-BuO-C18-Glu-1-OtBu-5-ONSu (Compound 37)

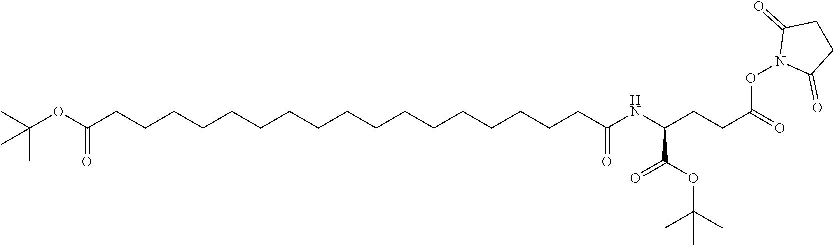 [SMILES: CC(C)(C)OC(=O)CCCCCCCCCCCCCCCCCC(=O)N[C@@H](CCC(=O)ON1C(=O)CCC1=O)C(=O)OC(C)(C)C]

Acetonitrile (12.0 vol.) is added to a 20 L four-necked flask at 15-30° C. Compound 36 (500.4 g, 1.0 eq., 0.90 mol) and N,N′-Disuccinimidyl carbonate (278.5 g, 1.2 eq., 1.09 mol) are added to the flask at 15-30° C. to obtain a suspension. 4-dimethylaminopyridine (11.0 g 0.1 equiv. 0.09 mol) is added in one portion to obtain a solution. Water (1.6 Kg) is added to the over 0.5-1 h. The mixture is cooled to 0-10° C. for 1-2 h, filtered, washed with acetonitrile (2 vol. 0-10° C.) and dried under N₂ to give Compound 37 as a solid (536.0 g, 91.3% yield 100.0% LCAP).

Alternate Synthesis of Compound 37

Compound 36 (55 g, 98.96 mmol), N,N′-Disuccinimidyl carbonate (31 g, 121 mmol, 99.6 mass %), 4-dimethylaminopyridine (1.22 g, 9.89 mmol, 99 mass %) are charged into 1 L baffled, jacketed-reactor equipped with an overhead agitator and a thermocouple. Acetonitrile (660 mL, 12 V) is added to the reaction vessel. The reaction is stirred at 24° C. for 4 h. After reaction completion is confirmed by ¹H-NMR/HPLC-CAD, the reaction solution is transferred to a 1000 mL beaker and de-ionized water (180 mL) is added to the beaker equipped with a magnetic stir bar. Solids crashed out of the reaction as the solution is stirred. The reaction slurry is cooled overnight in the refrigerator (2-10° C.). The solids are filtered and the filter cake, washed with 125 mL cold (2-10° C.) acetonitrile. The solids are dried under high vacuum at 40° C. for 24 h forming Compound 37 (59.3 g, 91.8% yield, 99.61% HPLC-CAD).

### Example 39: Synthesis of t-BuO-C18-Glu-1-OtBu-5-(AEEA)₂ (Compound 38)

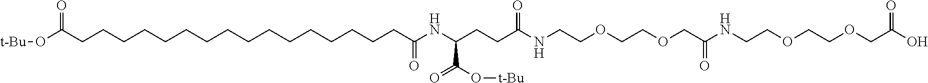 [SMILES: CC(C)(C)OC(=O)CCCCCCCCCCCCCCCCC(=O)N[C@@H](CCC(=O)NCCOCCOCC(=O)NCCOCCOCC(=O)O)C(=O)OC(C)(C)C]

Dichloromethane (7.5 L, 15.0 vol.) is added to a 20 L four-necked flask in one portion at 15-30° C. followed by (AEEA)₂ (261 g, 1.1 eq., 0.85 mol), Compound 37 (501 g, 1.0 eq., 0.77 mol) and diisopropylethylamine (1.5 eq.) at 15-30° C. Once Compound 37 is <0.5%, the reaction is continued with the next step. The reaction mixture is then concentrated to 5-6 vol. under vacuum at T<30° C., P<−0.08 MPa. Ethyl acetate is added to the crude product (5 vol.) and concentrated under vacuum at T<50° C., P<−0.08 MPa. Ethyl acetate (10 vol.) is added to the concentrate and washed with 2% aqueous KHSO₄ (5 g/g×5-6) solution and concentrated to 1.2 vol. under vacuum at T<40° C. and P<−0.0 8 MPa. Dimethylformamide is added to the concentrate (3 g/g vol.) to give the product Compound 38 as a pale yellow solution (2.4 Kg, 92.7% yield, 98.7% LCAP).

Alternate Synthesis of Compound 38

(AEEA)₂ (27.6 g, 1.1 equiv., 85.0 mmol, 95 mass %), N-methyl-N-trimethylsilylacetamide (30 mL, 2 equiv., 200 mmol, 90 mass %) and ethyl acetate (230 mL) are added to a 500 mL flask, equipped with a thermocouple and a magnetic stir bar. The reaction is stirred for 3 h at 18-23° C. After 3 h, Compound 37 (50 g, 76.58 mmol, 100 mass %) and ethyl acetate (130 mL) are added to the reaction flask and stirred for 2 h at 18-23° C. After reaction completion is confirmed by ¹H-NMR/HPLC-CAD, the reaction solution is transferred to a separatory funnel and the organic layer washed with 2% KHSO₄ solution (100 mL×3) and 2% NaCl solution (100 mL×6). The organic layer is concentrated, and the resulting oil is dried under high vacuum at 50° C. for 24 h to give Compound 38 as a waxy solid at −20° C. (66.32 g, 94.9% potency by Q-NMR, 99.53% HPLC-CAD).

### Example 40: Native Chemical Ligation

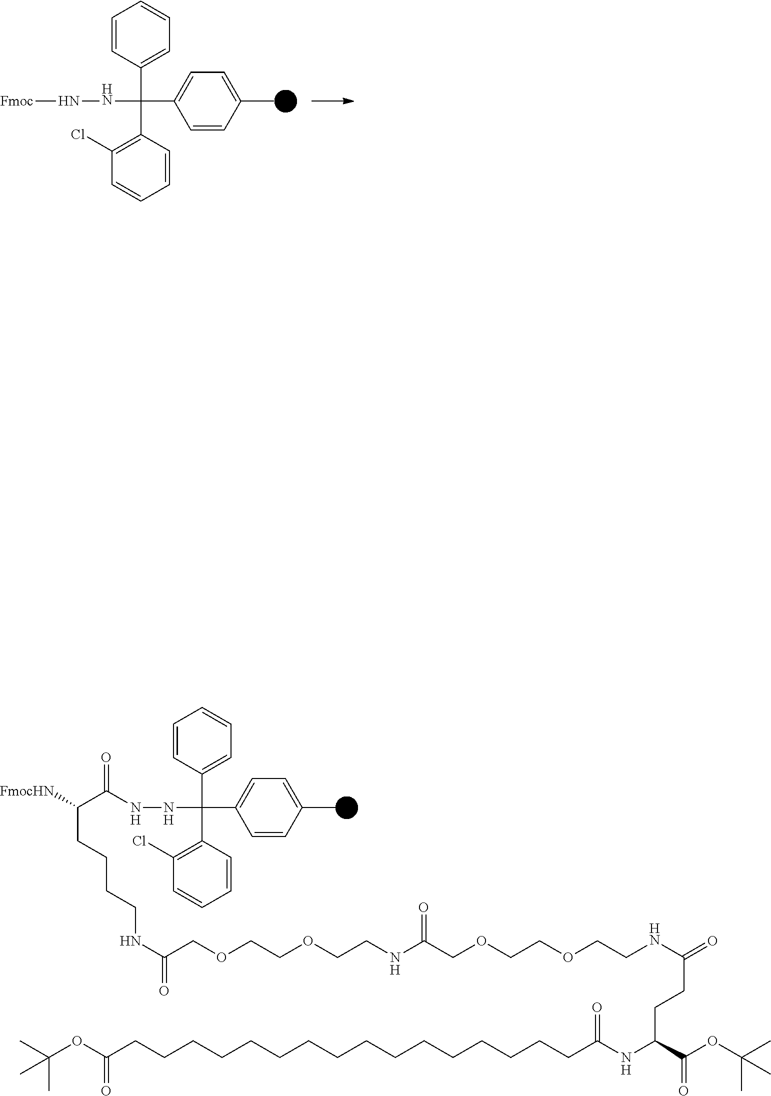

Synthesis of Compound 39

Fmoc-hydrazine-2-chlorotrityl resin (1.16 g, 0.85 mmol) is swollen on a Symphony X synthesizer with 2×10 mL DMF for 20 min each. Fmoc deprotection is performed with 3×10 mL 20% piperidine/DMF for 30 min each. The resin is then washed with 5×10 mL DMF.

(2S)-6-[[2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetyl]amino]-2-(9H-fluoren-9-ylmethoxycarbonylamino)hexanoic acid (Compound 38, 2.13 g, 1.78 mmol, 2.1 equiv) and TNTU (0.715 g, 1.96 mmol, 2.31 equiv) is dissolved in about 20.5 mL of DMF, and N,N-diisopropylethylamine (0.57 mL, 3.27 mmol, 3.85 equiv) is added to the solution. The solution is allowed to mix for 15 min on a rotatory mixer. After 15 min, the solution of pre-activated Compound 38 is added to the resin and the coupling is allowed to run for 8 hours. Then, the resin is washed with 5×10 mL DMF, 5×10 mL DCM and dried for 8 hours on the synthesizer. The resin loading is determined to be 0.45 mmol/g by quantitative NMR.

### Example 41: Synthesis of Compound 40 (SEQ ID NO:58)

About 1.10 g each of Compound 39 (loading value: 0.45 mmol/g) are added in two 40 mL reactor vessels and swollen with 2×20 mL DMF for 20 min each. SEQ ID NO:58 is synthesized using standard SPPS protocols.

Deprotection: 4×9 mL of 20% v/v piperidine in DMF, 30 minutes each.

Couplings: 3 equivalents of amino acid, 3 equivalents of OXYMA and 3.3 equivalents of DIC are used for amino acid coupling.

During SPPS, the resin is washed with 5×9 mL DMF with 1 min N₂ mix after each coupling and the final iteration of fmoc deprotection. At the end of the peptide hydrazide synthesis, the resin is washed with DCM with N₂ mixing. The resin is dried on the peptide synthesizer.

Global Deprotection and Cleavage

45 mL of the cleavage cocktail made with 2.5% w/v dithiothreitol (DTT), 2.5% v/v water, 2.5% v/v triisopropylsilane (TIPS) and 92.5% trifluoroacetic acid (TFA) is added to the dried resin (4.2 g) in a 500 mL three-necked round bottom flask and stirred for about 3 hours. The resin is filtered and washed with 2×2.5 mL TFA. The filtrate is poured into 350 mL cold MTBE and the peptide precipitated out immediately. The filtration flask is washed with 2×2.5 mL TFA and poured into the cold MTBE. It is cooled down to −20° C. for half an hour and then centrifuged. The peptide precipitate is then washed twice with 300 mL MTBE and centrifuged. The peptide precipitate is dried in a vacuum oven at 27° C. for about 14 hours. About 2.75 g of the crude Compound 40 is obtained after drying [Expected (mass+2H⁺)/2=1356.2257, observed (mass+2H⁺)/2=1356.2245].

### Example 42: Synthesis of Compound 41 (SEQ ID NO:59)

About 0.50 mmol of Compound 41 (SEQ ID NO:59) is synthesized on Sieber amide resin by standard SPPS protocols similar to the synthesis of Compound 40, SEQ ID NO:58.

Global Deprotection and Cleavage: 25 mL of the cleavage cocktail made with 5% w/v dithiothreitol (DTT), 2.5% v/v water, 2.5% v/v triisopropylsilane (TIPS) and 90% trifluoroacetic acid (TFA) is added to the dried resin (2.21 g) and mixed for 3 hours on a rotary mixer. The resin is filtered and washed with 2×2.0 mL TFA. The filtrate is poured into 175. mL cold MTBE and peptide precipitated out immediately. The filtration flask is washed with 2×2 mL TFA and is poured into the cold MTBE. It is cooled down to −20° C. for 30 min and then centrifuged. The peptide precipitate is washed twice with 150 mL MTBE and centrifuged. The peptide precipitate is dried in a vacuum oven at 27° C. for 14 hours. About 1.351 g of the crude Compound 41 (SEQ ID NO:58) is obtained after drying. It is purified by RP-HPLC on a Waters CSH C18 10 um column (10 mm×250 mm) at the ambient temperature with a linear gradient of 15-35% acetonitrile in water (0.1% TFA) over 23 min after 10% acetonitrile in water (0.1% TFA) for the first 3 min and 10-15% acetonitrile in water (0.1% TFA) from 3 to 5 min. About 650 mg of the purified Compound 41 is obtained [Expected (mass+2H⁺)/2=1138.5486, observed (mass+2H⁺)/2=1138.5458].

### Example 43: Synthesis of Compound 42 (SEQ ID NO:60)

Compound 42 (Thioester synthesis), SEQ ID NO:60

Crude peptide hydrazide (Compound 40; SEQ ID NO:58, 118.2 mg, 0.044 mmol) is dissolved in 10 mL of the ligation buffer (6 M guanidine hydrochloride and 0.3 M sodium hydrogen phosphate monobasic, pH about 3.5). The solution is cooled to −15° C. in an acetone-ice bath. 0.3 mL of 1 M sodium nitrite solution (20.7 mg, 0.3 mmol, 6.8 equiv.) is added to the peptide hydrazide solution and allowed to stir for 15 min at −15° C. Meanwhile, 0.2 mL thiophenol is diluted to 1.1 mL with the ligation buffer (pH about 7.0). After 15 min, 1.1 mL of the thiophenol mixture is added to the peptide hydrazide solution to cause in-situ thiolysis of the peptidyl azide generated from Compound 40.

The pH of the reaction mixture is adjusted to about 7.0 with 5 N sodium hydroxide solution. Thiolysis of the peptidyl azide is allowed to run for 15 min to give Compound 42 (SEQ ID NO:60).

### Example 44: Compound 43, SEQ ID NO:61 (Native Chemical Ligation with the Thioester Compound 42)

Compound 41 (SEQ ID NO:59 (75.4 mg, 0.033 mmol) is dissolved in 2 mL of the ligation buffer (pH about 7.0) in a scintillation vial and the solution is added to the crude thioester solution Compound 42 (SEQ ID NO:60). The vial is rinsed with 1 mL of the ligation buffer (pH about 7.0) and the rinse is added to the reaction mixture. 1.5 mL of tris(2-carboxyethyl)phosphine (TCEP, 0.25 M in the ligation buffer, pH about 7.0) and 1.0 mL of ascorbic acid solution (0.53 M in the ligation buffer, pH about 7.0) are added to the reaction mixture. The pH of the reaction mixture is adjusted to about 7.1 with 5 N NaOH solution and the solution turned clear. The reaction is complete in 9-10 hours to yield SEQ ID NO:61.

### Example 45: Synthesis of SEQ ID NO:62 (Compound 44)

TABLE 25
SPPS Conditions for SEQ ID NO: 62 (Compound 44).

| | | | SPPS Conditions |
|---|---|---|---|
| Cycle | Deprotection | Amino Acid | Solvent for Couplings: DMF |
| 1 | 2 × 5-30 min | Fmoc-Gly-OH | 2.0-3.0 AA/2.2-3.3 DIC/2.0-3.0 |
| | 20% Pip/DMF | | Oxyma |
| | | | 4-15 hrs, rt |
| 2 | 2 × 5-30 min | Fmoc-Glu(OtBu)-OH | 2.0-3.0 AA/2.2-3.3 DIC/2.0-3.0 |
| | 20% Pip/DMF | | Oxyma |
| | | | 4-15 hrs, rt |
| 3 | 2 × 5-30 min | Fmoc-Ile-OH | 2.0-3.0 AA/2.2-3.3 DIC/2.0-3.0 |
| | 20% Pip/DMF | | Oxyma |
| | | | 4-15 hrs, rt |
| 4 | 2 × 5-30 min | Fmoc-Leu-OH | 2.0-3.0 AA/2.2-3.3 DIC/2.0-3.0 |
| | 20% Pip/DMF | | Oxyma |
| | | | 4-15 hrs, rt |
| 5 | 2 × 5-30 min | Fmoc-2-MeTyr(tBu)-OH | 1.5-3.0 AA/1.7-3.3 DIC/1.5-3.0 |
| | 20% Pip/DMF | | Oxyma |
| | | | 4-15 hrs, rt |
| 6 | 2 × 5-30 min | Fmoc-D-Glu(OtBu)-OH | 2.0-3.0 AA/2.2-3.3 DIC/2.0-3.0 |
| | 20% Pip/DMF | | Oxyma |
| | | | 4-15 hrs, rt |
| 7 | 2 × 5-30 min | Fmoc-Ile-OH | 2.0-3.0 AA/2.2-3.3 DIC/2.0-3.0 |
| | 20% Pip/DMF | | Oxyma |
| | | | 4-15 hrs, rt |
| 8 | 2 × 5-30 min | Fmoc-Phe-OH | 2.0-3.0 AA/2.2-3.3 DIC/2.0-3.0 |
| | 20% Pip/DMF | | Oxyma |
| | | | 4-15 hrs, rt |
| 9 | 2 × 5-30 min | Fmoc-Glu(OtBu)-OH | 2.0-3.0 AA/2.2-3.3 DIC/2.0-3.0 |
| | 20% Pip/DMF | | Oxyma |
| | | | 4-15 hrs, rt |
| 10 | 2 × 5-30 min | Fmoc-Aib-OH | 2.0-3.0 AA/2.2-3.3 DIC/2.0-3.0 |
| | 20% Pip/DMF | | Oxyma |
| | | | 4-15 hrs, rt |
| 11 | 2 × 5-30 min | Fmoc-Gln(Trt)-OH | 2.0-3.0 AA/2.2-3.3 DIC/2.0-3.0 |
| | 20% Pip/DMF | | Oxyma |
| | | | 4-15 hrs, rt |

Fragment Cleavage and Isolation: Fragment on CTC resin is swelled twice using DCM. A reactor with resin is cooled to about 15° C., and 2% TFA/DCM (4 ml/g of resin) is charged to the reactor and then stirred for 15 minutes under nitrogen. 1% TFA/DCM (4 ml/g of resin) is then charged and allowed to stir for 15 minutes and after filtering this is repeated. The resin is filtered and washed with 3×10 V of DCM. All the filtrates are combined together and neutralized with DIPEA. DCM is removed from the resulting solution and brine is charged to precipitate fragment 2. Then, the resulting slurry is filtered while maintaining temperature at about 15° C. To the cake, 14 V of fresh MTBE is added, is stirred for 30 min at about 15° C., and then is filtered. Washing is repeated one more time, and the resulting light-yellow solid is dried at about 35° C.

### Example 46: Synthesis of SEQ ID NO:42 (Compound 45)

TABLE 26
SPPS Conditions for the synthesis of SEQ ID NO: 42 (Compound 45).

| | | | SPPS Conditions |
|---|---|---|---|
| Cycle | Deprotection | Amino Acid | Solvent for Couplings: DMF |
| 1 | 2 × 5-30 min 20% | Fmoc-Ala-OH | 2.0-3.0 AA/2.2-3.3 DIC/2.0-3.0 |
| | Pip/DMF | | Oxyma |
| | | | 4-15 hrs, rt |
| 2 | 2 × 5-30 min 20% | Fmoc-Lys(Mtt)-OH | 2.0-3.0 AA/2.2-3.3 DIC/2.0-3.0 |
| | Pip/DMF | | Oxyma |
| | | | 4-15 hrs, rt |
| 3 | 2 × 5-30 min 20% | Fmoc-Orn(Boc)-OH | 2.0-3.0 AA/2.2-3.3 DIC/2.0-3.0 |
| | Pip/DMF | | Oxyma |
| | | | 4-15 hrs, rt |
| 4 | 2 × 5-30 min 20% | Fmoc-Asp(OtBu)-OH | 2.0-3.0 AA/2.2-3.3 DIC/2.0-3.0 |
| | Pip/DMF | | Oxyma |
| | | | 4-15 hrs, rt |
| 5 | HFIP/DCM | Compound 38 | 1.5-3.0 AA/1.7-3.3 DIC/1.5-3.0 |
| | | | Oxyma |
| | | | 4-15 hrs, rt |

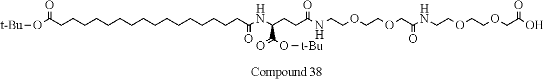 [SMILES: CC(C)(C)OC(=O)CCCCCCCCCCCCCCCCC(=O)N[C@@H](CCC(=O)NCCOCCOCC(=O)NCCOCCOCC(=O)O)C(=O)OC(C)(C)C]

Fragment Cleavage and Isolation: Fragment on CTC resin is swelled twice using DCM. A reactor with resin is cooled to about 15° C., and 20% HFIP/DCM (8 ml/g of resin) is charged to the reactor and then stirred for 60 minutes under nitrogen. 20% HFIP/DCM (4 ml/g of resin) is then charged and allowed to stir for 60 minutes. The resin is filtered and washed with 3×10 V of DCM. All the filtrates are combined and DCM and HFIP are removed from the resulting and heptane and ether is charged to precipitate fragment 3. Then, the resulting slurry is filtered while maintaining temperature at about 15° C. To the cake, 14 V of fresh MTBE is added, is stirred for 30 min at about 15° C., and then is filtered. Washing is repeated one more time, and the resulting orange solid is dried at about 35° C.

### Example 47: Synthesis of SEQ ID NO:31 (Compound 28)

TABLE 27
SPPS Conditions for the synthesis of SEQ ID NO: 31.

| | | | SPPS Conditions |
|---|---|---|---|
| Cycle | Deprotection | Amino Acid | Solvent for Couplings: DMF |
| 1 | 2 × 5-30 min | Fmoc-Leu-OH | 2.0-3.0 AA/2.2-3.3 DIC/2.0-3.0 |
| | 20% Pip/DMF | | Oxyma |
| | | | 4-15 hrs, rt |
| 2 | 2 × 5-30 min | Fmoc-2-MeLeu-OH | 2.0-3.0 AA/2.2-3.3 DIC/2.0-3.0 |
| | 20% Pip/DMF | | Oxyma |
| | | | 4-15 hrs, rt |
| 3 | 2 × 5-30 min | Fmoc-Ile-OH | 2.0-3.0 AA/2.2-3.3 DIC/2.0-3.0 |
| | 20% Pip/DMF | | Oxyma |
| | | | 4-15 hrs, rt |
| 4 | 2 × 5-30 min | Fmoc-Ser(tBu)-OH | 2.0-3.0 AA/2.2-3.3 DIC/2.0-3.0 |
| | 20% Pip/DMF | | Oxyma |
| | | | 4-15 hrs, rt |
| 5 | 2 × 5-30 min | Fmoc-4Pal-OH | 1.5-3.0 AA/1.7-3.3 DIC/1.5-3.0 |
| | 20% Pip/DMF | | Oxyma |
| | | | 4-15 hrs, rt |
| 6 | 2 × 5-30 min | Fmoc-Asp(OtBu)-OH | 2.0-3.0 AA/2.2-3.3 DIC/2.0-3.0 |
| | 20% Pip/DMF | | Oxyma |
| | | | 4-15 hrs, rt |
| 7 | 2 × 5-30 min | Fmoc-Ser(tBu)-OH | 2.0-3.0 AA/2.2-3.3 DIC/2.0-3.0 |
| | 20% Pip/DMF | | Oxyma |
| | | | 4-15 hrs, rt |
| 8 | 2 × 5-30 min | Fmoc-Thr(tBu)-OH | 2.0-3.0 AA/2.2-3.3 DIC/2.0-3.0 |
| | 20% Pip/DMF | | Oxyma |
| | | | 4-15 hrs, rt |
| 9 | 2 × 5-30 min | Fmoc-2-MePhe(2F)-OH | 1.5-3.0 AA/1.7-3.3 DIC/1.5-3.0 |
| | 20% Pip/DMF | | Oxyma |
| | | | 4-15 hrs, rt |
| 10 | 2 × 5-30 min | Fmoc-Thr(tBu)-OH | 2.0-3.0 AA/2.2-3.3 DIC/2.0-3.0 |
| | 20% Pip/DMF | | Oxyma |
| | | | 4-15 hrs, rt |
| 11 | 2 × 5-30 min | Fmoc-Gly-OH | 2.0-3.0 AA/2.2-3.3 DIC/2.0-3.0 |
| | 20% Pip/DMF | | Oxyma |
| | | | 4-15 hrs, rt |
| 12 | 2 × 5-30 min | Fmoc-Glu(OtBu)-OH | 2.0-3.0 AA/2.2-3.3 DIC/2.0-3.0 |
| | 20% Pip/DMF | | Oxyma |
| | | | 4-15 hrs, rt |
| 13 | 2 × 5-30 min | Fmoc-Tyr(tBu)-Aib- | 1.5-3.0 AA/1.7-3.3 DIC/1.5-3.0 |
| | 20% Pip/DMF | OH | Oxyma |
| | | | 4-15 hrs, rt |
| 14 | | (Boc)2O | |

Fragment Cleavage and Isolation: Fragment on CTC resin is swelled twice using DCM. A reactor with resin is cooled to about 15° C., and 2% TFA/DCM (4 ml/g of resin) is charged to the reactor and then stirred for 15 minutes under nitrogen. 1% TFA/DCM (4 ml/g of resin) is then charged and allowed to stir for 15 minutes and after filtering this is repeated. The resin is filtered and washed with 3×10 V of DCM. All the filtrates are combined together and neutralized with DIPEA. DCM is removed from the resulting solution and brine is charged to precipitate fragment 2. Then, the resulting slurry is filtered while maintaining temperature at about 15° C. To the cake, 14 V of fresh MTBE is added, is stirred for 30 min at about 15° C., and then is filtered. Washing is repeated one more time, and the resulting off-white solid is dried at about 35° C.

### Example 48: Synthesis of SEQ ID NO:43 (Compound 46)

TABLE 28
Preparation of SEQ ID NO: 43 via SPPS:

| | | | SPPS Conditions |
|---|---|---|---|
| Cycle | Deprotection | Amino Acid | Solvent for Couplings: DMF |
| 1 | 2 × 5-30 min | Fmoc-L-Ser(t-Bu)-OH | 2.0-3.0 AA/2.2-3.3 DIC/2.0-3.0 |
| | 20% Pip/DMF | | Oxyma |
| | | | 4-15 hrs, rt |
| 2 | 2 × 5-30 min | Fmoc-L-Pro-OH | 2.0-3.0 AA/2.2-3.3 DIC/2.0-3.0 |
| | 20% Pip/DMF | | Oxyma |
| | | | 4-15 hrs, rt |
| 3 | 2 × 5-30 min | Fmoc-L-Pro-OH | 2.0-3.0 AA/2.2-3.3 DIC/2.0-3.0 |
| | 20% Pip/DMF | | Oxyma |
| | | | 4-15 hrs, rt |
| 4 | 2 × 5-30 min | Fmoc-L-Pro-OH | 2.0-3.0 AA/2.2-3.3 DIC/2.0-3.0 |
| | 20% Pip/DMF | | Oxyma |
| | | | 4-15 hrs, rt |
| 3-4* | Alternatively use Fmoc-L-Pro-Pro-OH dimer instead of step 3 & 4 | | |
| 5 | 2 × 5-30 min | Fmoc-L-Ala-OH | 2.0-3.0 AA/2.2-3.3 DIC/2.0-3.0 |
| | 20% Pip/DMF | | Oxyma |
| | | | 4-15 hrs, rt |
| 6 | 2 × 5-30 min | Fmoc-Gly-OH | 2.0-3.0 AA/2.2-3.3 DIC/2.0-3.0 |
| | 20% Pip/DMF | | Oxyma |
| | | | 4-15 hrs, rt |
| 7 | 2 × 5-30 min | Fmoc-L-Ser(t-Bu)-OH | 2.0-3.0 AA/2.2-3.3 DIC/2.0-3.0 |
| | 20% Pip/DMF | | Oxyma |
| | | | 4-15 hrs, rt |
| 8 | 2 × 5-30 min | Fmoc-L-Ser(t-Bu)-OH | 2.0-3.0 AA/2.2-3.3 DIC/2.0-3.0 |
| | 20% Pip/DMF | | Oxyma |
| | | | 4-15 hrs, rt |
| 9 | 2 × 5-30 min | Fmoc-L-Pro-OH | 2.0-3.0 AA/2.2-3.3 DIC/2.0-3.0 |
| | 20% Pip/DMF | | Oxyma |
| | | | 4-15 hrs, rt |
| 10 | 2 × 5-30 min | Fmoc-Gly-OH | 2.0-3.0 AA/2.2-3.3 DIC/2.0-3.0 |
| | 20% Pip/DMF | | Oxyma |
| | | | 4-15 hrs, rt |
| 11 | 2 × 5-30 min | Fmoc-Gly-OH | 2.0-3.0 AA/2.2-3.3 DIC/2.0-3.0 |
| | 20% Pip/DMF | | Oxyma |
| | | | 4-15 hrs, rt |
| 12 | 2 × 5-30 min | Fmoc-Glu(OtBu)-OH | 2.0-3.0 AA/2.2-3.3 DIC/2.0-3.0 |
| | 20% Pip/DMF | | Oxyma |
| | | | 4-15 hrs, rt |
| 13 | 2 × 5-30 min | Fmoc-Ile-OH | 2.0-3.0 AA/2.2-3.3 DIC/2.0-3.0 |
| | 20% Pip/DMF | | Oxyma |
| | | | 4-15 hrs, rt |
| 14 | 2 × 5-30 min | Fmoc-Leu-OH | 2.0-3.0 AA/2.2-3.3 DIC/2.0-3.0 |
| | 20% Pip/DMF | | Oxyma |
| | | | 4-15 hrs, rt |
| 15 | 2 × 5-30 min | Fmoc-2-MeTyr(tBu)-OH | 1.5-3.0 AA/1.7-3.3 DIC/1.5-3.0 |
| | 20% Pip/DMF | | Oxyma |
| | | | 4-15 hrs, rt |
| 16 | 2 × 5-30 min | Fmoc-D-Glu(OtBu)-OH | 2.0-3.0 AA/2.2-3.3 DIC/2.0-3.0 |
| | 20% Pip/DMF | | Oxyma |
| | | | 4-15 hrs, rt |
| 17 | 2 × 5-30 min | Fmoc-Ile-OH | 2.0-3.0 AA/2.2-3.3 DIC/2.0-3.0 |
| | 20% Pip/DMF | | Oxyma |
| | | | 4-15 hrs, rt |
| 18 | 2 × 5-30 min | Fmoc-Phe-OH | 2.0-3.0 AA/2.2-3.3 DIC/2.0-3.0 |
| | 20% Pip/DMF | | Oxyma |
| | | | 4-15 hrs, rt |
| 19 | 2 × 5-30 min | Fmoc-Glu(OtBu)-OH | 2.0-3.0 AA/2.2-3.3 DIC/2.0-3.0 |
| | 20% Pip/DMF | | Oxyma |
| | | | 4-15 hrs, rt |
| 20 | 2 × 5-30 min | Fmoc-Aib-OH | 2.0-3.0 AA/2.2-3.3 DIC/2.0-3.0 |
| | 20% Pip/DMF | | Oxyma |
| | | | 4-15 hrs, rt |
| 21 | 2 × 5-30 min | Fmoc-Gln(Trt)-OH | 2.0-3.0 AA/2.2-3.3 DIC/2.0-3.0 |
| | 20% Pip/DMF | | Oxyma |
| | | | 4-15 hrs, rt |

Fragment Cleavage and Isolation: Fragment on Sieber resin is swelled twice using 10 V DCM for 10-20 min. A reactor with resin is cooled to about 15° C., and washed sequentially with 6% TFA/DCM (5 ml/g of resin) for 15 min, 3% TFA/DCM (5 ml/g of resin) for 15 min, 1% TFA/DCM (10 ml/g of resin) for 5 min, 1% TFA/DCM (5 ml/g of resin) for 3 min and 1% TFA/DCM (2.5 ml/g of resin) for 3 min. The resin is filtered and washed with 3×10 V of DCM. All the filtrates are combined together. DCM is removed from the resulting solution under reduced pressure and reconstituted with EtOAc. Heptane is charged to the solution and the resulting slurry is filtered while maintaining temperature at about 15° C. To the cake, 14 V of fresh MTBE is added, is stirred for 30 min at about 15° C., and then is filtered. Washing is repeated one more time, and the resulting off-white solid is dried at about 35° C.

### Example 49: Synthesis of SEQ ID NO:44 (Compound 47)

TABLE 29
Preparation of SEQ ID NO: 44.

| | | | SPPS Conditions |
|---|---|---|---|
| Cycle | Deprotection | Amino Acid | Solvent for Couplings: DMF |
| 1 | 2 × 5-30 min | Fmoc-Ala-OH | 2.0-3.0 AA/2.2-3.3 DIC/2.0-3.0 |
| | 20% Pip/DMF | | Oxyma |
| | | | 4-15 hrs, rt |
| 2 | 2 × 5-30 min | Fmoc-Lys(Mtt)-OH | 2.0-3.0 AA/2.2-3.3 DIC/2.0-3.0 |
| | 20% Pip/DMF | | Oxyma |
| | | | 4-15 hrs, rt |
| 3 | 2 × 5-30 min | Fmoc-Orn(Boc)-OH | 2.0-3.0 AA/2.2-3.3 DIC/2.0-3.0 |
| | 20% Pip/DMF | | Oxyma |
| | | | 4-15 hrs, rt |
| 4 | 2 × 5-30 min | Fmoc-Asp(OtBu)-OH | 2.0-3.0 AA/2.2-3.3 DIC/2.0-3.0 |
| | 20% Pip/DMF | | Oxyma |
| | | | 4-15 hrs, rt |
| 5 | HFIP/DCM | Compound 38 | 1.5-3.0 AA/1.7-3.3 DIC/1.5-3.0 |
| | | | Oxyma |
| | | | 4-15 hrs, rt |
| 6 | 2 × 5-30 min | Fmoc-Leu-OH | 2.0-3.0 AA/2.2-3.3 DIC/2.0-3.0 |
| | 20% Pip/DMF | | Oxyma |
| | | | 4-15 hrs, rt |
| 7 | 2 × 5-30 min | Fmoc-2-MeLeu-OH | 2.0-3.0 AA/2.2-3.3 DIC/2.0-3.0 |
| | 20% Pip/DMF | | Oxyma |
| | | | 4-15 hrs, rt |
| 8 | 2 × 5-30 min | Fmoc-Ile-OH | 2.0-3.0 AA/2.2-3.3 DIC/2.0-3.0 |
| | 20% Pip/DMF | | Oxyma |
| | | | 4-15 hrs, rt |
| 9 | 2 × 5-30 min | Fmoc-Ser(tBu)-OH | 2.0-3.0 AA/2.2-3.3 DIC/2.0-3.0 |
| | 20% Pip/DMF | | Oxyma |
| | | | 4-15 hrs, rt |
| 10 | 2 × 5-30 min | Fmoc-4Pal-OH | 1.5-3.0 AA/1.7-3.3 DIC/1.5-3.0 |
| | 20% Pip/DMF | | Oxyma |
| | | | 4-15 hrs, rt |
| 11 | 2 × 5-30 min | Fmoc-Asp(OtBu)-OH | 2.0-3.0 AA/2.2-3.3 DIC/2.0-3.0 |
| | 20% Pip/DMF | | Oxyma |
| | | | 4-15 hrs, rt |
| 12 | 2 × 5-30 min | Fmoc-Ser(tBu)-OH | 2.0-3.0 AA/2.2-3.3 DIC/2.0-3.0 |
| | 20% Pip/DMF | | Oxyma |
| | | | 4-15 hrs, rt |
| 13 | 2 × 5-30 min | Fmoc-Thr(tBu)-OH | 2.0-3.0 AA/2.2-3.3 DIC/2.0-3.0 |
| | 20% Pip/DMF | | Oxyma |
| | | | 4-15 hrs, rt |
| 14 | 2 × 5-30 min | Fmoc-2-MePhe(2F)—OH | 1.5-3.0 AA/1.7-3.3 DIC/1.5-3.0 |
| | 20% Pip/DMF | | Oxyma |
| | | | 4-15 hrs, rt |
| 15 | 2 × 5-30 min | Fmoc-Thr(tBu)-OH | 2.0-3.0 AA/2.2-3.3 DIC/2.0-3.0 |
| | 20% Pip/DMF | | Oxyma |
| | | | 4-15 hrs, rt |
| 16 | 2 × 5-30 min | Fmoc-Gly-OH | 2.0-3.0 AA/2.2-3.3 DIC/2.0-3.0 |
| | 20% Pip/DMF | | Oxyma |
| | | | 4-15 hrs, rt |
| 17 | 2 × 5-30 min | Fmoc-Glu(OtBu)-OH | 2.0-3.0 AA/2.2-3.3 DIC/2.0-3.0 |
| | 20% Pip/DMF | (H2O) | Oxyma |
| | | | 4-15 hrs, rt |
| 18 | 2 × 5-30 min | Fmoc-Tyr(tBu)-Aib-OH | 1.5-3.0 AA/1.7-3.3 DIC/1.5-3.0 |
| | 20% Pip/DMF | | Oxyma |
| | | | 4-15 hrs, rt |
| 19 | | (Boc)2O | |

Fragment Cleavage and Isolation: Fragment on Sieber resin is swelled twice using 10 V DCM for 10-20 min. A reactor with resin is cooled to about 15° C., and washed sequentially with 6% TFA/DCM (5 ml/g of resin) for 15 min, 3% TFA/DCM (5 ml/g of resin) for 15 min, 1% TFA/DCM (10 ml/g of resin) for 5 min, 1% TFA/DCM (5 ml/g of resin) for 3 min and 1% TFA/DCM (2.5 ml/g of resin) for 3 min. The resin is filtered and washed with 3×10 V of DCM. All the filtrates are combined together. DCM is removed from the resulting solution under reduced pressure and reconstituted with EtOAc. Heptane is charged to the solution and the resulting slurry is filtered while maintaining temperature at about 15° C. To the cake, 14 V of fresh MTBE is added, is stirred for 30 min at about 15° C., and then is filtered. Washing is repeated one more time, and the resulting off-white solid is dried at about 35° C.

### Example 50: Preparation of SEQ ID NO:29

Hybrid Liquid Solid Phase Synthesis of SEQ ID NO:29 from Four Intermediate Fragments Via Chemical Conjugation.

SEQ ID NO:29 can be made by coupling SEQ ID NOs:7, 42, 31 and 62 via HLSPS. SEQ ID NO's:7 and 62 are coupled to produce SEQ ID NO:43. SEQ ID NO's:42 and 31 are coupled to produce SEQ ID NO:44. SEQ ID NO:43 and SEQ ID NO:44 are coupled to produce SEQ ID NO:29.

Briefly, a solution of SEQ ID NO:7 (1.00 mmol) and a solution of SEQ ID NO:62 (1.1 mmol) in 10 V of DMSO are coupled using PyBOP, DEPBT or EDC/HONB reagent (1.30-2.00 mmol) and DIEA (2 mmol) at rt. The mixture is stirred at rt until the reaction is deemed complete by HPLC. The mixture is quenched with 20 V of 15-20% brine solution, and then an additional 10 V of water is added and is stirred for 10 min. The resulting slurry is filtered, and the solid is washed with 3×10 V of water. The solid is dried in a vacuum dryer (40° C.) to yield SEQ ID NO:43 as an off-white solid.

Next, a solution of SEQ ID NO:31 (1.00 mmol) and a solution of SEQ ID NO:42 (1.1 mmol) in x V of DMSO are coupled using PyBOP, DEPBT or EDC/HONB reagent (1.30-2.00 mmol) and DIEA (2 mmol) at rt. The mixture is stirred at rt until the reaction is deemed complete by HPLC. The mixture is quenched with 20 V of 15-20% brine solution, and then an additional 10 V of water is added and is stirred for 10 min. The resulting slurry is filtered, and the solid is washed with 3×10 V of water. The solid is dried in a vacuum dryer (40° C.) to yield SEQ ID NO:44 as an off-white solid.

Finally, a solution of SEQ ID NO:43 (1.00 mmol) and a solution of SEQ ID NO:44 (1.0 mmol) in 17 V of THF are coupled using DEPBT reagent (1.30-2.00 mmol) and DIEA (1 mmol) at rt. The mixture is stirred at rt until the reaction is deemed complete by HPLC. The mixture is quenched with 20 V of water. The resulting slurry is filtered, and the solid is washed with 3×10 V of water. The solid is dried in a vacuum dryer (40° C.) to yield product as a white solid. The resulting peptide is deprotected by with a cocktail of TFA:TIPS:DTT:water (92.5:2.5:2.5:2.5) with 10 ml per g of starting material. After stirring for 3 hours at room temperature, the product is precipitated with 4 mL of 20% Heptane/MTBE per mL of cocktail while keeping the temperature below 30° C. The resulting slurry is filtered, and the solid is washed with 3×10 V of MTBE. The solid is dried in a vacuum dryer (40° C.) to yield product as an off-white solid.

### Example 51: Alternative Synthesis of SEQ ID NO:7

TABLE 30
The synthesis uses Fmoc-Sieber amide resin with
a loading of 0.80 mmol/g. The general SPPS procedure
is used with the following modifications:

| | | SPPS conditions |
|---|---|---|
| Cycle | Amino acid | Solvent for couplings: DMF |
| 1 | Fmoc-L-Ser(t-Bu)-OH | 2 × 30 min De-Fmoc cycles, |
| | | 6 × 2 min post-dep washes, |
| | | 2.0 AA/2.2 DIC/2.0 Oxyma |
| | | 4 h, rt. |
| 2 | Fmoc-L-Pro-OH | 2 × 30 min De-Fmoc cycles, |
| | | 6 × 2 min post-dep washes, |
| | | Double coupling: |
| | | (1.5 AA/1.65 DIC/1.5 Oxyma) × 2 |
| | | 3 + 16 h, rt. |
| 3 | Fmoc-L-Pro-OH | 3 × 30 min De-Fmoc cycles, |
| | | 6 × 2 min post-dep washes, |
| | | Double coupling: |
| | | (1.5 AA/1.65 DIC/1.5 Oxyma) × 2 |
| | | 3 + 16 h, rt. |
| 4 | Fmoc-L-Pro-OH | 3 × 30 min De-Fmoc cycles, |
| | | 6 × 2 min post-dep washes, |
| | | Double coupling: |
| | | (1.5 AA/1.65 DIC/1.5 Oxyma) × 2 |
| | | 3 + 16 h, rt. |
| 5 | Fmoc-L-Ala-OH | 3 × 30 min De-Fmoc cycles, |
| | | 6 × 2 min post-dep washes, |
| | | Double coupling: |
| | | (1.5 AA/1.65 DIC/1.5 Oxyma) × 2 |
| | | 3 + 16 h, rt. |
| 6 | Fmoc-Gly-OH | 2 × 30 min De-Fmoc cycles, |
| | | 6 × 2 min post-dep washes, |
| | | 2.0 AA/2.2 DIC/2.0 Oxyma |
| | | 4 h, rt. |
| 7 | Fmoc-L-Ser(t-Bu)-OH | 2 × 30 min De-Fmoc cycles, |
| | | 6 × 2 min post-dep washes, |
| | | 2.0 AA/2.2 DIC/2.0 Oxyma |
| | | 4 h, rt. |
| 8 | Fmoc-L-Ser(t-Bu)-OH | 2 × 30 min De-Fmoc cycles, |
| | | 6 × 2 min post-dep washes, |
| | | 2.0 AA/2.2 DIC/2.0 Oxyma |
| | | 6 h, rt |
| 9 | Fmoc-L-Pro-OH | 2 × 30 min De-Fmoc cycles, |
| | | 6 × 2 min post-dep washes, |
| | | 2.0 AA/2.2 DIC/2.0 Oxyma |
| | | 6 h, rt |
| 10 | Fmoc-Gly-OH | 3 × 30 min De-Fmoc cycles, |
| | | 6 × 2 min post-dep washes, |
| | | 2.0 AA/2.2 DIC/2.0 Oxyma |
| | | 10 h, rt. |

The resin bound SEQ ID NO:7 (54 g, ˜24.0 mmol) is treated with 2×300 mL (30 min each) of 20% Pip/DMF. 2) Wash with 6×300 mL of DMF followed by 5×300 mL of DCM. 3) Add 500 mL TFA/DCM (5/95, v/v) and stir for 2 h. 4) Filter the mixtures and wash with 500 mL of DCM, to give a total filtrate volume of 1000 mL. 5) Concentrate to ˜250 mL. 6) Charge 250 mL MTBE. 7) Repeat step 5-6 for 5-6 times. 8) Filter and collect wet cake, and dry in a vacuum oven at 33° C. overnight to produce SEQ ID NO:7 (18.3 g, 75% yield) of a white solid. Analysis of the isolated solid using UPLC (94.4 area %). LC-MS ([M+H]⁺): 1020.58.

### Example 52: Synthesis of SEQ ID NO:45 (Compound 48)

TABLE 31
The synthesis uses 2-CTC resin with a loading of 0.80 mmol/g. The
general SPPS procedure is used with the following modifications:

| | | SPPS conditions |
|---|---|---|
| Cycle | Amino acid | Solvent for couplings: DMF |
| 1 | Fmoc-Gly-OH | 3.0 AA/6.0 DIEA |
| | | 4 h, rt. |
| 2 | Fmoc-L-Glu(tBu)-OH | 2 × 30 min De-Fmoc cycles, |
| | | 6 × 2 min post-dep washes, |
| | | 3.0 AA/3.3 DIC/3.0 Oxyma |
| | | 6 h, rt. |
| 3 | Fmoc-L-Leu-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 4 | Fmoc-L-Leu-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 5 | Fmoc-L-Tyr(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 6 | Fmoc-L-Glu(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 7 | Fmoc-L-Ile-OH | 2 × 30 min De-Fmoc cycles, |
| | | 6 × 2 min post-dep washes, |
| | | 3.0 AA/3.3 DIC/3.0 Oxyma |
| | | 6 h, rt. |
| 8 | Fmoc-L-Phe-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 9 | Fmoc-Ala-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 10 | Fmoc-Aib-OH | 2 × 30 min De-Fmoc cycles, |
| | | 6 × 2 min post-dep washes, |
| | | 3.0 AA/3.3 DIC/3.0 Oxyma |
| | | 12 h, rt. |
| 11 | Fmoc-L-Gln(Trt)-OH | 3 × 30 min De-Fmoc cycles, |
| | | 6 × 2 min post-dep washes, |
| | | 3.0 AA/3.3 DIC/3.0 Oxyma |
| | | 18 h, rt. |
| 12 | Fmoc-L-Ala-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 13 | Fmoc-L-Lys(t- | 3 × 30 min De-Fmoc cycles, |
| | BuOOC—(CH₂)₁₈—COO- | 6 × 2 min post-dep washes, |
| | γ-L-Glu-AEEA) | 2.0 AA/4.0 DIEA/2.0 PyBOP |
| | | 16 h, rt. |
| 14 | Fmoc-L-Lys(Boc)-OH | 2 × 30 min De-Fmoc cycles, |
| | | 6 × 2 min post-dep washes, |
| | | 3.0 AA/3.3 DIC/3.0 Oxyma |
| | | 8 h, rt. |
| 15 | Fmoc-L-Asp(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt. |

### SEQ ID NO:45 Soft Cleavage:

1) Add resin bound SEQ ID NO:45 (4.0 g, ˜1.34 mmol) and charge 40 mL cleavage cocktail TFA/DCM (1/99, v/v/v). 2) Stir it for 10 min at rt. 3) Filter and collect the filtrate. 4) Neutralize the filtrate with 0.44 mL pyridine (1/1, mol/mol). 5) Repeat Steps 1-4 for 3 more times. 6) Concentrate the combined filtrate to dryness. 7) Dissolve the slurry with 10 mL of DMSO. 8) Charge the DMSO solution slowly to cold 100 mL water with stirring. 9) Filter and collect precipitation. 10) Reslurry with 50 mL water for 2 times. 11) Dry in vacuum overnight to produce SEQ ID NO:45 (2.5 g, 58% yield) of a white solid. Analysis of the isolated solid using UPLC (95.0 area %). LC-MS ([M+2H]2⁺/2): 1604.97.

### Example 53: Alternative Synthesis of SEQ ID NO:10

TABLE 32
The synthesis uses Fmoc-Leu-OH 2-CTC resin with
a loading of 0.80 mmol/g. The general SPPS procedure
is used with the following modifications:

| | | SPPS conditions |
|---|---|---|
| Cycle | Amino acid | Solvent for couplings: DMF |
| 1 | Fmoc-L-αMe-Leu-OH | 3.0 AA/6.0DIEA |
| | | 18 h, rt |
| 2 | Fmoc-L-Ile-OH | 3 × 30 min De-Fmoc cycles, |
| | | 6 × 2 min post-dep washes, |
| | | 3.0 AA/3.3 DIC/3.0 Oxyma |
| | | 18 h, rt. |
| 3 | Fmoc-L-Ser(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 4 | Fmoc-L-Tyr(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt |
| 5 | Fmoc-L-Asp(tBu)-OH | 2 × 30 min De-Fmoc cycles, |
| | | 6 × 2 min post-dep washes, |
| | | 3.0 AA/3.3 DIC/3.0 Oxyma |
| | | 4h, rt. |
| 6 | Fmoc-L-Ser(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 7 | Fmoc-L-Thr(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 8 | Fmoc-L-Phe-OH | 2 × 30 min De-Fmoc cycles, |
| | | 6 × 2 min post-dep washes, |
| | | 3.0 AA/3.3 DIC/3.0 Oxyma |
| | | 4 h, rt |
| 9 | Fmoc-L-Thr(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt |
| 10 | Fmoc-Gly-OH | 2 × 30 min De-Fmoc cycles, |
| | | 6 × 2 min post-dep washes, |
| | | 3.0 AA/3.3 DIC/3.0 Oxyma |
| | | 6 h, rt. |
| 11 | Fmoc-L-Gln(Trt)-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 12 | Fmoc-Aib-OH | 2 × 30 min De-Fmoc cycles, |
| | | 6 × 2 min post-dep washes, |
| | | 3.0 AA/3.3 DIC/3.0 Oxyma |
| | | 8 h, rt. |
| 13 | Boc-L-Tyr(tBu)-OH | 2 × 30 min De-Fmoc cycles, |
| | | 6 × 2 min post-dep washes, |
| | | 3.0 AA/3.3 DIC/3.0 Oxyma |
| | | 16 h, rt. |

Soft cleavage: 1) Add resin bound SEQ ID NO:10 (60 g, ˜40 mmol) and charge 600 mL cleavage cocktail TFA/DCM (1/99, v/v/v). 2) Stir it for 10 min at rt. 3) Filter and collect the filtrate. 4) Neutralize the filtrate with 6.6 mL pyridine (1/1, mol/mol). 5) Repeat Steps 1-4 for 3 more times. 6) Concentrate the combined filtrate to dryness. 7) Dissolve the slurry with 60 mL of DMSO. 8) Charge the DMSO solution slowly to cold 600 mL water with stirring. 9) Filter and collect precipitation. 10) Reslurry with 300 mL water for 2 times. 11) Dry in vacuum overnight to produce SEQ ID NO:10 (56.4 g, 125% yield) as a wet solid. Analysis of the isolated solid using UPLC (93.1 area %). LC-MS ([M+H]⁺): 2341.52.

### Example 54: Synthesis of SEQ ID NO:46 (Compound 49) by LPPS

To a 20 mL glass scintillation vial, add SEQ ID NO:7 (250 mg, 77.9 μmol), SEQ ID NO:45 (103 mg, 90.7 μmol), and DMSO (5 mL). Add DIEA (81 μL, 0.47 mmol) to this solution followed by PyAOP (7-azabenzotriazol-1-yloxy)tripyrrolidinophosphonium hexafluorophosphate) (100 mg, 192 μmol). Stir the reaction for 4 hours then, slowly add 35 mL cold water. Collect the precipitated product by filtration and subsequently wash with water (2×35 mL). Dry the wetcake under vacuum to obtain SEQ ID NO:46 (Compound 49) as a white solid (199 mg, 61% yield). UPLC: 46.2 area %.

### Example 55: Synthesis of SEQ ID NO:47 (Compound 50) by LPPS

To a 20 mL glass scintillation vial, add SEQ ID NO:46 (500 mg, 120 μmol), followed by 2 mL MeCN (2 mL). Charge Et₂NH (0.5 mL, 4.8 mmol), and stir for 4 h. Concentrate the solution to dryness. Charge 5 mL MeCN and concentrate to dryness again. Repeat MeCN addition and drying for 2-3 more times. Charge 1 mL of MeCN to dissolve the slurry, then charge the reaction solution slowly to 15 mL cold MTBE with stirring. Filter and collect precipitation, and reslurry with 10 mL MTBE for 2 times. Dry the wetcake under vacuum to obtain SEQ ID NO:47 (Compound 50) as a white solid (200 mg, 42% yield). UPLC: 75.3 area %. LC-MS [M+3H]³⁺/3: 1331.60.

### Example 56: Synthesis of SEQ ID NO:48 (Compound 51) by LPPS

To a 20 mL glass scintillation vial, add SEQ ID NO:10 (75 mg, 32.1 μmol), SEQ ID NO:47 (100 mg, 25.0 μmol), 1-Hydroxy-7-azabenzotriazole (HOAt; 5 mg, 36.8 μmol), and DMSO (2 mL). Add DIEA (30 μL, 173 μmol) to this solution followed by PyAOP (33 mg, 63 μmol). Stir the reaction for 5 hours then, slowly add 15 mL cold water. Collect the precipitated product by filtration and subsequently wash with water (3×10 mL). Dry the wetcake under vacuum to obtain SEQ ID NO:48 (Compound 51) as a white solid (120 mg, 76% yield). UPLC: 53.6 area %.

### Example 57: Synthesis of SEQ ID NO:6 by Global Deprotection

Charge 1 mL cleavage cocktail solution TFA/H₂O/TIPS/DTT (0.925/0.025/0.025/0.025, v/v/v/v), then a sample of SEQ ID NO:48 (76 mg, 12.0 μmol) is added to this mixture to provide a solution. The mixture is stirred at about ambient temperature for about 3 hours. Pour the reaction mixture to −15° C. MTBE (10 mL), stir the resulting suspension for about 30 min. Perform filtration through filter and wash the wetcake with MTBE (2×10 mL). The wet cake is dried at 35° C. in vacuo resulting in SEQ ID NO:6 (80 mg, 44.8 area %, 140% crude yield) as a wet solid. UPLC: 44.8 area %. LC-MS [M+3H]³⁺/3: 1183.20.

### Example 58: Alterative Synthesis of SEQ ID NO:11

TABLE 33
The synthesis uses Fmoc-Gly-CTC resin with a loading
of 0.835 mmol/g. The general SPPS procedure is
used with the following modifications.

| | | SPPS conditions |
|---|---|---|
| Cycle | Amino acid | Solvent for couplings: DMF |
| 1 | Fmoc-L-Leu-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 2 | Fmoc-L-Leu-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 3 | Fmoc-L-Tyr(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 4 | Fmoc-L-Glu(tBu)-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 5 | Fmoc-L-Ile-OH | 2 × 30 min De-Fmoc cycles, |
| | | 6 × 2 min post-dep washes, |
| | | 3.0 AA/3.3 DIC/3.0 Oxyma |
| | | 4 h, rt. |
| 6 | Fmoc-L-Phe-OH | 3 × 30 min De-Fmoc cycles, |
| | | 6 × 2 min post-dep washes, |
| | | 3.0 AA/3.3 DIC/3.0 Oxyma |
| | | 4 h, rt. |
| 7 | Fmoc-Ala-OH | 3 × 30 min De-Fmoc cycles, |
| | | 6 × 2 min post-dep washes, |
| | | 3.0 AA/3.3 DIC/3.0 Oxyma |
| | | 4 h, rt. |

Soft cleavage: 1) Add resin bound SEQ ID NO:11 (10.0 g, ˜4.4 mmol) and charge 100 mL cleavage cocktail TFA/DCM (1/99, v/v). 2) Stir it for 10 min at rt. 3) Filter and collect the filtrate. 4) Neutralize the filtrate with 1.1 mL pyridine (1/1, mol/mol). 5) Repeat Steps 1-4 for 3 more times. 6) Concentrate the combined filtrate to dryness. 7) Dissolve the slurry with 20 mL of DMSO. 8) Charge the DMSO solution slowly to cold 100 mL water with stirring. 9) Filter and collect precipitation. 10) Re-slurry with 100 mL water for 2 times. 11) Dry in vacuum overnight to produce SEQ ID NO:11 (4.01 g, 62% yield) of a white solid. Analysis of the isolated solid using UPLC (97.6 area %). LC-MS [M+H]⁺: 1445.78.

### Example 59: Alternative Synthesis of SEQ ID NO:18

TABLE 34
The synthesis uses Fmoc-Sieber amide resin with
a loading of 0.71 mmol/g. The general SPPS procedure
is used with the following modifications:

| Cycle | Amino acid | Coupling reaction |
|---|---|---|
| 1 | Fmoc-L-Ser(t-Bu)-OH | 2 × 30 min De-Fmoc cycles, |
| | | 6 × 2 min post-dep washes, |
| | | DIC/oxyma/AA = |
| | | 3.3/3/3 4 hours coupling |
| 2 | Fmoc-L-Pro-OH | 2 × 30 min De-Fmoc cycles, |
| | | 6 × 2 min post-dep washes, |
| | | DIC/oxyma/AA = |
| | | 3.3/3/3 6 hours coupling |
| 3 | Fmoc-L-Pro-OH | 3 × 30 min De-Fmoc cycles, |
| | | 6 × 2 min post-dep washes, |
| | | DIC/oxyma/AA = |
| | | 3.3/3/3 12 hours coupling |
| 4 | Fmoc-L-Pro-OH | 3 × 30 min De-Fmoc cycles, |
| | | 6 × 2 min post-dep washes, |
| | | DIC/oxyma/AA = |
| | | 3.3/3/3 12 hours coupling |
| 5 | Fmoc-L-Ala-OH | 3 × 30 min De-Fmoc cycles, |
| | | 6 × 2 min post-dep washes, |
| | | DIC/oxyma/AA = |
| | | 3.3/3/3 10 hours coupling |
| 6 | Fmoc-Gly-OH | 2 × 30 min De-Fmoc cycles, |
| | | 6 × 2 min post-dep washes, |
| | | DIC/oxyma/AA = |
| | | 3.3/3/3 4 hours coupling |
| 7 | Fmoc-L-Ser(tBu)-OH | 2 × 30 min De-Fmoc cycles, |
| | | 6 × 2 min post-dep washes, |
| | | DIC/oxyma/AA = |
| | | 3.3/3/3 4 hours coupling |
| 8 | Fmoc-L-Ser(tBu)-OH | 2 × 30 min De-Fmoc cycles, |
| | | 6 × 2 min post-dep washes, |
| | | DIC/oxyma/AA = |
| | | 3.3/3/3 6 hours coupling |
| 9 | Fmoc-L-Pro-OH | 2 × 30 min De-Fmoc cycles, |
| | | 6 × 2 min post-dep washes, |
| | | DIC/oxyma/AA = |
| | | 3.3/3/3 6 hours coupling |
| 10 | Fmoc-Gly-OH | 3 × 30 min De-Fmoc cycles, |
| | | 6 × 2 min post-dep washes, |
| | | DIC/oxyma/AA = |
| | | 3.3/3/3 10 hours coupling |
| 11 | Fmoc-Gly-OH | 2 × 30 min De-Fmoc cycles, |
| | | 6 × 2 min post-dep washes, |
| | | DIC/oxyma/AA = |
| | | 3.3/3/3 4 hours coupling |
| 12 | Fmoc-L-Glu(OtBu)-OH | 2 × 30 min De-Fmoc cycles, |
| | | 6 × 2 min post-dep washes, |
| | | DIC/oxyma/AA = |
| | | 3.3/3/3 4 hours coupling |
| 13 | Fmoc-L-Leu-OH | 2 × 30 min De-Fmoc cycles, |
| | | 6 × 2 min post-dep washes, |
| | | DIC/oxyma/AA = |
| | | 3.3/3/3 6 hours coupling |
| 14 | Fmoc-L-Leu-OH | 2 × 30 min De-Fmoc cycles, |
| | | 6 × 2 min post-dep washes, |
| | | DIC/oxyma/AA = |
| | | 3.3/3/3 6 hours coupling |
| 15 | Fmoc-L-Tyr(tBu)-OH | 2 × 30 min De-Fmoc cycles, |
| | | 6 × 2 min post-dep washes, |
| | | DIC/oxyma/AA = |
| | | 3.3/3/3 4 hours coupling |
| 16 | Fmoc-L-Glu(OtBu)-OH | 2 × 30 min De-Fmoc cycles, |
| | | 6 × 2 min post-dep washes, |
| | | DIC/oxyma/AA = |
| | | 3.3/3/3 4 hours coupling |
| 17 | Fmoc-L-Ile-OH | 2 × 30 min De-Fmoc cycles, |
| | | 6 × 2 min post-dep washes, |
| | | DIC/oxyma/AA = |
| | | 3.3/3/3 6 hours coupling |
| 18 | Fmoc-L-Phe-OH | 2 × 30 min De-Fmoc cycles, |
| | | 6 × 2 min post-dep washes, |
| | | DIC/oxyma/AA = |
| | | 3.3/3/3 4 hours coupling |
| 19 | Fmoc-L-Ala-OH | 2 × 30 min De-Fmoc cycles, |
| | | 6 × 2 min post-dep washes, |
| | | DIC/oxyma/AA = |
| | | 3.3/3/3 4 hours coupling |

Soft cleavage: 1) After the final 2×30 min De-Fmoc cycles, charge the resin bound SEQ ID NO:18 (8.2 g, ˜ 3.1 mmol) to 40 mL cleavage cocktail TFA/HFIP/DCM (1/25/74, v/v/v), and stir it for 5 minutes at 25° C. 2) Filter and collect the filtrate and neutralize the filtrate with 0.44 mL pyridine (1/1, mol/mol). 3) Repeat the cleavage process for 2 more times. 4) Concentrate the combined filtrate to dryness. 5) Dissolve the slurry with 10 mL of DMSO and charge the DMSO solution slowly to 200 mL MTBE with stirring. 6) Filter and collect precipitation. 7) Re-slurry with 2 more time with 40 mL MTBE and filter the precipitate. 8) Dry the crude material in vacuum overnight to obtain 2.76 grams of crude (41.4% yield) with 92.5% purity by HPLC. LC-MS [M+2H]²⁺/2: 1113.90.

### Example 60: Alternative Synthesis of SEQ ID NO:20

TABLE 35
The synthesis uses 2-CTC resin with a loading of 0.80 mmol/g. The
general SPPS procedure is used with the following modifications.

| | | SPPS conditions |
|---|---|---|
| Cycle | Amino acid | Solvent for couplings: DMF |
| 1 | Fmoc-Aib-OH | 3.0 AA/6.0 DIEA |
| | | 4 h, rt. |
| | | Capping performed using 10 |
| | | vol MeOH/DIEA/DMF, 5/15/80, |
| | | v/v/v at rt |
| 2 | Fmoc-L-Gln(Trt)-OH | 3 × 30 min De-Fmoc cycles, |
| | | 6 × 2 min post-dep washes, |
| | | 3.0 AA/3.3 DIC/3.0 |
| | | Oxyma 18 h, rt. |
| 3 | Fmoc-Ala-OH | 2 × 30 min De-Fmoc cycles, |
| | | 6 × 2 min post-dep washes, |
| | | 3.0 AA/3.3 DIC/3.0 |
| | | Oxyma 4 h, rt |
| 4 | Fmoc-L-Lys(t- | 3 × 30 min De-Fmoc cycles, |
| | BuOOC—(CH₂)₁₈—COO- | PyBOP/DIEA/AA = |
| | γ-L-Glu-AEEA) | 2/4/2 >16 hours coupling |
| 5 | Fmoc-Lys(Boc)-OH | 2 × 30 min De-Fmoc cycles, |
| | | 3.0 AA/3.3 DIC/3.0 |
| | | Oxyma >8 hours coupling |
| 6 | Fmoc-Asp(OtBu)-OH | 2 × 30 min De-Fmoc cycles, |
| | | 3.0 AA/3.3 DIC/3.0 |
| | | Oxyma >4 hours coupling |
| 7 | Fmoc-Leu-OH | 2 × 30 min De-Fmoc cycles, |
| | | 3.0 AA/3.3 DIC/3.0 |
| | | Oxyma >4 hours coupling |
| 8 | Fmoc-L-αMe-Leu-OH | 3 × 30 min De-Fmoc cycles, |
| | | 6 × 2 min post-dep washes, |
| | | 3.0 AA/3.3 DIC/3.0 |
| | | Oxyma >12 h, rt. |
| 9 | Fmoc-L-Ile-OH | 3 × 30 min De-Fmoc cycles, |
| | | 6 × 2 min post-dep washes, |
| | | 3.0 AA/3.3 DIC/3.0 |
| | | Oxyma >18 h, rt. |
| 10 | Fmoc-L-Ser(tBu)-OH | 2 × 30 min De-Fmoc cycles, |
| | | 6 × 2 min post-dep washes, |
| | | 3.0 AA/3.3 DIC/3.0 |
| | | Oxyma >4 h, rt. |
| 11 | Fmoc-L-Tyr(tBu)-OH | 2 × 30 min De-Fmoc cycles, |
| | | 6 × 2 min post-dep washes, |
| | | 3.0 AA/3.3 DIC/3.0 |
| | | Oxyma >4 h, rt |
| 12 | Fmoc-L-Asp(tBu)-OH | 2 × 30 min De-Fmoc cycles, |
| | | 6 × 2 min post-dep washes, |
| | | 3.0 AA/3.3 DIC/3.0 |
| | | Oxyma >4 h, rt. |
| 13 | Fmoc-L-Ser(tBu)-OH | 2 × 30 min De-Fmoc cycles, |
| | | 6 × 2 min post-dep washes, |
| | | 3.0 AA/3.3 DIC/3.0 |
| | | Oxyma >4 h, rt. |
| 14 | Fmoc-L-Thr(tBu)-OH | 2 × 30 min De-Fmoc cycles, |
| | | 6 × 2 min post-dep washes, |
| | | 3.0 AA/3.3 DIC/3.0 |
| | | Oxyma >4 h, rt. |
| 15 | Fmoc-L-Phe-OH | 2 × 30 min De-Fmoc cycles, |
| | | 6 × 2 min post-dep washes, |
| | | 3.0 AA/3.3 DIC/3.0 |
| | | Oxyma >4 h, rt |
| 16 | Fmoc-L-Thr(tBu)-OH | 2 × 30 min De-Fmoc cycles, |
| | | 6 × 2 min post-dep washes, |
| | | 3.0 AA/3.3 DIC/3.0 |
| | | Oxyma >4 h, rt |
| 17 | Fmoc-Gly-OH | 2 × 30 min De-Fmoc cycles, |
| | | 6 × 2 min post-dep washes, |
| | | 3.0 AA/3.3 DIC/3.0 |
| | | Oxyma >4 h, rt. |
| 18 | Fmoc-L-Gln(Trt)-OH | 2 × 30 min De-Fmoc cycles, |
| | | 6 × 2 min post-dep washes, |
| | | 3.0 AA/3.3 DIC/3.0 |
| | | Oxyma >4 h, rt. |
| 19 | Fmoc-Aib-OH | 2 × 30 min De-Fmoc cycles, |
| | | 6 × 2 min post-dep washes, |
| | | 3.0 AA/3.3 DIC/3.0 |
| | | Oxyma >8 h, rt. |
| 20 | Boc-L-Tyr(tBu)-OH | 2 × 30 min De-Fmoc cycles, |
| | | 6 × 2 min post-dep washes, |
| | | 3.0 AA/3.3 DIC/3.0 |
| | | Oxyma >16 h, rt. |

Soft cleavage: 1) Add resin bound SEQ ID NO:20 (5.7 g, ˜10 mmol) and charge 60 mL cleavage cocktail TFA/DCM (1/99, v/v/v). 2) Stir it for 10 min at rt. 3) Filter and collect the filtrate. 4) Neutralize the filtrate with 6.6 mL pyridine (1/1, mol/mol). 5) Repeat Steps 1-4 for 3 more times. 6) Concentrate the combined filtrate to dryness. 7) Dissolve the slurry with 30 mL of DMSO. 8) Charge the DMSO solution slowly to cold 300 mL water with stirring. 9) Filter and collect precipitation. 10) Re-slurry with 200 mL water for 2 times. 11) Dry in vacuum overnight to produce SEQ ID NO:20 (4.5 g, 63.4% yield) of a white solid. Analysis of the isolated solid using UPLC (99.4 area %). LC-MS [M+2H]²⁺/2: 2053.39.

### Example 61: Synthesis of SEQ ID NO:49 (Compound 52) by LPPS

To a 20 mL glass scintillation vial, charge SEQ ID NO:11 (1.0 eq, 145 mg), SEQ ID NO:7 (1.1 eq, 125 mg), and DMSO/MeCN (5 mL, 4/1, v/v) to dissolve all material. Add DIEA (3.0 eq, 0.055 mL) to the reaction mix followed by PyOxim (1.5 eq, 80 mg). Stir the reaction for 4 hours then, slowly add 40 ml of water with stirring. Collect the precipitated product by filtration and subsequently wash with water (2×40 mL). Dry the wet cake under vacuum to obtain crude solid SEQ ID NO:49 as a white solid (180 mg, 73.2% yield) with 89.5% purity by HPLC. LC-MS [M+2H]²⁺/2: 1225.2.

### Example 62: Synthesis of SEQ ID NO:18 by LPPS

To a 20 mL glass scintillation vial, add SEQ ID NO:49 (700 mg), followed by 8 mL DMSO (2 mL). Charge Et2NH (2.0 mL), and stir for 4 h. Concentrate the solution to dryness. Charge 60 mL cold MTBE with stirring. Filter and collect precipitation, and reslurry with 60 mL MTBE for 2 times. Dry the wetcake under vacuum to obtain SEQ ID NO:18 as a white solid (520 mg, 82% yield) with 82.3% purity by HPLC. LC-MS [M+2H]²⁺/2: 1113.8.

### Example 63: Synthesis of SEQ ID NO:48 by LPPS

To a 20 mL glass scintillation vial, add SEQ ID NO:20 (1.0 eq, 84 mg), SEQ ID NO:18 (1.1 eq, 50 mg), 1-Hydroxy-7-azabenzotriazole (HOAt; 1.0 eq, 3 mg), and DMSO (2 mL) to dissolve all materials. Add DIEA (6 eq, 21 μL) to this solution followed by PyAOP (2.5 eq, 22 mg) and mix for 6 hours. Charge additional PyAOP (1.0 eq, 9 mg) and DIEA (2.5 eq, 9 μL) and mix for 12 hours. Charge additional PyAOP (1.0 eq, 9 mg) and DIEA (2.5 eq, 9 μL) and mix for 6 hours. Charge the reaction solution slowly to cold water with stirring. Collect the precipitated product by filtration and subsequently wash with water 3 times (3×10 ml). Dry the product under vacuum to obtain white solid SEQ ID NO:48 (90 mg, 69.8% yield). UPLC: 81.9 area %.

### Example 64: Global Deprotection of SEQ ID NO:48 to Produce SEQ ID NO:6

Global deprotection is carried out using the following procedure: 1) Charge 4 mL cleavage cocktail TFA/H₂O/TIPS/DTT (0.925/0.025/0.025/0.025) into R1, followed by charging of SEQ ID NO:48 (180 mg). 2) Stir it for 3 hours at 20-30° C. 3) Pour the solution to chilled MTBE (30 mL). Stir the suspension for 0.5 hours. 4) Perform filtration through filter followed by MTBE washing (30 mL) twice. 5) Dry the wet cake under reduce pressure until constant weight. 6) Obtain 180 mg of dried crude obtained with 66.3% purity by HPLC.

### Example 65: Native Chemical Ligation

Synthesis of Resin Compound 53:

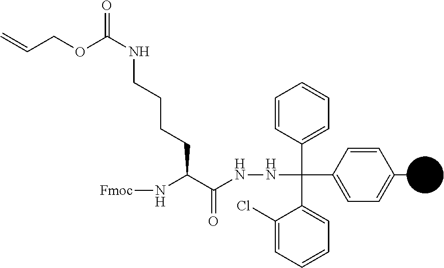

Fmoc-hydrazine-2-chlorotrityl resin (30.06 g, 25.5 mmol) is swollen in 300 mL DCM for 15 min. It is swollen with 2×400 mL DMF for 15 min each. Fmoc deprotection is performed with 3×400 mL 20% piperidine/DMF for 30 min each. The resin is then washed with 5×400 mL DMF. Fmoc-L-Lys(alloc)-OH (34.63 g, 76.5 mmol, 3.0 equiv) and HBTU (29.17 g, 76.9 mmol, 3.02 equiv) are dissolved in 400 mL of DMF. N, N-diisopropylethylamine (27 mL, 155 mmol, 6.08 equiv) is added to the amino acid solution. The solution is then added to the resin preparation XX and is allowed to stir for 6 hours. The resin is washed with 5×400 mL DMF, then 5×300 mL DCM and the resin is dried at 35° C. in a vacuum oven for about 16 hours. The resin loading is determined to be 0.52 mmol/g by quantitative NMR.

### Example 66: Synthesis of Peptide Hydrazide SEQ ID NO:50 (Compound 54)

About 12.19 g of resin Compound 53 (5.4 mmol, loading value: 0.44 mmol/g) is swollen with 3×120 mL DMF for 15 min each. Peptide hydrazide (SEQ ID NO:50 is synthesized using standard SPPS as previously described.

Deprotection: 4×100 mL of 20% v/v piperidine in DMF, 20 minutes each.
Couplings: 3 equivalents of amino acid, 3 equivalents of OXYMA and 3.3 equivalents of DIC are used for amino acid coupling.
During the SPPS, the resin is washed with 5×120 mL DMF with 5 min N₂ mix after each coupling and the final iteration of fmoc deprotection.
At the end of the peptide hydrazide synthesis, the resin is washed with DCM with N₂ mixing. The resin is dried on the peptide synthesizer.

### Alloc Deprotection and Sidechain Coupling:

The resin is washed with 5×120 mL DCM with 5 min stir. A solution of palladium tetrakis (500 mg, 0.43 mmol, 0.1 equiv) and phenylsilane (0.7 mL, 5.7 mmol, 1.02 equiv) is made in 75 mL DCM. It is added to the resin and stirred for 20 min. It is washed with 5×120 mL DCM and stirred for 5 min each. The alloc deprotection with Pd(PPh₃)₄ and PhSiH₃ is repeated twice.

The resin is washed with 5×120 mL DMF and stirred for 5 min each. TNTU (3.95 g, 10.82 mmol, 2 equiv) is dissolved in the DMF solution of (S)-13-(tert-butoxycarbonyl)-36,36-dimethyl-10,15,34-trioxo-3,6,35-trioxa-9,14-diazaheptatriacontanoic acid (Compound 25, 27 mL, 0.29 g/mL, 7.873 g, 10.8 mmol, 2 equiv) and this solution is made up to 75 mL with DMF. 3.8 mL of N, N-diisopropylethylamine (3.8 mL, 21.82 mmol, 4.0 mmol) is added to the solution of Compound 25 and stirred for 10 min. It is then added to the resin and stirred for 14 hours. The resin is then washed with 5×120 mL DMF (5 min stir) and 5×120 mL DCM (5 min stir). The resin is dried in a vacuum oven for about 16 hours at 35° C.

### Global Deprotection and Cleavage:

250 mL of the cleavage cocktail made with 2.5% w/v dithiothreitol (DTT), 2.5% v/v water, 2.5% v/v triisopropylsilane (TIPS) and 92.5% trifluoroacetic acid (TFA) is added to the dried resin (22.2 g) in a 500 mL three-necked round bottom flask and stirred for about 2.5 hours. The resin is filtered and washed with 2×7.5 mL TFA. The filtrate is poured into 1.40 L cold MTBE and the peptide precipitated out immediately. The filtration flask is washed with 2×5 mL TFA and poured into the cold MTBE. It is cooled down to −20° C. for half an hour and then centrifuged. The peptide precipitate is then washed twice with 300 mL MTBE and centrifuged. The peptide precipitate is dried in a vacuum oven at 27° C. for about 16 hours. About 9.9 g of the crude SEQ ID NO:50 is obtained after drying.

### Example 67: Synthesis of the Thioester SEQ ID NO:51 (Compound 55)

Crude peptide hydrazide (SEQ ID NO:50, 3.65 g, 1.41 mmol) is dissolved in 250 mL of the ligation buffer (6 M guanidine hydrochloride and 0.1 M sodium hydrogen phosphate monobasic, pH about 7.0). The pH is adjusted to about 3.3 with 5 N HCl solution and the solution is cooled to −15° C. in an acetone-ice bath. 2.5 mL of 4.31 M sodium nitrite solution (742.7 mg, 10.8 mmol, 7.6 equiv.) is added to the peptide hydrazide solution and allowed to stir for 15 min at −15° C. Meanwhile, 4-mercaptophenol (1.052 g, 8.34 mmol) is suspended in 3 mL of the ligation buffer, pH is adjusted to about 7.0 with 5 N NaOH solution and made up to 10 mL with ligation buffer (6 M guanidine hydrochloride and 0.1 M sodium hydrogen phosphate monobasic, pH about 7.0). After 15 min, 7.5 mL of the 4-mercaptophenol is added to the peptide hydrazide solution to cause in-situ thiolysis of the peptidyl azide generated from SEQ ID NO:50.

The pH of the reaction mixture is adjusted to about 6.5 with 5 N sodium hydroxide solution. Thiolysis of the peptidyl azide is allowed to run for 15 min and the crude thioester mixture is purified by RP-HPLC on a Phenomenex Luna C18 10 μm column (30 mm×250 mm) at ambient temperature with a linear gradient of 30-55% acetonitrile in water over 25 min after 10% acetonitrile in water for the first 3 min and 10-30% acetonitrile in water from 3 min to 5 min with constant 5% ammonium acetate throughout the purification. This yields about 0.415 g of the peptide thioester (SEQ ID NO:51) [Expected (mass+2H⁺)/2=1342.7052, observed (mass+2H⁺)/2=1342.6958].

### Example 68: Native Chemical Ligation to Synthesize SEQ ID NO:53

Aqueous solution of 6 M guanidine hydrochloride and 0.1 M sodium hydrogen phosphate monobasic (pH about 7.0) is the ligation buffer used in native chemical ligation. The buffer is degassed with nitrogen gas for 15 min. 4-mercaptophenol (193 mg, 1.5 mmol, 10 equiv), tris(2-carboxyethyl)phosphine (TCEP, 656.6 mg, 2.3 mmol, 15.3 equiv) and ascorbic acid (269 mg, 1.5 mmol, 10 equiv) are taken in a 3 neck-round bottom flask. The flask is under nitrogen gas. 41 mL of the ligation buffer is added to dissolve the reagents in the round bottom flask. The pH of the solution is adjusted to about 7.0 with 5 N NaOH solution. The peptide thioester SEQ ID NO:51 ((406.8 mg, 0.15 mmol) and the N-terminal cysteine fragment SEQ ID NO 52 (Compound 56) (326.5 mg, 0.15 mmol, 1 equiv) are added to the above solution. pH is adjusted to about 7.0 with 5 N NaOH solution. The reaction mixture is allowed to stir for about 10 hours under nitrogen. Most of the thioester SEQ ID NO:51 is consumed and hence, the reaction mixture is stored in a freezer at −20° C. for about 14 hours. SEQ ID NO:53 is purified by RP-HPLC on a Phenomenex Luna C18 10 μm column (30 mm×250 mm) at ambient temperature with a linear gradient of 30-50% acetonitrile in water over 25 min after 20% acetonitrile in water for the first 3 min and 20-30% acetonitrile in water from 3 min to 5 min with constant 5% ammonium acetate throughout the purification. This yields about 0.52 g of the cysteine analogue SEQ ID NO:53 [Expected (mass+3H⁺)/3=1587.8219, observed (mass+3H⁺)/3=1587.8198].

Photodesulfurization: Aqueous buffer of 3 M guanidine hydrochloride and 0.1 M sodium hydrogen phosphate monobasic (pH about 7.0) is freshly made. 0.5 mL solution of 7.64 mM tris(2,2′-bipyridyl)dichlororuthenium(II) hexahydrate (2.86 mg, 0.004 mmol) is made in the buffer. Tris(2-carboxyethyl)phosphine (TCEP, 64.3 mg, 0.22 mmol) is suspended in the buffer and the pH is adjusted to about 7.0 with 5 N NaOH solution. This is diluted to 2 mL with the buffer. SEQ ID NO:53 (10 mg, 0.0021 mmol) is dissolved in 4 mL of the buffer in a 7 mL scintillation vial. Triphenylphosphine-3,3′,3″-trisulfonic acid trisodium salt (TPPTS, 231.2 mg, 0.41 mmol, 194 equiv) and 2-mercaptoethanesulfonic acid sodium salt (MESNa, 32.6 mg, 0.20 mmol, 95 equiv) are added to the solution of SEQ ID NO:6. 28 μL of tris(2,2′-bipyridyl)dichlororuthenium(II) hexahydrate (0.00021 mmol, 0.1 equiv) and 20 μL of TCEP solution (0.0022 mol, 1.0 equiv) are added to the reaction mixture. The vial Is placed in a Penn Optical Coatings photoreactor ml and stirred at 459 RPM with 91% LED intensity. Fan is run at 3564 RPM to prevent heating of the reaction mixture. After about 3.5 hours, TCEP (0.40 mg, 0.7 equiv) is further added and the reaction mixture is stirred in the photoreactor for 16 hours. After 16 hours, the reaction is complete and more than 95% of the SEQ ID NO:53 is converted into SEQ ID NO:6.

Metal-free desulfurization: Aqueous solution of 6 M guanidine hydrochloride and 0.1 M sodium hydrogen phosphate monobasic (pH 7.0) is the buffer used for this reaction. The buffer is thoroughly purged with nitrogen gas for more than an hour. SEQ ID NO:53 (40.3 mg, 0.0085 mmol), 2,2′-azobis[2-(2-imidazolin-2-yl)propane]dihydrochloride (27.7 mg, 0.0857 mmol, 10.1 equiv), L-glutathione reduced (L-GSH, 25.9 mg, 0.0843 mmol, 10 equiv) and tris(2-carboxyethyl)phosphine (TCEP, 36.5 mg, 0.1273 mmol, 15.0 equiv) are dissolved in 4 mL of the buffer. The reaction mixture is degassed again with nitrogen gas for about 2 min and the pH is adjusted to about 7.0 with 5 N NaOH. The solution is stirred under nitrogen at 45° C. for 12 hours and then at the room temperature for 8 hours. After 20 hours, most of the SEQ ID NO:53 is converted to the SEQ ID NO:6. [Expected (mass+3H⁺)/3=1604.5153, observed (mass+3H⁺)/3=1577.1581].

### Example 69: Synthesis of SEQ ID NO:53 (Compound 57) by Native Chemical Ligation (NCL) Using SEQ ID NOs 52 and 54 Using Cysteinylprolyl Ester (CPE)

3M Buffer solution: Guanidine hydrochloride (2.86 g, 30.0 mmol), sodium dihydrogen phosphate (0.24 g, 2.0 mmol) and tris(2-carboxyethyl)phosphine hydrochloride [TCEP] (0.0166 g, 0.0579 mmol) is weighed into a 15 mL centrifuge tube, dissolved in deionized water and made up to approximately 9.5 mL. The pH of the buffer solution is adjusted to ˜8.3 by adding 5N NaOH as required. If the pH is overshot, it is readjusted to ˜8.3 by addition of 1N HCl.

### General Procedure for Ligation:

1 mL of the buffer solution is added to a 5 mL scintillation vial containing pre-weighed SEQ ID NO:54 [CPE-peptide analogue] (0.01 g, 0.003 mmol, 94.69 mass %) and SEQ ID NO:52 [Cys-peptide] (0.0073 g, 0.0032 mmol, 96.9 mass %). The peptide fragments are fully dissolved in the 3M buffer solution by sonication. The pH of the solution is recorded and readjusted by addition of 5N NaOH to pH-8.30. If the pH is overshot, it is readjusted to pH-8.3 by addition of 1N HCl. The solution is then transferred to a HPLC vial and the sample monitored at different time points by ELTIVO at 37° C. (32° C. internal temperature). Complete conversion to SEQ ID NO:53 analogue (up to 69% by Q-Tof) is generally observed after 18 h.

5M Buffer solution: Guanidine hydrochloride (4.78 g, 50.0 mmol), sodium dihydrogen phosphate (0.24 g, 2.0 mmol) and (tris(2-carboxyethyl)phosphine hydrochloride [TCEP] (0.0166 g, 0.0579 mmol) is weighed into a 15 mL centrifuge tube, dissolved in deionized water and made up to approximately 9.5 mL. The pH of the buffer solution is adjusted to ˜8.3 by adding 5N NaOH as required. If the pH is overshot, it is readjusted to ˜8.3 by addition of 1N HCl.

### General Procedure for Ligation:

3 mL of the buffer solution is added to a 5 mL scintillation vial containing pre-weighed SEQ ID NO:54 [CPE-peptide analogue] (0.01 g, 0.003 mmol, 94.69 mass %) and SEQ ID NO:52 [Cys-peptide] (0.0073 g, 0.0032 mmol, 96.9 mass %). The peptide fragments are fully dissolved in the 5M buffer solution by sonication. The pH of the solution is recorded and readjusted by addition of 5N NaOH to pH-8.30. If the pH is overshot, it is readjusted to pH-8.3 by addition of 1N HCl. The thiol [such as; MeSNa, thiophenol, hydroxythiophenol] (5 equiv.) is then added to the reaction solution. The pH is monitored again and further adjusted to pH-8.3 using 1N NaOH or 1N HCl as required. The solution is then transferred to a HPLC vial and the sample monitored at different time points HPLC at 37° C. (32° C. internal temperature). Complete conversion to SEQ ID NO:53 is generally observed after approximately 17 h (by HPLC).

### Example 70: Synthesis of Fmoc-L-Pro-glycolic Acid-L-Val-OH (Compound 58)

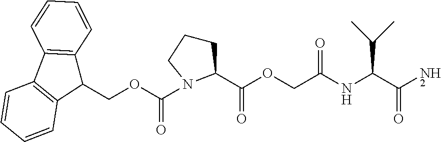 [SMILES: CC(C)[C@H](NC(=O)COC(=O)[C@@H]1CCCN1C(=O)OCC1c2ccccc2-c2ccccc21)C(N)=O]

### Step 1 (Fmoc-L-Val-OH Coupling):

Prior to the first coupling, Fmoc-Rink amide AM resin (0.74 g/mmol, 1.35 g, 1.00 mmol) is charged to the reaction vessel. The resin is swelled with 3×10 ml of DMF for 15 minutes each, then deprotected with 3×10 ml of 20% piperidine/DMF (v/v) for 30 minutes each and washed with 5×10 ml of DMF for 1 minute each. A solution is prepared of (2S)-2-(9H-fluoren-9-ylmethoxycarbonylamino)-3-methyl-butanoic acid (1.018 g, 3.00 mmol) and 1-hydroxybenzotriazole hydrate (0.74 g, 3.30 mmol, 60 mass %) in 10 ml of DMF. To this solution is added N, N′-diisopropylcarbodiimide (0.52 mL, 3.30 mmol) and the corresponding solution is added to the reaction vessel containing the swelled resin. The reaction is mixed for 1 hour at ambient temperature, then the liquid is drained. The resin is washed with 5×10 ml of DMF for 1 minute each and then forward processed to step 2.

### Step 2 (Glycolic Acid Coupling):

The Fmoc group is removed by treatment of the resin from step 1 with 3×10 ml of 20% piperidine/DMF (v/v) for 30 minutes each and washed with 5×10 ml of DMF for 1 minute each. A solution is prepared of glycolic acid (228 mg, 3.00 mmol) and 1-hydroxybenzotriazole hydrate (353 mg, 2.31 mmol) in 10 ml of DMF. To this solution is added N, N′-diisopropylcarbodiimide (0.52 mL, 3.30 mmol) and the corresponding solution is added to each reactor. The reaction is mixed for 5 hours at ambient temperature, then the liquid is drained. The resin is washed with 5×10 ml of DMF for 1 minutes and then forward processed to step 3.

### Step 3 (Fmoc-L-Pro-OH Coupling):

A solution is prepared of (2R)-1-(9H-fluoren-9-ylmethoxycarbonyl)pyrrolidine-2-carboxylic acid (1.012 g, 3.00 mmol), (2-(1H-benzotriazol-1-yl)-1,1,3,3-tetramethyluronium hexafluorophosphate (HBTU) (1.25 g, 3.30 mmol), and N,N-diisopropylethylamine (0.87 mL, 5.00 mmol) in 10 ml of DMF. The solution is shaken for a few minutes, then transferred to the reaction vessel containing the resin. The reaction is mixed for 16 hours at ambient temperature, then the liquid is drained. The resin is washed with 5×10 ml of DMF for 1 minutes and 5×10 ml of dichloromethane for 2 minutes, then dried to constant weight to provide 1.719 g of the title compound on resin.

A 50 mg sample of the peptide is cleaved from the resin with 2.0 mL of a solution consisting of 92.5% TFA, 2.5% triisopropylsilane, 2.5% water, and 2.5% dithiothreitol (v/v/v/w). The mixture is agitated on a rotary mixer for 1.5 hours, diluted with 16 ml of 80:20 DMSO/acetonitrile (v/v), and filtered to remove the resin. The filtrate is analyzed by LC/MS and shown to contain 75.5 area % desired tripeptide and 16.8% of product containing multiple glycolic acid additions.

### Example 71: Alternate Synthesis of Fmoc-L-Pro-Glycolic Acid-L-Val-OH

### Step 1 (Fmoc-L-Val-OH Coupling):

Prior to the first coupling, Rink amide AM resin (0.74 g/mmol, 1.35 g, 1.00 mmol) is charged to the reactor vessel. Each resin is swelled with 3×10 ml of DMF for 20 minutes each, then deprotected with 3×10 ml of 20% piperidine/DMF (v/v) for 30 minutes each and washed with 5×10 ml of DMF for 1 minute each. A solution is prepared of (2S)-2-(9H-fluoren-9-ylmethoxycarbonylamino)-3-methyl-butanoic acid (1.018 g, 3.00 mmol) and 1-hydroxybenzotriazole hydrate (353 mg, 2.31 mmol) in 10 ml of DMF. To this solution is added N, N′-diisopropylcarbodiimide (517 μL, 3.30 mmol) and the corresponding solution is added to the reaction vessel containing the swelled resin. The reaction is mixed for 4 hours at ambient temperature and then drained. The resin is washed with 5×10 ml of DMF for 1 minute each and forward processed to the next step.

### Step 2 (Fmoc-Glycolic Acid Coupling):

The Fmoc group is removed by treatment with 3×10 ml of 20% piperidine/DMF (v/v) for 30 minutes each and washed with 5×10 ml of DMF for 1 minute each. A solution is prepared of 2-(9H-fluoren-9-ylmethoxycarbonyloxy)acetic acid (894.9 mg, 3.00 mmol) and 1-hydroxybenzotriazole hydrate (353 mg, 2.31 mmol) in 10 ml of DMF. To this solution is added N, N′-diisopropylcarbodiimide (517 μL, 3.30 mmol) and the corresponding solution is added to the reactor containing the resin. The reaction is mixed for 16 hours at ambient temperature and the liquid is drained. The resin is washed with 5×10 ml of DMF for 1 minute each and then forward processed to the next step.

### Step 3 (Fmoc-L-Pro-OH Coupling):

The Fmoc group is removed by treatment with 3×10 ml of 20% piperidine/DMF (v/v) for 30 minutes each and washed with 5×10 ml of DMF for 1 minute each. A solution is prepared of (2S)-1-(9H-fluoren-9-ylmethoxycarbonyl)pyrrolidine-2-carboxylic acid (1.012 g, 3.00 mmol), (2-(1H-benzotriazol-1-yl)-1,1,3,3-tetramethyluronium hexafluorophosphate (HBTU) (1.25 g, 3.30 mmol), and N,N-diisopropylethylamine (870 μL, 5.00 mmol) in 10 ml of DMF. The corresponding solution is added to the reactor containing the resin. The reaction is mixed for 8 hours at ambient temperature and then drained. The resin is washed with 5×10 ml of DMF for 1 minute each and 5×10 ml of dichloromethane for 1 minute, then dried to constant weight to provide 1.622 g of the title compound on resin.

A 50 mg sample of the peptide is cleaved from the resin with 2.0 mL of a solution consisting of 92.5% TFA, 2.5% triisopropylsilane, 2.5% water, and 2.5% dithiothreitol (v/v/v/w). The mixture is agitated on a rotary mixer for 1.5 hours, diluted with 16 ml of 80:20 DMSO/acetonitrile (v/v), and filtered to remove the resin. The filtrate is analyzed by LC/MS and shown to contain 84.93 area % desired tripeptide no detectable multiple glycolic acid additions.

### Example 72: Synthesis of 2-(9H-fluoren-9-ylmethoxycarbonyloxy)acetic acid (Fmoc-Glycolic Acid) (Compound 59)

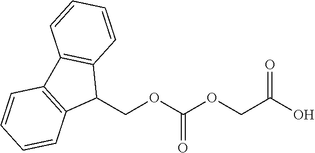 [SMILES: O=C(O)COC(=O)OCC1c2ccccc2-c2ccccc21]

Step 1 (tert-butyl 2-(9H-fluoren-9-ylmethoxycarbonyloxy)acetate):

To a magnetically stirred solution of tert-butyl 2-hydroxyacetate (10.00 g, 71.90 mmol, 95 mass %) in 120 ml of dichloromethane in a 500 mL round bottomed flask is added pyridine (60 mL, 742.0 mmol) in one portion. The resulting solution is cooled to 0-5° C. in an ice bath. To this solution is added a solution of 9-fluorenylmethyl chloroformate (20.00 g, 77.30 mmol) in 60 ml of dichloromethane dropwise via dropping funnel over 30 minutes. By the time the addition is complete, a precipitate had formed in the reaction. The ice bath is removed and the reaction mixture is allowed to stir for 18 hours at ambient temperature. More precipitate formed during the additional stir time. The reaction mixture is concentrated under reduced pressure to a solid-oil residue to remove most of the pyridine and dichloromethane, and then re-dissolved in 200 ml of dichloromethane. The solution is washed with 2×100 ml of 1M aqueous sodium bisulfate solution followed by 2×100 ml of saturated brine solution. The organic layer is dried over magnesium sulfate and concentrated to 27.13 grams of a yellow oil that gradually solidified. The crude product is forward processed without purification in the next step.

Step 2 (2-(9H-fluoren-9-ylmethoxycarbonyloxy)acetic acid):

Tert-butyl 2-(9H-fluoren-9-ylmethoxycarbonyloxy)acetate (26.0 g, 73.40 mmol) from step 1 is dissolved in dichloromethane (200 mL). To the magnetically stirred solution is added trifluoroacetic acid (52 mL) followed by triisopropylsilane (13 mL). The resulting solution is stirred for 3 hours at ambient temperature. The solution is concentrated under reduced pressure to remove the dichloromethane and nearly all of the trifluoroacetic acid. The resulting viscous residue is treated gradually with 1000 mL of 5% aqueous sodium bicarbonate solution to prevent foaming and the aqueous solution is washed with 3×500 ml of methyl tert-butyl ether to remove residual triisopropylsilane. The aqueous solution is cooled to 0-5° C. and 300 ml of ethyl acetate are added. The biphasic mixture is acidified to ˜pH 2 with 40% aq. phosphoric acid, requiring about 75 ml of acid. After separating the layers, the organic layer is dried over magnesium sulfate and concentrated under reduced pressure to a viscous pale yellow oil. The oil is cooled to −20° C. in the freezer, which caused the material to completely solidify to a white solid. The solid is triturated with 75 ml of cold heptane and after sonication, a uniform white suspension formed. The solid is filtered off, washed with heptane, and dried in the vacuum oven overnight at 33° C. to afford 15.21 g (69.5% yield over two steps) of a white solid.

NMR (CDCl₃) confirmed that the desired product has been isolated.

### Example 73: Synthesis of Fmoc-Lys(Mtt)-Cys(Trt)-OH (Compound 60)

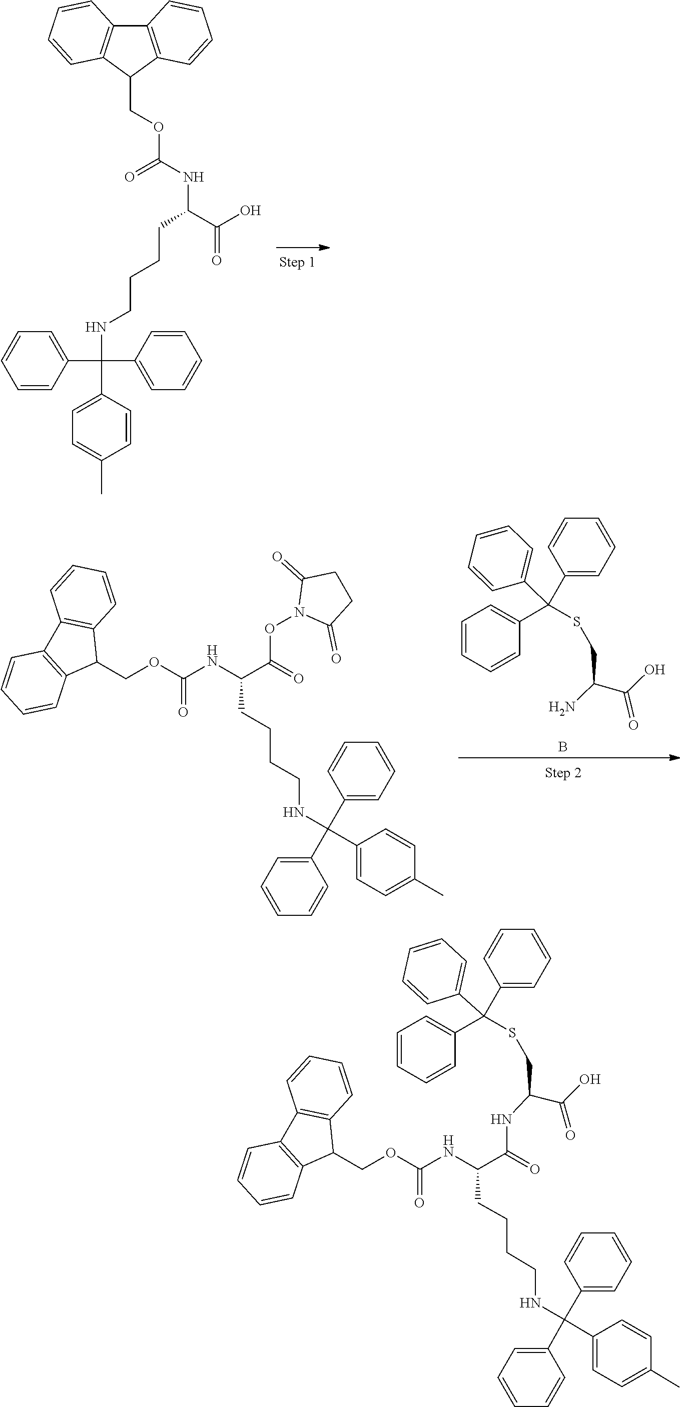

### Step 1

(2S)-6-[[diphenyl(p-tolyl)methyl]amino]-2-(9H-fluoren-9-ylmethoxycarbonylamino)hexanoic acid (A, 15 g, 24.01 mmol), N,N′-disuccinimidyl carbonate (7.45 g, 29.0 mmol, 99.6 mass %), 4-dimethyl aminopyridine [DMAP] (0.3 g, 2 mmol, 99 mass %) is weighed into 250 mL flask, equipped with a stir bar. Ethyl acetate (225 mL, 2000 mmol, 100 mass %) is then added and the solution mixed at room temperature (21-24° C.) until a solution is obtained. The reaction is stirred for 18 hours or until completion of reaction is confirmed by LCMS/NMR. The reaction mixture is transferred to a separatory funnel, washed with deionized water (60 mL×3) and the organic layer concentrated to dryness on the rotary evaporator to obtain crude compound of step 1.

### Step 2

To crude compound of step 1 (17.33 g, 24.01 mmol) in N,N-dimethylformamide (208 mL, 2690 mmol) is added N,N-diisopropylethylamine (5.03 mL, 28.8 mmol) and (2R)-2-amino-3-tritylsulfanyl-propanoic acid (9.6 g, 26 mmol). The reaction is stirred using magnetic stirring at room temperature (21-24° C.) for 18 hours or until completion of reaction is confirmed by LCMS/NMR. The reaction mixture is transferred to a separatory funnel, washed with 10% citric acid (120 mL×2) and extracted with dichloromethane (100 mL×5). The organic layer is washed with deionized water (100 mL×2) and the combined organic layers concentrated to dryness on the rotary evaporator at 48-50° C. to remove excess solvent. The crude Compound 60 is dissolved in Acetonitrile (30 mL) by sonication. The crude Compound 60 solution is added dropwise to cold acetonitrile: deionized water (3:2, 700 mL) while being stirred. The slurry is stirred at 0° C., overnight. The solid is filtered, washed with hexanes (70 mL) and dried in the vacuum oven at 40° C. to give the product Fmoc-Lys(Mtt)-Cys(Trt)-OH (Compound 60) (21.0 g, 76.8% yield corrected for potency by Q-NMR).

### Example 74: Synthesis of Fmoc-L-Lys(mtt)-L-Cys(trt)-L-Pro-glycolic acid-L-Val-OH (Compound 61)

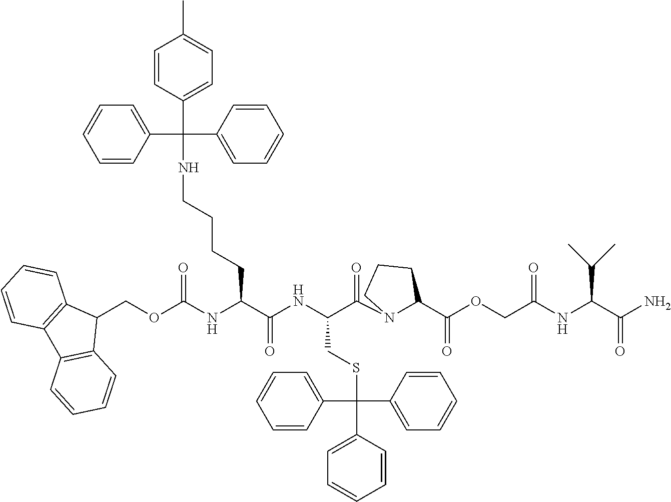

Prior to the coupling reaction, Fmoc-L-Pro-glycolic acid-L-Val-OH on resin from example 73 above (1.719 g, 1.00 mmol) is swelled with 3×15 ml of DMF for 20 minutes each, then deprotected with 4×15 ml of 20% piperidine/DMF (v/v) for 30 minutes each and washed with 5×15 ml of DMF for 1 minute each. A solution is made of (2R)-2-[[(2S)-6-[[diphenyl(p-tolyl)methyl]amino]-2-(9H-fluoren-9-ylmethoxycarbonylamino)hexanoyl]amino]-3-tritylsulfanyl-propanoic acid (2.28 g, 2.00 mmol, 85.24 mass %), hydroxy-3,4-dihydro-4-oxo-1,2,3-benzotriazine (HOOBt) (0.375 g, 2.30 mmol, 95 mass %), and N,N′-diisopropylcarbodiimide (0.41 ml, 2.60 mmol) in 10 ml of DMF. The corresponding solution is added to the reactor containing the resin. The reaction is mixed for 12 hours at ambient temperature and the liquid is drained. The resin is washed with 5×15 ml of DMF for 1 minute each and 5×15 ml of dichloromethane for 1 minute, then dried to constant weight to provide 1.973 g of the title compound on resin.

A 50 mg sample of the peptide is cleaved from the resin with 2.0 mL of a solution consisting of 92.5% TFA, 2.5% triisopropylsilane, 2.5% water, and 2.5% dithiothreitol (v/v/v/w). The mixture is agitated on a rotary mixer for 1.5 hours, diluted with 16 ml of 80:20 DMSO/acetonitrile (v/v), and filtered to remove the resin. The filtrate is analyzed by LC/MS and shown to contain 81.93 area % desired pentapeptide along with 2.91 area % of des-Proline and 3.83% of des-Valine.

### Example 75: Synthesis of Fmoc-Gly-Pro-Ser(tBu)-Ser(tBu)-Gly-Ala-Pro-Pro-Pro-Ser(tBu)-OH (SEQ ID NO:55)

The title compound is prepared using standard solid phase synthesis conditions (Fmoc-protected amino acids/ethyl cyanoglyoxylate-2-oxime (Oxyma)/N,N′-diisopropylcarbodiimide (DIC) as described below.

### Solvent and Reagents Preparations:

Twenty L of DMF are charged to the solvent reservoir. Five L of 20% Piperidine/DMF (v/v) solution are charged to the deprotection reservoir. 600 mL of 0.660 M DIC solution is prepared using N,N′-diisopropylcarbodiimide (49.98 g, 396.0 mmol) and DMF and charged to the DIC/solvent reservoir. 500 ml of 0.750 M Oxyma solution is prepared using ethyl cyanoglyoxylate-2-oxime (53.29 g, 371.2 mmol) and DMF and is charged to the Oxyma/solvent reservoir. Sieber resin (0.71 mmol/g, 14.09 g, 10.00 mmol) is charged to the reactor. Prior to beginning the synthetic steps shown below, the resin is swelled with 3×180 ml of DMF for 20 minutes each and the Fmoc group is removed with 3×180 ml of 20% piperidine/DMF (v/v) for 30 minutes each.

### Amino Acid Solution Preparations:

One hundred mL of 0.375 M FmocNH-L-Ala-OH solution is prepared from (2S)-2-(9H-fluoren-9-ylmethoxycarbonylamino)propanoic acid (11.68 g, 37.52 mmol) and DMF and charged to the appropriate amino acid bottle. 200 mL of 0.375 M FmocNH-Gly-OH solution is prepared from 2-(9H-fluoren-9-ylmethoxycarbonylamino)acetic acid (22.30 g, 75.01 mmol) and DMF and charged to the appropriate amino acid bottle. 360 mL of 0.375 M FmocNH-L-Pro-OH solution is prepared from (2S)-1-(9H-fluoren-9-ylmethoxycarbonyl)pyrrolidine-2-carboxylic acid (45.54 g, 135.0 mmol) and DMF and charged to the appropriate amino acid bottle. 280 mL of 0.375 M FmocNH-L-Ser(tBu)-OH solution is prepared from (2S)-3-tert-butoxy-2-(9H-fluoren-9-ylmethoxycarbonylamino)propanoic acid (40.25 g, 105.0 mmol) and DMF and charged to the appropriate amino acid bottle.

### Coupling Conditions:

Pro: 0.18 M, 3.0 equiv amino acid, 3.0 equiv Oxyma/3.3 equiv DIC, 30 minute pre-activation of activated ester solution, 6 hour coupling time at ambient temperature, 4×30 minute deprotection with 20% piperidine/DMF (v/v), 5×2 minute DMF washes post deprotection and post-coupling.

Ala (after Pro), Gly (after Pro): 0.18 M, 3.0 equiv amino acid, 3.0 equiv Oxyma/3.3 equiv DIC, 30 minute pre-activation of activated ester solution, 4 hour coupling time at ambient temperature, 4×30 minute deprotection with 20% piperidine/DMF (v/v), 5×2 minute DMF washes post deprotection and post-coupling.

All other couplings: 0.18 M, 3.0 equiv amino acid, 3.0 equiv Oxyma/3.3 equiv DIC, 30 minute pre-activation of activated ester solution, 4 hour coupling time at ambient temperature, 3×30 minute deprotection with 20% piperidine/DMF (v/v), 5×2 minute DMF washes post deprotection and post-coupling.

At the end of the synthesis, the resin is washed with 5×180 ml of DMF for 2 minutes each, followed by 5×180 ml of MTBE for 2 minutes each. The resin is removed from the reactor, transferred to a tared crystallization dish, and dried in vacuo at 40° C. to constant weight to provide 25.15 g of the title compound on resin. Based upon the mass of the resin starting material, the yield of peptide is 11.07 g (89%).

The Fmoc group is removed from a 251 mg sample of the peptide on resin by swelling the resin with 3×6 ml of DMF for 10 minutes, treating with 3×6 ml of 20% piperidine/DMF (v/v) for 30 minutes each, washing with 5×6 ml of DMF for 1 minute each, washing with 5×6 ml of dichloromethane for 1 minute each and drying to constant weight.

The deprotected product sample is cleaved from the resin by mixing on a rotary mixer in a 20 ml scintillation vial for 2 hours with 5 mL of TFA/TIS/H2O/DTT ([0.925 v:0.025 v:0.025 v]:0.025 w) solution. The resin is filtered and the resin wet cake is washed with 2 mL of neat TFA.

The resulting crude peptide is precipitated with 35 mL of cold MTBE, centrifuged, washed with 2×35 ml of MTBE, and dried in vacuo overnight at 33° C. to give 105.1 mg (94.9%) of the fully deprotected peptide. Analysis by UPLC showed 98.62 area % purity with no related substances over 0.30 area %. The loading of the peptide on resin is measured at 0.37 mmol/g vs theoretical loading of 0.37 mmol/g.

### Example 75: Synthesis of H-Cys-Gln-Aib-Phe-Ile-Glu-Tyr-Leu-Leu-Glu-Gly-Gly-Pro-Ser-Ser-Gly-Ala-Pro-Pro-Pro-Ser-NH₂ SEQ ID NO:52 (Compound 62)

The title compound is prepared using standard solid phase synthesis conditions (Fmoc-protected amino acids/ethyl cyanoglyoxylate-2-oxime (Oxyma)/N,N′-diisopropylcarbodiimide (DIC) as described below.

### Solvent and Reagents Preparations:

Forty L of DMF are charged to the solvent reservoir. 4 L of 20% Piperidine/DMF (v/v) solution are charged to the deprotection reservoir. 600 mL of 0.660 M DIC solution is prepared using N,N′-diisopropylcarbodiimide (49.98 g, 396.0 mmol) and DMF and charged to the DIC/solvent reservoir. 500 ml of 0.750 M Oxyma solution is prepared using ethyl cyanoglyoxylate-2-oxime (53.29 g, 371.2 mmol) and DMF and charged to the Oxyma/solvent reservoir. 9H-fluoren-9-ylmethyl N-[2-[(2S)-2-[[(1S)-2-[[(1S)-2-[[2-[[(1S)-2-[(2S)-2-[(2S)-2-[(2S)-2-[[(1S)-2-amino-1-(tert-butoxymethyl)-2-oxo-ethyl]carbamoyl]pyrrolidine-1-carbonyl]pyrrolidine-1-carbonyl]pyrrolidin-1-yl]-1-methyl-2-oxo-ethyl]amino]-2-oxo-ethyl]amino]-1-(tert-butoxymethyl)-2-oxo-ethyl]amino]-1-(tert-butoxymethyl)-2-oxo-ethyl]carbamoyl]pyrrolidin-1-yl]-2-oxo-ethyl]carbamate on Sieber resin (0.41 mmol/g, 1.22 g, 0.500 mmol) is charged to each of eleven reactors (total of 5.5 mmol of peptide on resin). Prior to beginning the synthetic steps shown below, the resin in each reactor is swelled with 3×10 ml of DMF for 20 minutes each then the Fmoc group is removed with 3×10 ml of 20% piperidine/DMF (v/v) for 30 minutes each and the resin is washed with 5×10 ml of DMF for 1 minute each.

### Amino Acid Solution Preparations:

1. 57 mL of 0.375 M FmocNH-L-Ala-OH solution is prepared from (2S)-2-(9H-fluoren-9-ylmethoxycarbonylamino)propanoic acid (6.66 g, 21.38 mmol) and DMF and charged to the appropriate amino acid bottle.

2. 103 mL of 0.375 M FmocNH-L-Glu(tBu)-OH solution is prepared from (2S)-5-tert-butoxy-2-(9H-fluoren-9-ylmethoxycarbonylamino)-5-oxo-pentanoic acid (16.44 g, 38.63 mmol) and DMF and charged to the appropriate amino acid bottle.

3. 61 mL of 0.375 M FmocNH-L-Phe-OH solution is prepared from (2S)-2-(9H-fluoren-9-ylmethoxycarbonylamino)-3-phenyl-propanoic acid (8.83 g, 22.79 mmol) and DMF and charged to the appropriate amino acid bottle.

4. 57 mL of 0.375 M FmocNH-Gly-OH solution is prepared from 2-(9H-fluoren-9-ylmethoxycarbonylamino)acetic acid (6.36 g, 21.38 mmol) and DMF and charged to the appropriate amino acid bottle.

5. 57 mL of 0.375 M FmocNH-L-Ile-OH solution is prepared from (2S,3S)-2-(9H-fluoren-9-ylmethoxycarbonylamino)-3-methyl-pentanoic acid (7.55 g, 21.38 mmol) and DMF and charged to the appropriate amino acid bottle.

6. 103 mL of 0.375 M FmocNH-L-Leu-OH solution is prepared from (2S)-2-(9H-fluoren-9-ylmethoxycarbonylamino)-4-methyl-pentanoic acid (13.65 g, 38.62 mmol) and DMF and charged to the appropriate amino acid bottle.

7. 82 mL of 0.375 M FmocNH-L-Gln(trt)-OH solution is prepared from (2S)-5-(tert-butylamino)-2-(9H-fluoren-9-ylmethoxycarbonylamino)-5-oxo-pentanoic acid (13.05 g, 21.38 mmol) and DMF and charged to the appropriate amino acid bottle.

8. 57 mL of 0.375 M FmocNH-L-Tyr(tBu)-OH solution is prepared from (2S)-3-(4-tert-butoxyphenyl)-2-(9H-fluoren-9-ylmethoxycarbonylamino)propanoic acid (9.82 g, 21.38 mmol) and DMF and charged to the appropriate amino acid bottle.

9. 57 mL of 0.375 M FmocNH-Aib-OH solution is prepared from 2-(9H-fluoren-9-ylmethoxycarbonylamino)-2-methyl-propanoic acid (6.96 g, 21.38 mmol) and DMF and charged to the appropriate amino acid bottle.

10. 44 mL of 0.375 M FmocNH-L-Cys(trt)-OH solution is prepared from (2R)-2-(9H-fluoren-9-ylmethoxycarbonylamino)-3-tritylsulfanyl-propanoic acid (9.66 g, 16.50 mmol) and DMF and charged to the appropriate amino acid bottle.

### Coupling Conditions:

Gln: 0.18 M, 3.0 equiv amino acid, 3.0 equiv Oxyma/3.3 equiv DIC, 30 minute pre-activation of activated ester solution, 18 hour coupling time at ambient temperature, 4×30 minute deprotection with 20% piperidine/DMF (v/v), 5×1 minute DMF washes post deprotection and post-coupling.

Aib, Ala, Leu-9: 0.18 M, 3.0 equiv amino acid, 3.0 equiv Oxyma/3.3 equiv DIC, 30 minute pre-activation of activated ester solution, 12 hour coupling time at ambient temperature, 4×30 minute deprotection with 20% piperidine/DMF (v/v), 5×1 minute DMF washes post deprotection and post-coupling.

Phe, Ile: 0.18 M, 3.0 equiv amino acid, 3.0 equiv Oxyma/3.3 equiv DIC, 30 minute pre-activation of activated ester solution, 8 hour coupling time at ambient temperature, 4×30 minute deprotection with 20% piperidine/DMF (v/v), 5×1 minute DMF washes post deprotection and post-coupling.

Cys: 0.18 M, 3.0 equiv amino acid, 3.0 equiv Oxyma/3.3 equiv DIC, no pre-activation of activated ester solution, 8 hour coupling time at ambient temperature, 4×30 minute deprotection with 20% piperidine/DMF (v/v), 5×1 minute DMF washes post deprotection and post-coupling.

All other couplings: 0.18 M, 3.0 equiv amino acid, 3.0 equiv Oxyma/3.3 equiv DIC, 30 minute pre-activation of activated ester solution, 4 hour coupling time at ambient temperature, 3×30 minute deprotection with 20% piperidine/DMF (v/v), 5×1 minute DMF washes post deprotection and post-coupling.

At the end of the synthesis, each resin is washed with 5×10 ml of DMF for 1 minute each, followed by 5×1 ml of dichloromethane for 1 minute each. The resins are dried to constant weight and combined to provide 24.394 g of the title compound on resin. The Fmoc group is removed from a 91.8 mg sample of the peptide on resin by swelling the resin with 3×4 ml of DMF for 15 minutes each, treating with 3×4 ml of 20% piperidine/DMF (v/v) for 30 minutes each, washing with 5×4 ml of DMF for 1 minute each, washing with 5×4 ml of dichloromethane for 1 minute each and drying to constant weight. The deprotected product sample is cleaved from the resin by mixing on a rotary mixer in a 20 ml scintillation vial for 2 hours with 5 mL of TFA/TIS/H₂O/DTT ([0.925 v:0.025 v:0.025 v]:0.025 w) solution. The resin is filtered and the resin wet cake is washed with 2 mL of neat TFA. The resulting crude peptide is precipitated with 35 mL of cold MTBE, centrifuged, washed with 2×35 ml of MTBE, and dried in vacuo overnight at 33° C. to give 48.2 mg of the fully deprotected peptide. Analysis by UPLC showed 88.02 area % purity with no related substances over 1.0 area %. The remainder of the peptide is cleaved from the resin by mechanically stirring with 200 ml of a solution made up of 185 mL of trifluoroacetic acid, 5.0 mL of triisopropylsilane, 5.0 mL of water, 5.0 g of dithiothreitol in a 3-necked round bottomed flask for 2 hours at ambient temperature. The resin is removed by filtration on a fritted funnel and washed with 80 ml of TFA to give a total solution volume of approximately 280 mL. The peptide is precipitated by addition to 1400 ml of cold MTBE. After aging at −20° C. for 1 hour, the slurry is divided into six bottles and centrifuged. The resulting solids after centrifugation are combined into two bottles and each solid is washed twice with 250 ml of room temperature MTBE. The resulting white solid is dried overnight in the vacuum oven at 33° C. to give 20.07 g of crude peptide.

### Example 76: Synthesis of Boc-Tyr(tBu)-Aib-Gln(trt)-Glu(tBu)-Thr(tBu)-Phe-Thr(tBu)-Ser(tBu)-Asp(tBu)-Tyr(tBu)-Ser(tBu)-Ile-αMeLeu-Leu-Asp(tBu)-Lys(mtt)-Cys(trt)-Pro-glycolic acid-Val-NH₂ (SEQ ID NO:56; Compound 63)

The title compound is prepared using standard solid phase synthesis conditions (Fmoc-protected amino acids/ethyl cyanoglyoxylate-2-oxime (Oxyma)/N,N′-diisopropylcarbodiimide (DIC).

### Solvent and Reagents Preparations:

Forty L of DMF are charged to the solvent reservoir. 4 L of 20% Piperidine/DMF (v/v) solution are charged to the deprotection reservoir. 600 mL of 0.660 M DIC solution is prepared using N,N′-diisopropylcarbodiimide (49.98 g, 396.0 mmol) and DMF and charged to the DIC/solvent reservoir. 500 ml of 0.750 M Oxyma solution is prepared using ethyl cyanoglyoxylate-2-oxime (53.29 g, 371.2 mmol) and DMF and charged to the Oxyma/solvent reservoir. [2-[[(1S)-1-carbamoyl-2-methyl-propyl]amino]-2-oxo-ethyl] (2S)-1-[(2R)-2-[[(2S)-6-[[diphenyl(p-tolyl)methyl]amino]-2-(9H-fluoren-9-ylmethoxycarbonylamino)hexanoyl]amino]-3-trityl sulfanyl-propanoyl]pyrrolidine-2-carboxylate on Rink Amide AM, Rink Amide MBHA or Sieber resin (0.500 mmol) is charged to each of eight reactors (total of 4.0 mmol of peptide on resin).

Prior to beginning the synthetic steps shown below, the resin in each reactor is swelled with 3×10 ml of DMF for 20 minutes each then the Fmoc group is removed with 4×10 ml of 20% piperidine/DMF (v/v) for 30 minutes each and the resin was washed with 5×10 ml of DMF for 1 minute each.

### Amino Acid Solution Preparations:

1. 57 mL of 0.375 M FmocNH-Aib-OH solution is prepared from 2-(9H-fluoren-9-ylmethoxycarbonylamino)-2-methyl-propanoic acid (6.96 g, 21.37 mmol) and DMF, then charged to the appropriate amino acid bottle.

2. 103 mL of 0.375 M FmocNH-L-Asp(tBu)-OH solution is prepared from (2S)-4-tert-butoxy-2-(9H-fluoren-9-ylmethoxycarbonylamino)-4-oxo-butanoic acid (15.87 g, 38.63 mmol) and DMF, then charged to the appropriate amino acid bottle.

3. 57 mL of 0.375 M FmocNH-L-Phe-OH solution is prepared from (2S)-2-(9H-fluoren-9-ylmethoxycarbonylamino)-3-phenyl-propanoic acid (8.28 g, 21.38 mmol) and DMF, then charged to the appropriate amino acid bottle.

4. 57 mL of 0.375 M FmocNH-Gly-OH solution is prepared from 2-(9H-fluoren-9-ylmethoxycarbonylamino)acetic acid (6.36 g, 21.38 mmol) and DMF, then charged to the appropriate amino acid bottle.

5. 57 mL of 0.375 M FmocNH-L-Ile-OH solution is prepared from (2S,3S)-2-(9H-fluoren-9-ylmethoxycarbonylamino)-3-methyl-pentanoic acid (7.55 g, 21.38 mmol) and DMF, then charged to the appropriate amino acid bottle.

6. 57 mL of 0.375 M FmocNH-L-Lys(boc)-OH solution is prepared from (2S)-6-(tert-butoxycarbonylamino)-2-(9H-fluoren-9-ylmethoxycarbonylamino)hexanoic acid (10.02 g, 21.38 mmol) and DMF, then charged to the appropriate amino acid bottle.

7. 57 mL of 0.375 M FmocNH-L-Leu-OH solution is prepared from (2S)-2-(9H-fluoren-9-ylmethoxycarbonylamino)-4-methyl-pentanoic acid (7.55 g, 21.38 mmol) and DMF, then charged to the appropriate amino acid bottle.

8. 57 mL of 0.375 M FmocNH-L-Gln(trt)-OH solution is prepared from (2S)-5-(tert-butylamino)-2-(9H-fluoren-9-ylmethoxycarbonylamino)-5-oxo-pentanoic acid (13.05 g, 21.38 mmol) and DMF, then charged to the appropriate amino acid bottle.

9. 103 mL of 0.375 M FmocNH-L-Ser(tBu)-OH solution is prepared from (2S)-3-tert-butoxy-2-(9H-fluoren-9-ylmethoxycarbonylamino)propanoic acid (14.81 g, 38.63 mmol) and DMF, then charged to the appropriate amino acid bottle.

10. 103 mL of 0.375 M FmocNH-L-Thr(tBu)-OH solution is prepared from (2S,3R)-3-tert-butoxy-2-(9H-fluoren-9-ylmethoxycarbonylamino)butanoic acid (15.35 g, 38.63 mmol) and DMF, then charged to the appropriate amino acid bottle.

11. 57 mL of 0.375 M FmocNH-L-Tyr(tBu)-OH solution is prepared from (2S)-3-(4-tert-butoxyphenyl)-2-(9H-fluoren-9-ylmethoxycarbonylamino)propanoic acid (9.82 g, 21.38 mmol) and DMF, then charged to the appropriate amino acid bottle.

12. 57 mL of 0.375 M BocNH-L-Tyr(tBu)-OH solution is prepared from (2S)-2-(tert-butoxycarbonylamino)-3-(4-tert-butoxyphenyl)propanoic acid (7.21 g, 21.38 mmol) and DMF, then charged to the appropriate amino acid bottle.

13. 57 mL of 0.375 M FmocNH-L-αMeLeu-OH solution is prepared from (2R)-2-(9H-fluoren-9-ylmethoxycarbonylamino)-2,4-dimethyl-pentanoic acid (7.85 g, 21.37 mmol) and DMF, then charged to the appropriate amino acid bottle.

### Coupling Conditions:

Boc-Tyr, Ile: 0.18 M, 3.0 equiv amino acid, 3.0 equiv Oxyma/3.3 equiv DIC, 30 minute pre-activation of activated ester solution, 18 hour coupling time at ambient temperature, 4×30 minute deprotection with 20% piperidine/DMF (v/v), 5×1 minute DMF washes post deprotection and post-coupling.

Aib, Gln, αMeLeu: 0.18 M, 3.0 equiv amino acid, 3.0 equiv Oxyma/3.3 equiv DIC, 30 minute pre-activation of activated ester solution, 12 hour coupling time at ambient temperature, 4×30 minute deprotection with 20% piperidine/DMF (v/v), 5×1 minute DMF washes post deprotection and post-coupling.

All other couplings: 0.18 M, 3.0 equiv amino acid, 3.0 equiv Oxyma/3.3 equiv DIC, 30 minute pre-activation of activated ester solution, 4 hour coupling time at ambient temperature, 3×30 minute deprotection with 20% piperidine/DMF (v/v), 5×1 minute DMF washes post deprotection and post-coupling. At the end of the synthesis, each resin is washed with 5×10 ml of DMF for 1 minute each, followed by 5×1 ml of dichloromethane for 1 minute each. The resins are dried to constant weight and forward processed to the next step. A sample from one of the reactors (˜80 mg) is cleaved from the resin by mixing on a rotary mixer in a 20 ml scintillation vial for 2 hours with 5 mL of TFA/TIS/H2O/DTT ([0.925 v:0.025 v:0.025 v]:0.025 w) solution. The resin is filtered and the resin wet cake is washed with 2 mL of neat TFA. The resulting crude peptide is precipitated with 35 mL of cold MTBE, centrifuged, washed with 2×35 ml of MTBE, and dried in vacuo overnight at 33° C. to give a sample of the fully deprotected peptide. Analysis by UPLC showed 59.8 area % purity.

### Example 77: Synthesis of Tyr-Aib-Gln-Glu-Thr-Phe-Thr-Ser-Asp-Tyr-Ser-Ile-αMeLeu-Leu-Asp-Lys(AEEA-AEEA-γGlu-C₂₀-OH)-Cys-Pro-glycolic acid-Val-NH₂ (SEQ ID NO:57; Compound 64)

### Step 1 (Deprotection of mtt Protecting Group):

[2-[[(1S)-1-carbamoyl-2-methyl-propyl]amino]-2-oxo-ethyl] (2S)-1-[(2R)-2-[[(2S)-2-[[(2S)-2-[[(2S)-4-tert-butoxy-2-[[(2S)-2-[[(2S)-2-[[(2S,3 S)-2-[[(2S)-3-tert-butoxy-2-[[(2S)-2-[[(2S)-4-tert-butoxy-2-[[(2S)-3-tert-butoxy-2-[[(2S,3R)-3-tert-butoxy-2-[[(2S)-2-[[(2S,3R)-3-tert-butoxy-2-[[2-[[(2S)-2-[[2-[[(2S)-2-(tert-butoxycarbonylamino)-3-(4-tert-butoxyphenyl)propanoyl]amino]-2-methyl-propanoyl]amino]-5-oxo-5-(tritylamino)pentanoyl]amino]acetyl]amino]butanoyl]amino]-3-phenyl-propanoyl]amino]butanoyl]amino]propanoyl]amino]-4-oxo-butanoyl]amino]-3-(4-tert-butoxyphenyl)propanoyl]amino]propanoyl]amino]-3-methyl-pentanoyl]amino]-2,4-dimethyl-pentanoyl]amino]-4-methyl-pentanoyl]amino]-4-oxo-butanoyl]amino]-6-(tert-butoxycarbonylamino)hexanoyl]amino]-6-[[diphenyl(p-tolyl)methyl]amino]hexanoyl]amino]-3-tritylsulfanyl-propanoyl]pyrrolidine-2-carboxylate on Rink Amide AM, Rink Amide MBHA or Sieber resin (0.500 mmol) is charged to each of eight different reactors. Each resin is swelled with 3×10 ml of DCM for 15 min each and then treated with 1,1,1,3,3,3-hexafluoro-2-propanol, 30% in dichloromethane (v/v) (10 mL, 94.98 mmol) and mixed for one hour. The liquid is drained and the resin is again treated with 1,1,1,3,3,3-hexafluoro-2-propanol, 30% in dichloromethane (v/v) (10 mL, 94.98 mmol) and mixed for one hour. The liquid is again drained and the resin is washed with 5×10 ml of dichloromethane for 1 minute each, then 5×10 ml of DMF for 1 minute each and forward processed to the coupling reaction.

### Step 2 (Coupling of Sidechain):

2-[2-[2-[[(4S)-5-tert-butoxy-4-[(20-tert-butoxy-20-oxo-icosanoyl)amino]-5-oxo-pentanoyl]amino]ethoxy]ethoxy]acetic acid (6.41 g, 8.00 mmol, 91 mass %) and benzotriazol-1-yl-oxytripyrrolidinophosphonium hexafluorophosphate (PyBOP) (4.16 g, 8.00 mmol) are dissolved in 72 mL of DMF. N,N-diisopropylethylamine (1.40 mL, 8.00 mmol) is added and the resulting solution is shaken for 1 minute, then one-eighth of the solution is added each resin in the reaction vessels and mixed for 16 hours. The liquid is drained and the resin is washed with 5×15 ml of DMF for 1 minute each, 5×15 ml of dichloromethane for 1 minute each, and then dried to constant weight to afford 15.66 g of the peptide on resin.

Step 3 (Cleavage and Global Deprotection of Peptide from Resin):

The peptide is cleaved from the resin by mechanically stirring with 160 ml of a solution made up of 148 mL of trifluoroacetic acid, 4.0 mL of triisopropylsilane, 4.0 mL of water, and 4.0 g of dithiothreitol in a 3-necked round bottomed flask for 2 hours at ambient temperature. The resin is removed by filtration on a fritted funnel and washed with 64 ml of TFA to give a total solution volume of approximately 224 mL. The peptide is precipitated by addition to 1120 ml of cold MTBE. After aging at −20° C. for 1 hour, the slurry is divided into four bottles and centrifuged. The resulting solids after centrifugation are combined into two bottles and each solid is washed twice with 250 ml of room temperature MTBE. The resulting solid is dried overnight in the vacuum oven at 33° C. to give 7.817 g of crude title compound.

### Example 78: Purification of SEQ ID NO:6

The crude product (76.23 g) is dissolved in 3.05 L of 25% ACN/Water mixture (25 g/L crude concentration) in a 5 L reactor and stirred for 30 min. Remediate depsipeptide isomers by conversion using 28% ammonium hydroxide adjusting pH=9.0 and stir for 60 min, followed by a subsequent adjustment back to the acidic side (pH=2 using TFA). The final ACN content needs to be 30% to ensure solubility after the pH adjustment. Filter the crude prior to the first chromatography step.

First Chromatography Step: Column: DAC200, 200 mm×250 mm, stationary phase (YMC Triart C18, 10 um, 12 nm); Mobile phase A: 0.1% TFA in H₂O; Mobile phase B: 100% ACN; detection at 230 nm; Injection volume: 3.5 L (by injection pump with the flow of 300 ml/min).

Gradient:

| Time/min | Flow/(ml/min) | % A | % B |
|---|---|---|---|
| 0 | 1000 | 70 | 30 |
| 1 | 1000 | 70 | 30 |
| 52.5 | 1000 | 45 | 55 |

Collect fractions with desired product.

Second Chromatography Step: dilute with equal volume of H₂O, and adjust pH to 6.5 using diluted ammonia. Column: DAC200, 200 mm×250 mm, stationary phase (YMC Triart C18, 10 um, 12 nm). Mobile phase A: 10 mM NH₄HCO₃ in H₂O; Mobile phase B: 100% ACN; detection at 230 nm; Injection volume: 7.0 L (by injection pump with the flow of 300 ml/min).

Gradient:

| Time/min | Flow/(ml/min) | % A | % B |
|---|---|---|---|
| 0 | 1000 | 70 | 30 |
| 1 | 1000 | 70 | 30 |
| 52.5 | 1000 | 45 | 55 |

Collect fractions with desired product.

Sodium Salt conversion step: Charge 1.76 g (44.0 mmol) NaOH dissolved in 200 ml H₂O dropwise into the 7.2 L of the separated fraction and lyophilize. Obtain 38.02 g purified product (purity:98.0%).

### Example 79: Purification of SEQ ID NO:29

The crude product is purified using a 20 cm column (4.8 kg Daiso C18-ODS-RPS, 10μ, 120 Å) and Mobile phase A: 0.1% TFA in H₂O; Mobile phase B: 100% ACN; detection at 230 nm. The first purification step:

Gradient:

| Time/min | Flow/(ml/min) | % A | % B |
|---|---|---|---|
| 0 | 600 | 80 | 20 |
| 3 | 600 | 70 | 30 |
| 75 | 600 | 45 | 55 |

Collect fractions with product of about 88% or greater purity, dilute 1:1 with H₂O and adjust pH to 6.5 with 50% NH₄OH.

Second chromatography step: use column in first step. Mobile phase A: 10 mM NH₄HCO₃ in H₂O; Mobile phase B: 100% ACN; detection at 230 nm.

Gradient:

| Time/min | Flow/(ml/min) | % A | % B |
|---|---|---|---|
| 0 | 600 | 80 | 20 |
| 45 | 600 | 50 | 50 |

Collect fractions with purity of about 97% or greater purity.

Convert to the sodium salt by adding 3 eq of NaOH followed by lyophilization.

### SEQUENCES

The following amino acid sequences are referred to in the disclosure and are provided below for reference.

human GIP
SEQ ID NO: 1
YAEGTFISDYSIAMDKIHQQDFVNWLLAQKGKKNDWKHNITQ
human GLP-1 (7-36) amide
SEQ ID NO: 2
HAEGTFTSDVSSYLEGQAAKEFIAWLVKGR-NH₂
human GCG
SEQ ID NO: 3
HSQGTFTSDYSKYLDSRRAQDFVQWLMNT

SEQ ID NO:4—Incretin analog
YX₂QGTFTSDYSIX₁₃LDKX₁₇AX₁₉X₂₀AFIEYLLX₂₈X₂₉GPSSX₃₄APPPS, where X₂ is Aib, X₁₃ is L or αMeL, X₁₇ is any amino acid with a functional group available for conjugation, and the functional group is conjugated to a C₁₆-C₂₂ fatty acid, X₁₉ is Q or A, X₂₀ is Aib, αMeK, Q or H, X₂₈ is E or A, X₂₉ is G or Aib, X₃₄ is G or Aib, and the C-terminal amino acid is optionally amidated.

Incretin analog
SEQ ID NO: 5
Y(Aib)QGTFTSDYSI(αMeL)LDKKAQ(Aib)AFIEYLLEGGPSSGAPP
PS
Incretin analog
SEQ ID NO: 6
Y(Aib)QGTFTSDYSI(αMeL)LDKK((2-[2-(2-amino-ethoxy)-
ethoxy]-acetyl)-(γGlu)-CO-(CH₂)₁₈-CO₂H)AQ(Aib)AFIE
YLLEGGPSSGAPPPS-NH₂

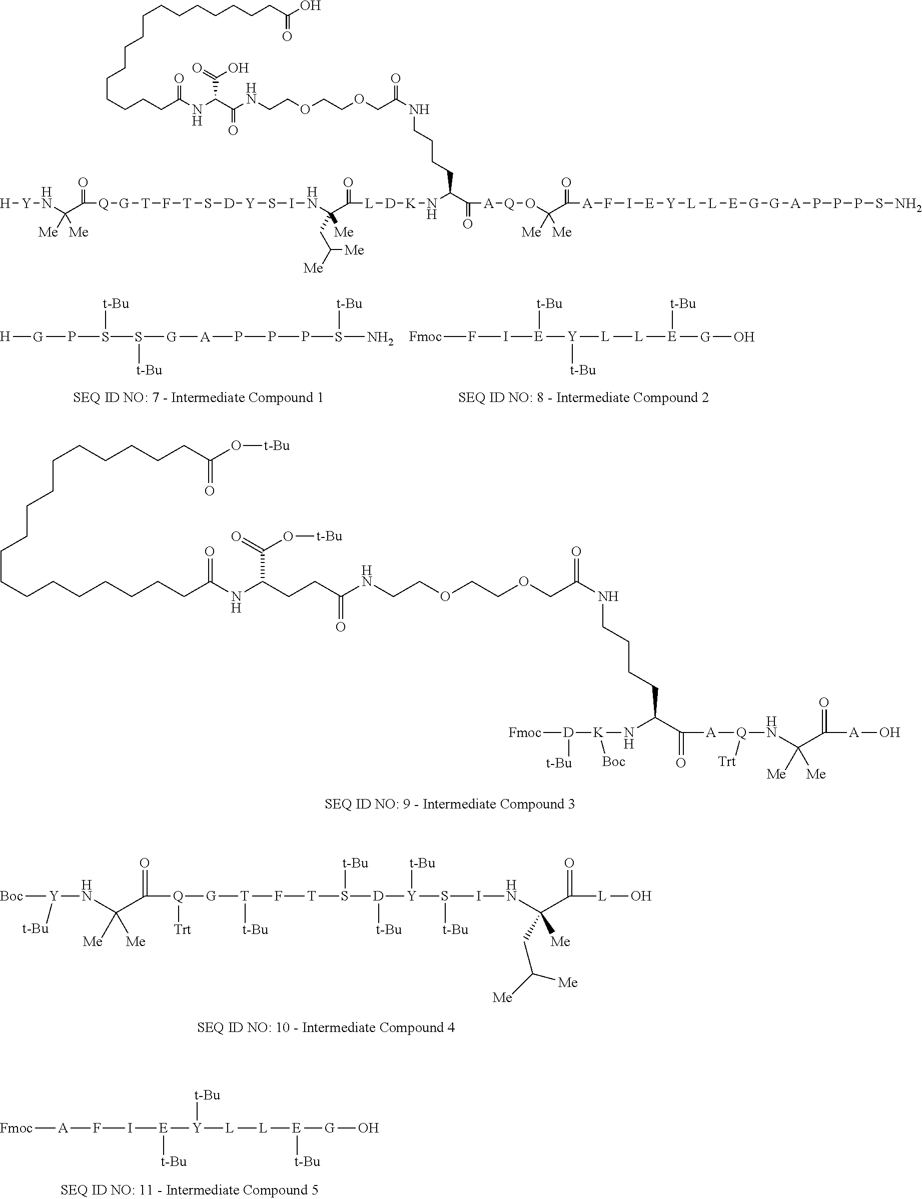

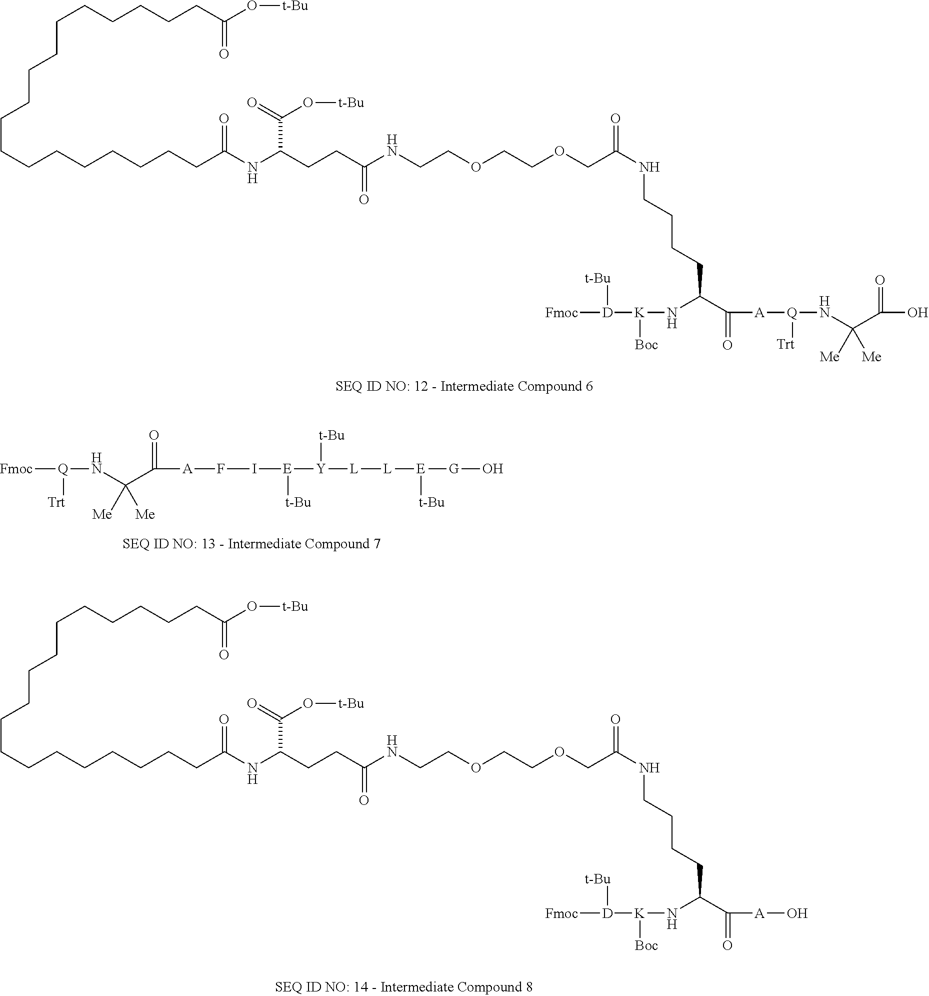

where R can be 2,2,2-trifluoroethyl.

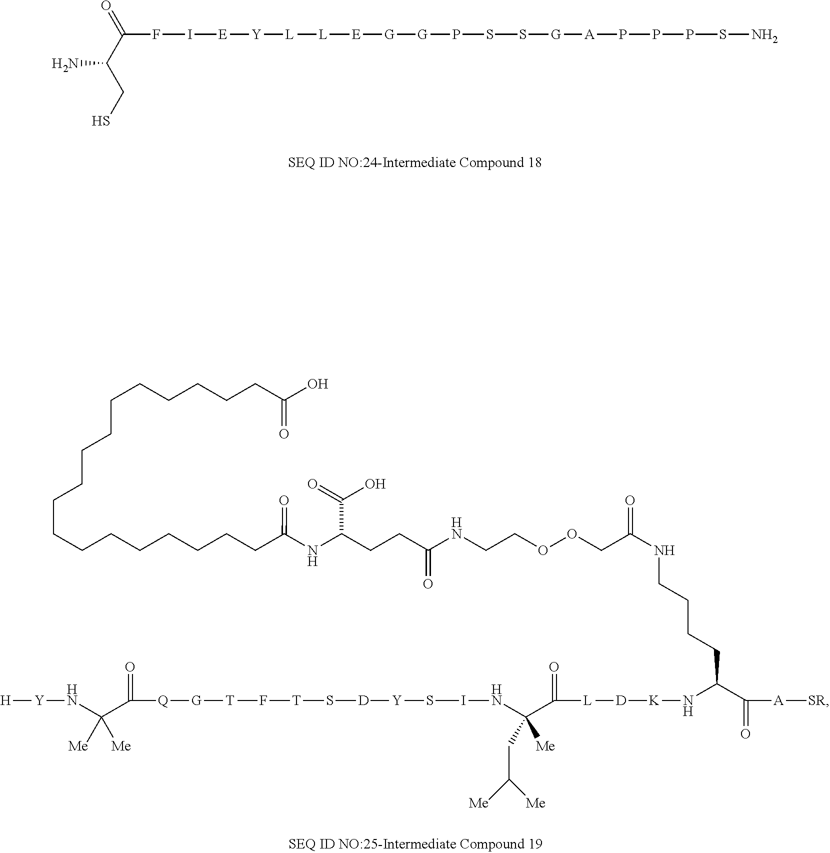

where R may be 2,2,2-trifluoroethyl.

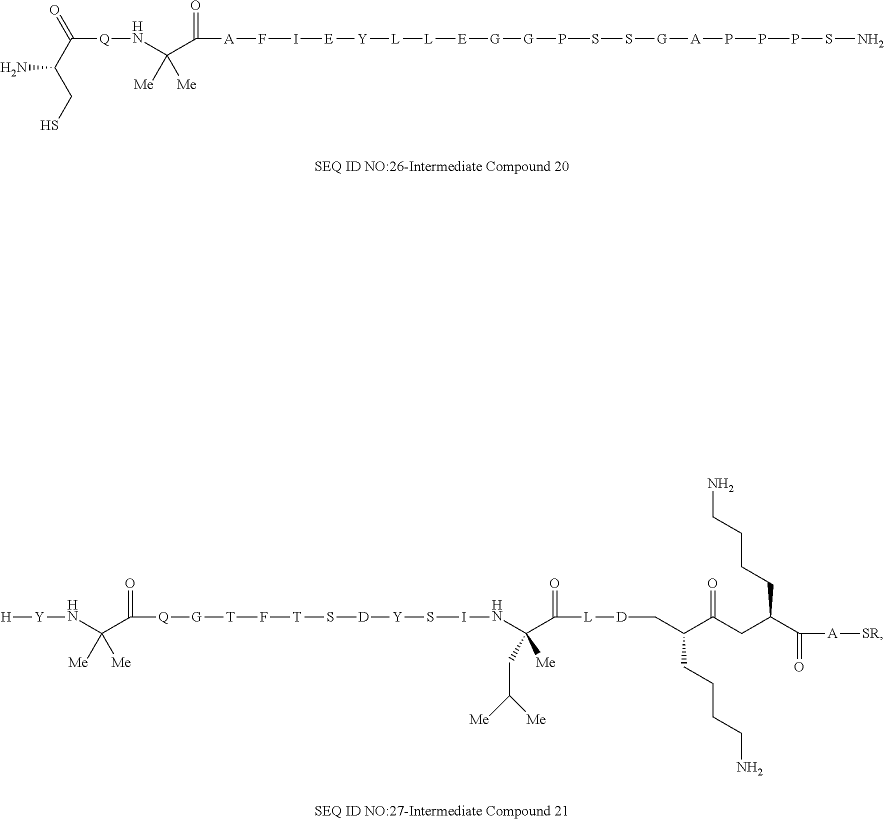

where R may be 2,2,2-trifluoroethyl.

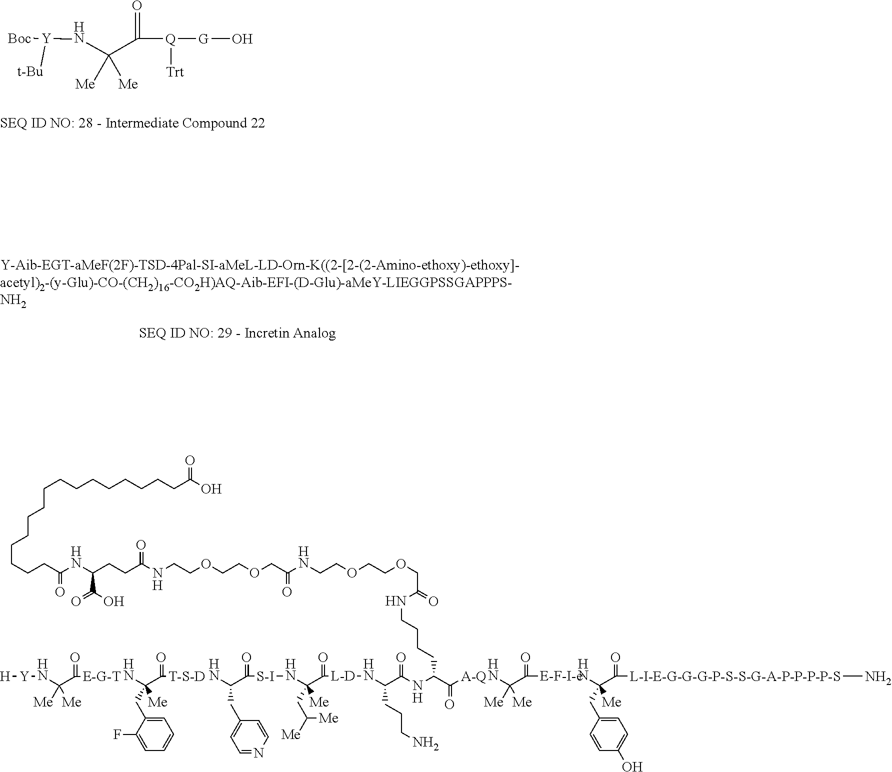

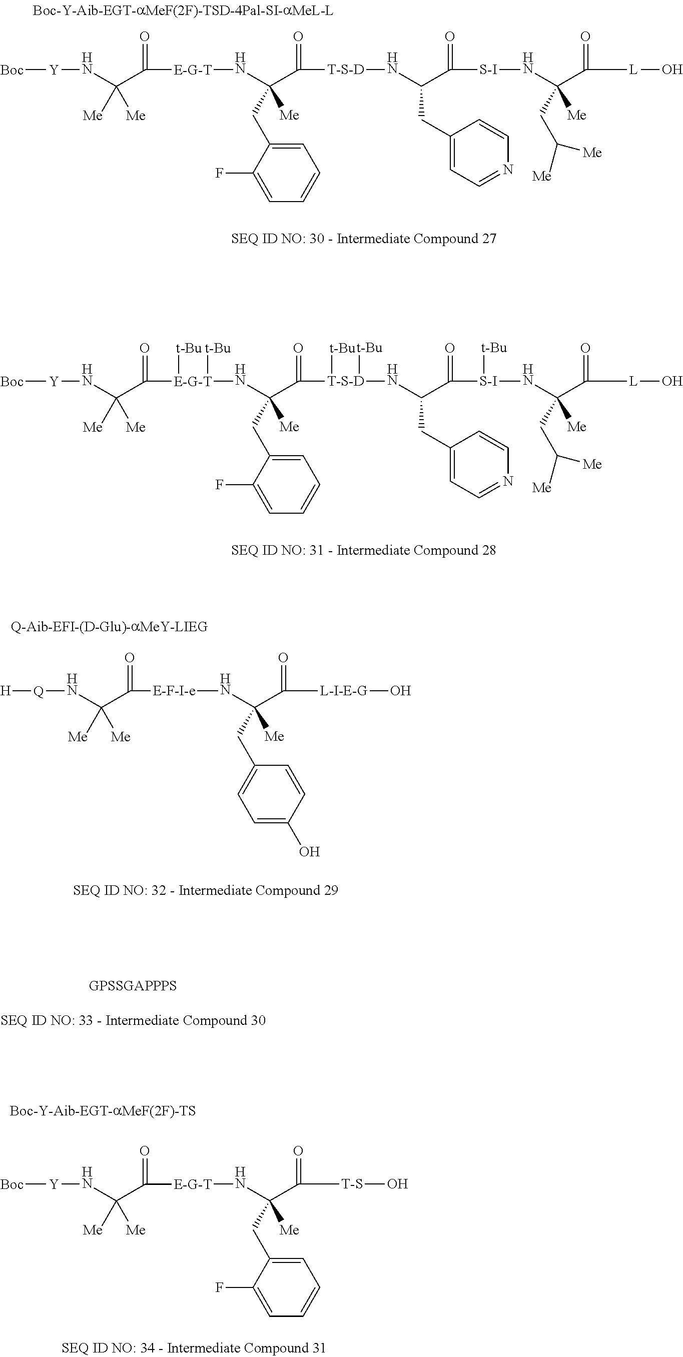

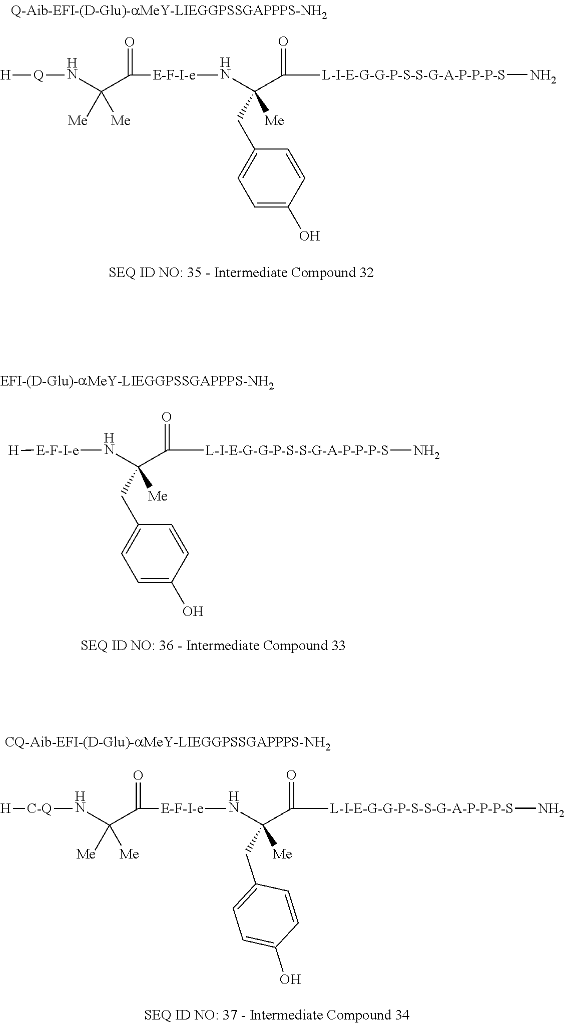

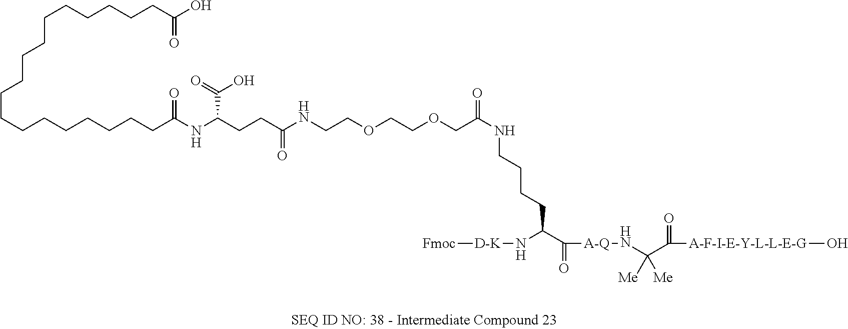

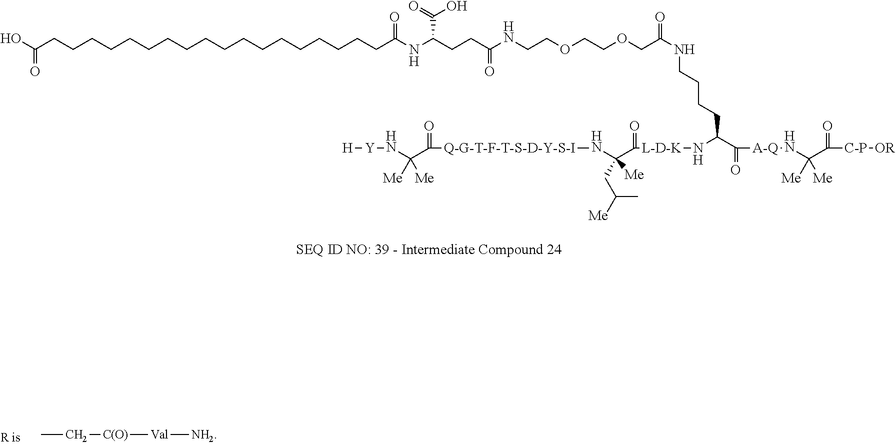

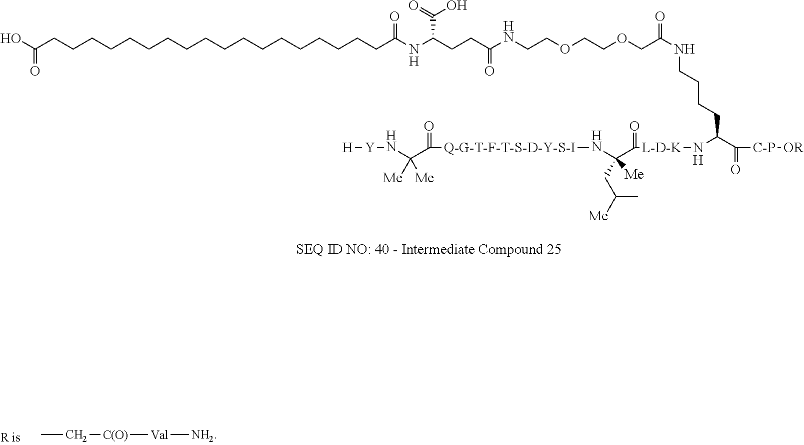

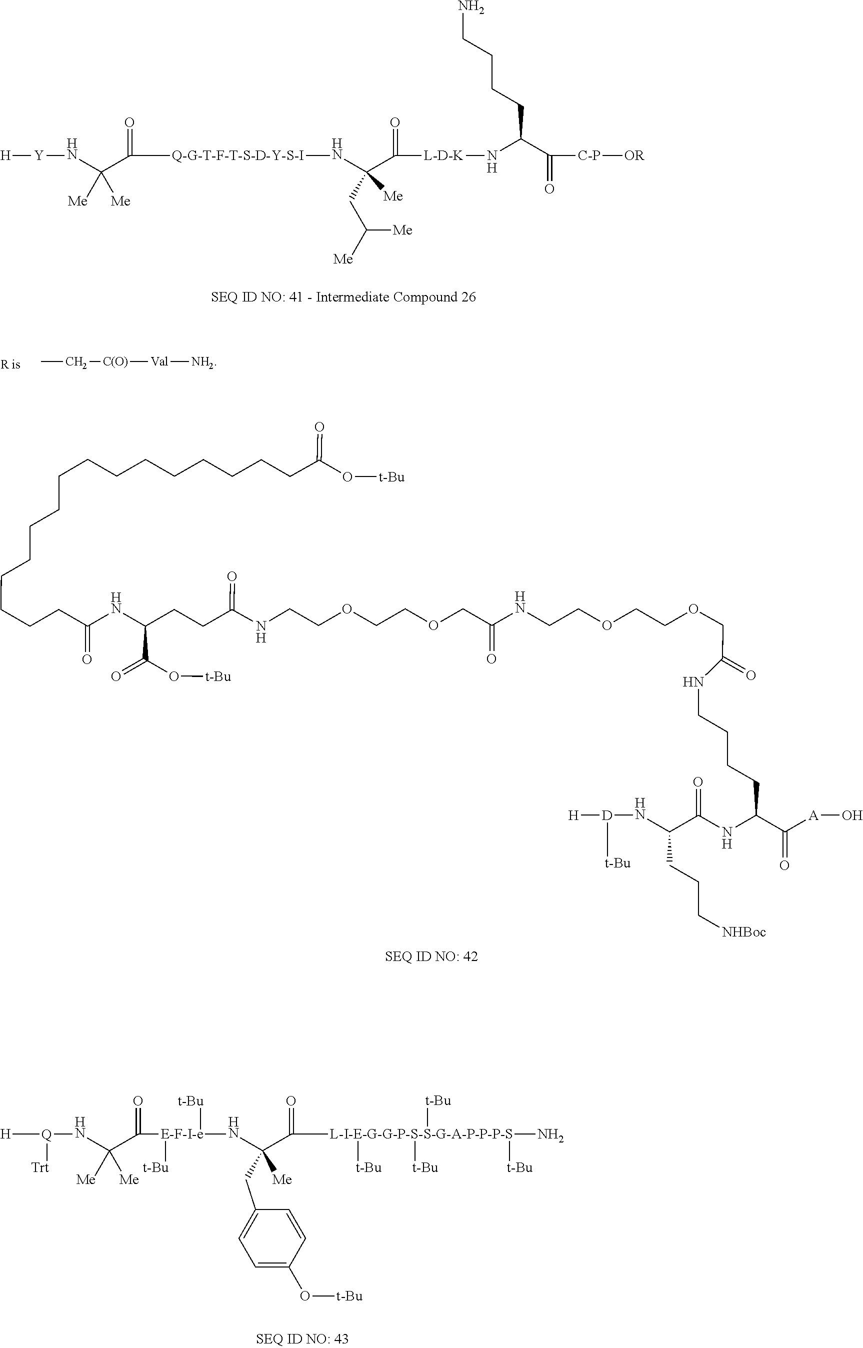

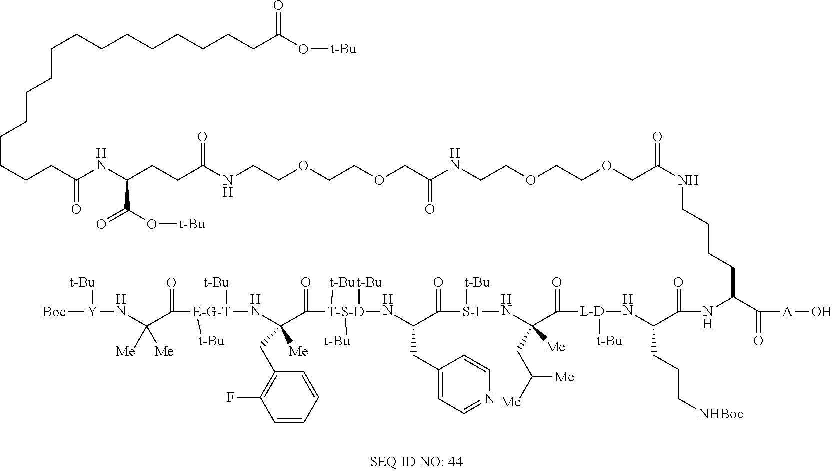

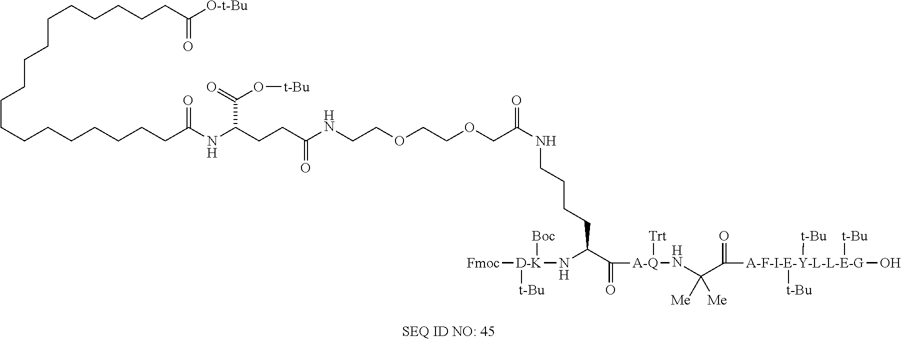

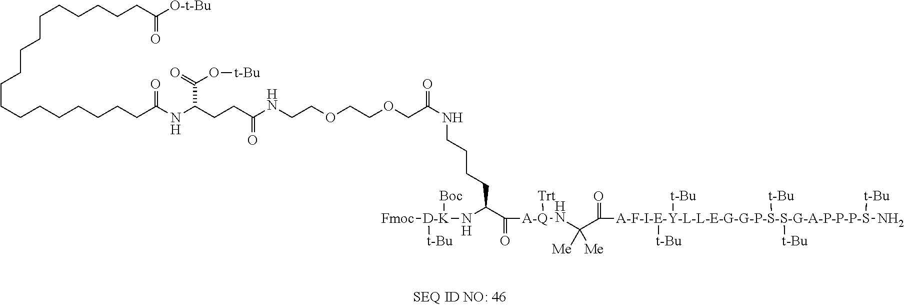

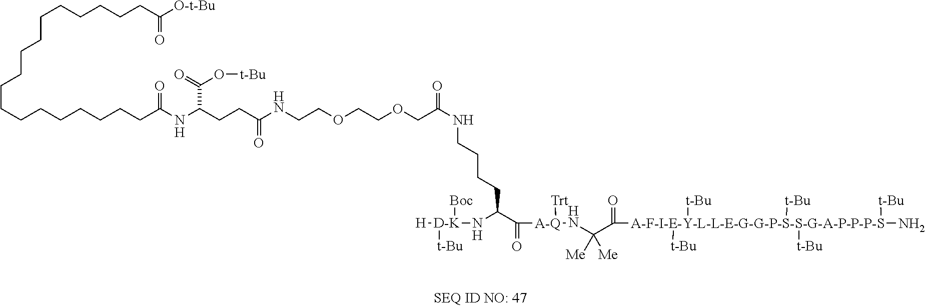

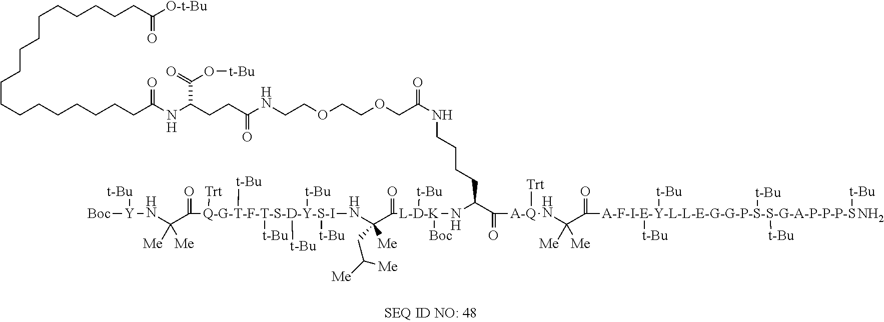

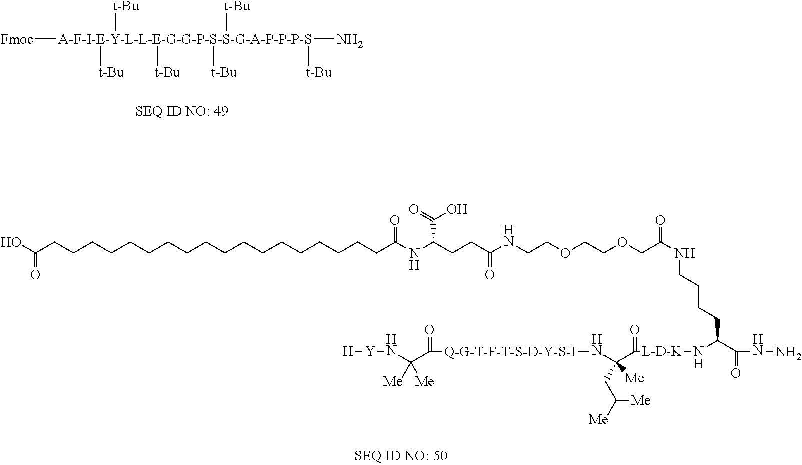

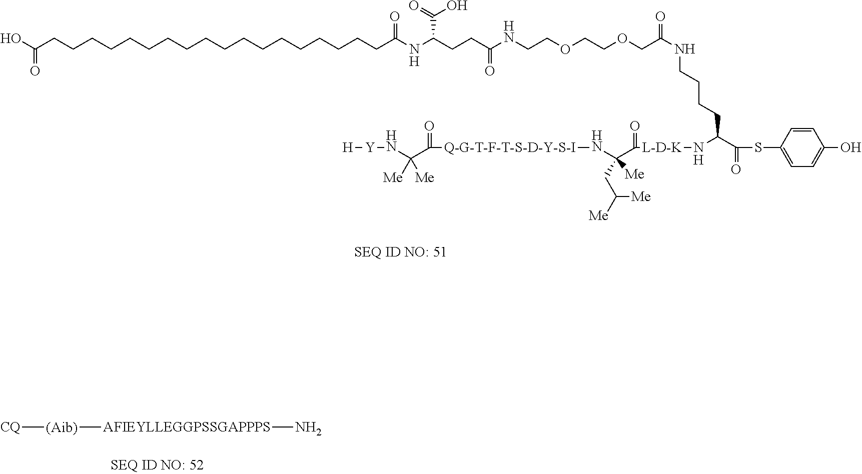

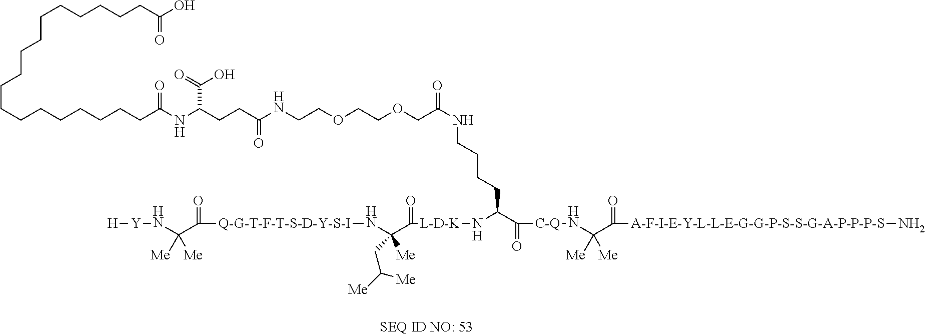

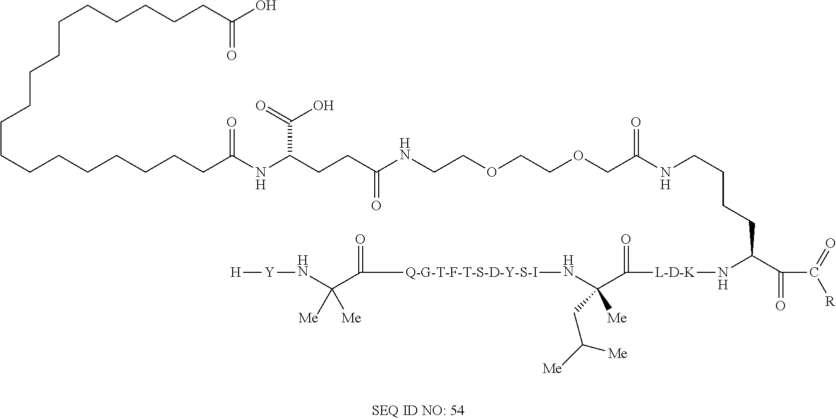

Where R is Pro-glycolic acid-Val or Pro-glycolic acid

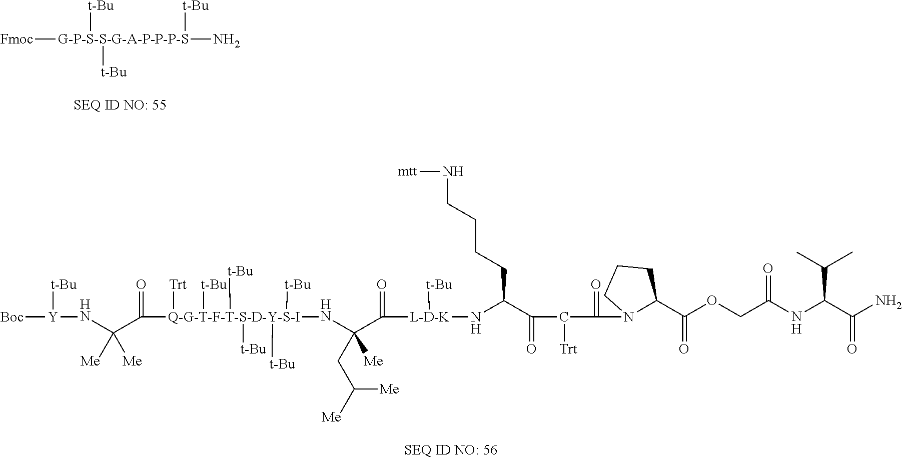

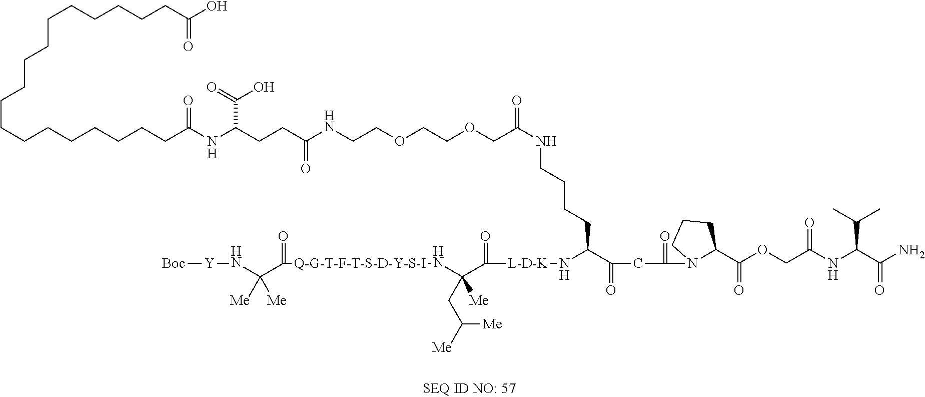

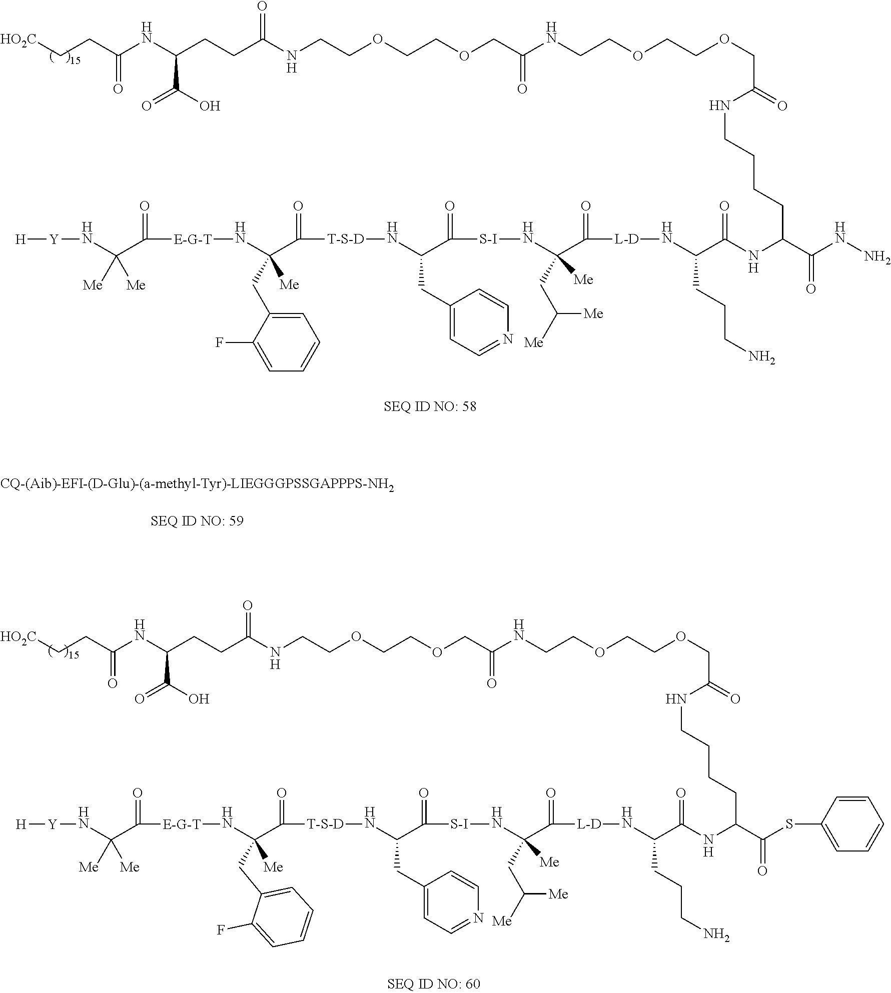

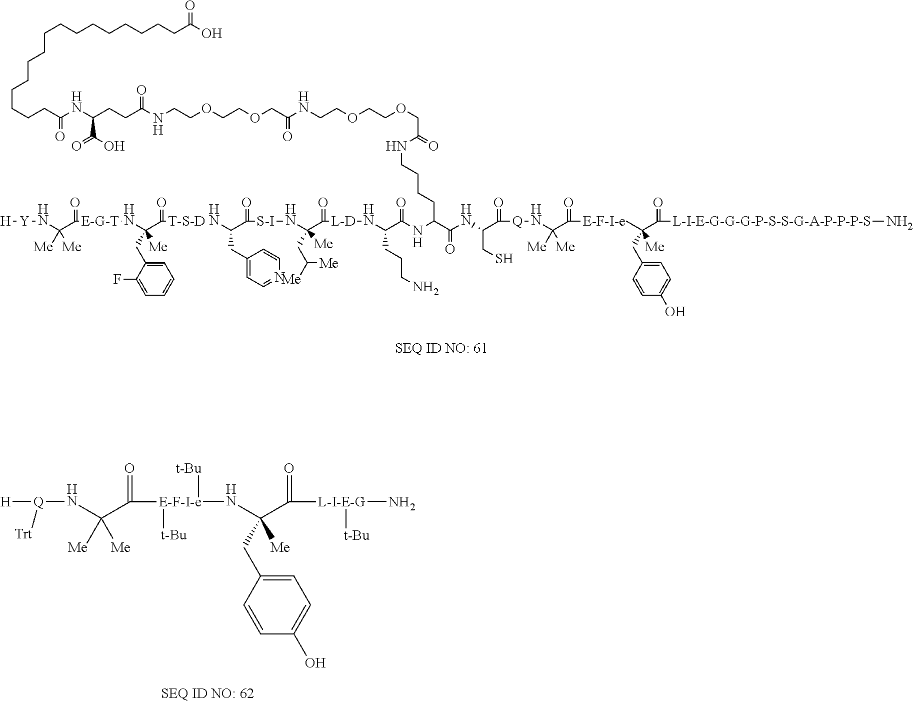

1. A method of making an incretin analog of SEQ ID NO:6, the method comprising the step of:
coupling, via hybrid liquid solid phase synthesis, four intermediate compounds selected from the groups consisting of:
a. SEQ ID NOS:7, 8, 9 and 10,
b. SEQ ID NOS:7, 11, 12, and 10, and
c. SEQ ID NOS:7, 13, 14 and 10.

2. A method of making an incretin analog of SEQ ID NO:6, the method comprising the step of:
coupling, via hybrid liquid solid phase synthesis, three intermediate compounds selected from the groups consisting of:
a. SEQ ID NOS:7, 13 and 15,
b. SEQ ID NOS:16, 17 and 10,
c. SEQ ID NOS:18, 12 and 10, and
d. SEQ ID NOS:7, 45 and 10.

3. A method of making an incretin analog of SEQ ID NO:6, the method comprising the step of:
coupling, via hybrid liquid solid phase synthesis, two intermediate compounds selected from the groups consisting of:
a. SEQ ID NOS:15 and 19 and
b. SEQ ID NOS:18 and 20.

4. An intermediate compound comprising:
any one of SEQ ID NOS:7 to 28, 30 to 54 and 56 to 62, or a pharmaceutically acceptable salt thereof.

5-37. (canceled)

38. A method of making an incretin analog of SEQ ID NO:29, the method comprising the step of:
coupling, via hybrid liquid solid phase synthesis, intermediate compounds selected from the groups consisting of:
a. SEQ ID NOS:7, 62, 42 and 31,
b. SEQ ID NOS:43, and 44.


SEQUENCE LISTING (62 sequences)

SEQ ID NO: 1 - protein, 42 aa, Homo sapiens
YAEGTFISDYSIAMDKIHQQDFVNWLLAQKGKKNDWKHNITQ

SEQ ID NO: 2 - protein, 30 aa, Homo sapiens
  MOD_RES (30)..(30): AMIDATION
HAEGTFTSDVSSYLEGQAAKEFIAWLVKGR

SEQ ID NO: 3 - protein, 29 aa, Homo sapiens
HSQGTFTSDYSKYLDSRRAQDFVQWLMNT

SEQ ID NO: 4 - protein, 39 aa, Artificial Sequence
  Synthetic Construct
  MISC_FEATURE (2)..(2): Xaa at position 2 is Aib
  MISC_FEATURE (13)..(13): Xaa at position 13 is L or alpha-Methyl-L
  MISC_FEATURE (17)..(17): Xaa at position 17 is any amino acid with a functional group available for conjugation, and the functional group is conjugated to a C16-C22 fatty acid
  MISC_FEATURE (19)..(19): Xaa at position 19 is Q or A
  MISC_FEATURE (20)..(20): Xaa at position 20 is Aib, alpha-Methyl-K, Q or H
  MISC_FEATURE (28)..(28): Xaa at position 28 is E or A
  MISC_FEATURE (29)..(29): Xaa at position 29 is G or Aib
  MISC_FEATURE (34)..(34): Xaa at position 34 is G or Aib
  MISC_FEATURE (39)..(39): The C-terminal amino acid is optionally amidated
YXQGTFTSDYSIXLDKXAXXAFIEYLLXXGPSSXAPPPS

SEQ ID NO: 5 - protein, 39 aa, Artificial Sequence
  Synthetic Construct
  MISC_FEATURE (2)..(2): Xaa at position 2 is Aib
  MISC_FEATURE (13)..(13): Xaa at position 13 is alpha-Methyl-L
  MISC_FEATURE (20)..(20): Xaa at position 20 is Aib
YXQGTFTSDYSIXLDKKAQXAFIEYLLEGGPSSGAPPPS

SEQ ID NO: 6 - protein, 39 aa, Artificial Sequence; identical to SEQ ID NO: 5
  Synthetic Construct
  MISC_FEATURE (2)..(2): Xaa at position 2 is Aib
  MISC_FEATURE (13)..(13): Xaa at position 13 is alpha-Methyl-L
  MOD_RES (17)..(17): Lys at position 17 is chemically modified through conjugation to the epsilon-amino group of the K side-chain with (2-[2-(2-Amino-ethoxy)-acetyl)-(gamma-Glu)-CO-(CH2)18-CO2H
  MISC_FEATURE (20)..(20): Xaa at position 20 is Aib
  MOD_RES (39)..(39): Ser at position 39 is amidated as a C-terminal primary amide

SEQ ID NO: 7 - protein, 10 aa, Artificial Sequence
  Synthetic Construct
  MOD_RES (3)..(3): The sidechain of serine is protected by t-butyl
  MOD_RES (4)..(4): The sidechain of serine is protected by t-butyl
  MOD_RES (10)..(10): The sidechain of serine is protected by t-butyl
  MOD_RES (10)..(10): Ser at position 10 is amidated as a C-terminal primary amide
GPSSGAPPPS

SEQ ID NO: 8 - protein, 8 aa, Artificial Sequence
  Synthetic Construct
  MOD_RES (1)..(1): N-terminus is protected by Fmoc.
  MOD_RES (3)..(3): The sidechain of glutamic acid is protected by t-butyl.
  MOD_RES (4)..(4): The sidechain of tyrosine is protected by t-butyl.
  MOD_RES (7)..(7): The sidechain of glutamic acid is protected by t-butyl.
FIEYLLEG

SEQ ID NO: 9 - protein, 7 aa, Artificial Sequence
  Synthetic Construct
  MOD_RES (1)..(1): N-terminus is protected by Fmoc.
  MOD_RES (1)..(1): The sidechain of aspartic acid is protected by t-butyl.
  MOD_RES (2)..(2): The sidechain of lysine is protected by Boc.
  MOD_RES (3)..(3): Lys at position 3 is chemically modified through conjugation to the epsilon-amino group of the K side-chain with (2-[2-(2-Amino-ethoxy)-ethoxy]-acetyl)-(gamma-Glu(t-butyl))- CO-(CH2)18-CO2-(t-butyl).
  MOD_RES (5)..(5): The sidechain of glutamine is protected by Trityl (Trt).
  MISC_FEATURE (6)..(6): Xaa at position 6 is Aib
DKKAQXA

SEQ ID NO: 10 - protein, 14 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib
  MOD_RES (3)..(3): The sidechain of glutamine is protected by Trityl (Trt).
  MOD_RES (5)..(5): The sidechain of threonine is protected by t-butyl.
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MOD_RES (10)..(10): The sidechain of tyrosine is protected by t-butyl.
  MOD_RES (11)..(11): The sidechain of serine is protected by t-butyl.
  MISC_FEATURE (13)..(13): Xaa at position 13 is alpha-Methyl-Leu
YXQGTFTSDYSIXL

SEQ ID NO: 11 - protein, 9 aa, Artificial Sequence
  Synthetic Construct
  MOD_RES (1)..(1): N-terminus is protected by Fmoc.
  MOD_RES (4)..(4): The sidechain of glutamic acid is protected by t-butyl.
  MOD_RES (5)..(5): The sidechain of tyrosine is protected by t-butyl.
  MOD_RES (8)..(8): The sidechain of tyrosine is protected by t-butyl.
AFIEYLLEG

SEQ ID NO: 12 - protein, 6 aa, Artificial Sequence
  Synthetic Construct
  MOD_RES (1)..(1): N-terminus is protected by Fmoc.
  MOD_RES (1)..(1): The sidechain of aspartic acid is protected by t-butyl.
  MOD_RES (2)..(2): The sidechain of lysine is protected by Boc.
  MOD_RES (3)..(3): Lys at position 3 is chemically modified through conjugation to the epsilon-amino group of the K side-chain with (2-[2-(2-Amino-ethoxy)-ethoxy]-acetyl)-(gamma- Glu(t-butyl))-CO-(CH2)18-CO2-(t-butyl).
  MOD_RES (5)..(5): The sidechain of glutamine is protected by Trityl (Trt).
  MISC_FEATURE (6)..(6): Xaa at position 6 is Aib
DKKAQX

SEQ ID NO: 13 - protein, 11 aa, Artificial Sequence
  Synthetic Construct
  MOD_RES (1)..(1): N-terminus is protected by Fmoc.
  MOD_RES (1)..(1): The sidechain of glutamine is protected by Trityl (Trt).
  MISC_FEATURE (2)..(2): Xaa at position 2 is Aib
  MOD_RES (6)..(6): The sidechain of glutamic acid is protected by t-butyl.
  MOD_RES (7)..(7): The sidechain of tyrosine is protected by t-butyl.
  MOD_RES (10)..(10): The sidechain of glutamic acid is protected by t-butyl.
QXAFIEYLLEG

SEQ ID NO: 14 - protein, 4 aa, Artificial Sequence
  Synthetic Construct
  MOD_RES (1)..(1): N-terminus is protected by Fmoc.
  MOD_RES (1)..(1): The sidechain of aspartic acid is protected by t-butyl.
  MOD_RES (2)..(2): The sidechain of lysine is protected by Boc.
  MOD_RES (3)..(3): Lys at position 3 is chemically modified through conjugation to the epsilon-amino group of the K side- chain with (2-[2-(2-Amino-ethoxy)-ethoxy]-acetyl)-(gamma- Glu(t-butyl))-CO-(CH2)18-CO2-(t-butyl).
DKKA

SEQ ID NO: 15 - protein, 18 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib
  MOD_RES (3)..(3): The sidechain of glutamine is protected by Trityl (Trt).
  MOD_RES (5)..(5): The sidechain of threonine is protected by t-butyl.
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MOD_RES (10)..(10): The sidechain of tyrosine is protected by t-butyl.
  MOD_RES (11)..(11): The sidechain of serine is protected by t-butyl.
  MISC_FEATURE (13)..(13): Xaa at position 13 is alpha-methyl-Leu
  MOD_RES (15)..(15): The sidechain of aspartic acid is protected by t-butyl.
  MOD_RES (16)..(16): The sidechain of lysine is protected by Boc.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma- Glu(t-butyl))-CO-(CH2)18-CO2-(t-butyl).
YXQGTFTSDYSIXLDKKA

SEQ ID NO: 16 - protein, 18 aa, Artificial Sequence
  Synthetic Construct
  MOD_RES (3)..(3): The sidechain of glutamic acid is protected by t-butyl.
  MOD_RES (4)..(4): The sidechain of tyrosine is protected by t-butyl.
  MOD_RES (7)..(7): The sidechain of glutamic acid is protected by t-butyl.
  MOD_RES (11)..(11): The sidechain of serine is protected by t-butyl.
  MOD_RES (12)..(12): The sidechain of serine is protected by t-butyl.
  MOD_RES (18)..(18): The sidechain of serine is protected by t-butyl.
  MOD_RES (18)..(18): Ser at position 18 is amidated as a C-terminal primary amide
FIEYLLEGGPSSGAPPPS

SEQ ID NO: 17 - protein, 7 aa, Artificial Sequence
  Synthetic Construct
  MOD_RES (1)..(1): N-terminus is protected by Fmoc.
  MOD_RES (1)..(1): The sidechain of aspartic acid is protected by t-butyl.
  MOD_RES (2)..(2): The sidechain of lysine is protected by Boc.
  MOD_RES (3)..(3): Lys at position 3 is chemically modified through conjugation to the epsilon-amino group of the K side-chain with (2-[2-(2-Amino-ethoxy)-ethoxy]-acetyl)-(gamma-Glu(t-butyl))-CO-(C H2)18-CO2-(t-butyl).
  MOD_RES (5)..(5): The sidechain of glutamine is protected by Trityl (Trt).
  MISC_FEATURE (6)..(6): Xaa at position 6 is Aib
DKKAQXA

SEQ ID NO: 18 - protein, 19 aa, Artificial Sequence
  Synthetic Construct
  MOD_RES (4)..(4): The sidechain of glutamic acid is protected by t-butyl.
  MOD_RES (5)..(5): The sidechain of tyrosine is protected by t-butyl.
  MOD_RES (8)..(8): The sidechain of glutamic acid is protected by t-butyl.
  MOD_RES (12)..(12): The sidechain of serine is protected by t-butyl.
  MOD_RES (13)..(13): The sidechain of serine is protected by t-butyl.
  MOD_RES (19)..(19): The sidechain of serine is protected by t-butyl.
  MOD_RES (19)..(19): Ser at position 19 is amidated as a C-terminal primary amide.
AFIEYLLEGGPSSGAPPPS

SEQ ID NO: 19 - protein, 21 aa, Artificial Sequence
  Synthetic Construct
  MOD_RES (1)..(1): The sidechain of glutamine is protected by Trityl (Trt).
  MISC_FEATURE (2)..(2): Xaa at position 2 is Aib.
  MOD_RES (6)..(6): The sidechain of glutamic acid is protected by t-butyl.
  MOD_RES (7)..(7): The sidechain of tyrosine is protected by t-butyl.
  MOD_RES (10)..(10): The sidechain of glutamic acid is protected by t-butyl.
  MOD_RES (14)..(14): The sidechain of serine is protected by t-butyl.
  MOD_RES (15)..(15): The sidechain of serine is protected by t-butyl.
  MOD_RES (21)..(21): The sidechain of serine is protected by t-butyl.
  MOD_RES (21)..(21): Ser at position 21 is amidated as a C-terminal primary amide
QXAFIEYLLEGGPSSGAPPPS

SEQ ID NO: 20 - protein, 20 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib.
  MOD_RES (3)..(3): The sidechain of glutamine is protected by Trityl (Trt).
  MOD_RES (5)..(5): The sidechain of threonine is protected by t-butyl.
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MOD_RES (10)..(10): The sidechain of tyrosine is protected by t-butyl.
  MOD_RES (11)..(11): The sidechain of serine is protected by t-butyl.
  MISC_FEATURE (13)..(13): Xaa at position 13 is alpha-methyl-Leu
  MOD_RES (15)..(15): The sidechain of aspartic acid is protected by t-butyl.
  MOD_RES (16)..(16): The sidechain of lysine is protected by Boc.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma- Glu(t-butyl))-CO-(CH2)18-CO2-(t-butyl).
  MOD_RES (19)..(19): The sidechain of glutamine is protected by Trityl (Trt).
  MISC_FEATURE (20)..(20): Xaa at position 2 is Aib.
YXQGTFTSDYSIXLDKKAQX

SEQ ID NO: 21 - protein, 20 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib.
  MOD_RES (3)..(3): The sidechain of glutamine is protected by Trityl (Trt).
  MOD_RES (5)..(5): The sidechain of threonine is protected by t-butyl.
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MOD_RES (10)..(10): The sidechain of tyrosine is protected by t-butyl.
  MOD_RES (11)..(11): The sidechain of serine is protected by t-butyl.
  MISC_FEATURE (13)..(13): Xaa at position 13 is alpha-methyl-Leu.
  MOD_RES (15)..(15): The sidechain of aspartic acid is protected by t-butyl.
  MOD_RES (16)..(16): The sidechain of lysine is protected by Boc.
  MOD_RES (17)..(17): The sidechain of Lys at position 17 is protected by a protecting group PG.
  MOD_RES (19)..(19): The sidechain of glutamine is protected by Trityl (Trt).
  MISC_FEATURE (20)..(20): Xaa at position 20 is Aib.
YXQGTFTSDYSIXLDKKAQX

SEQ ID NO: 22 - protein, 18 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib.
  MOD_RES (3)..(3): The sidechain of glutamine is protected by Trityl (Trt).
  MOD_RES (5)..(5): The sidechain of threonine is protected by t-butyl.
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MOD_RES (10)..(10): The sidechain of tyrosine is protected by t-butyl.
  MOD_RES (11)..(11): The sidechain of serine is protected by t-butyl.
  MISC_FEATURE (13)..(13): Xaa at position 13 is alpha-methyl-Leu.
  MOD_RES (15)..(15): The sidechain of aspartic acid is protected by t-butyl.
  MOD_RES (16)..(16): The sidechain of lysine is protected by Boc.
  MOD_RES (17)..(17): The sidechain of lysine is protected by a protecting group PG.
YXQGTFTSDYSIXLDKKA

SEQ ID NO: 23 - protein, 20 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MISC_FEATURE (20)..(20): Xaa at position 20 is Aib.
  MOD_RES (20)..(20): The C-terminal hyroxyl is modified with -SR, wherein R can be 2,2,2-trifluoroethyl.
YXQGTFTSDYSIXLDKKAQX

SEQ ID NO: 24 - protein, 19 aa, Artificial Sequence
  Synthetic Construct
  MOD_RES (1)..(1): The side chain of Ala is modified with -SH.
  MOD_RES (19)..(19): Ser at position 19 is amidated as a C-terminal primary amide.
AFIEYLLEGGPSSGAPPPS

SEQ ID NO: 25 - protein, 20 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MISC_FEATURE (20)..(20): Xaa at position 20 is Aib.
  MOD_RES (20)..(20): The C terminal hydroxyl is modified with -SR, wherein R can be 2,2,2-trifluoroethyl.
YXQGTFTSDYSIXLDKKAQX

SEQ ID NO: 26 - protein, 22 aa, Artificial Sequence
  Synthetic Construct
  MOD_RES (1)..(1): The sidechain of Ala is modified with -SH.
  MISC_FEATURE (3)..(3): Xaa at position 3 is Aib.
  MOD_RES (22)..(22): Ser at position 22 is amidated as a C-terminal primary amide.
AQXAFIEYLLEGGPSSGAPPPS

SEQ ID NO: 27 - protein, 17 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): The C-terminal hydroxyl is modified with -SR, wherein R may be 2,2,2-trifluoroethyl.
YXQGTFTSDYSIXLDKK

SEQ ID NO: 28 - protein, 4 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib.
  MOD_RES (3)..(3): The sidechain of glutamine is protected by Trityl (Trt).
YXQG

SEQ ID NO: 29 - protein, 39 aa, Artificial Sequence
  Synthetic Construct
  MISC_FEATURE (2)..(2): Xaa at position 2 is Aib.
  MISC_FEATURE (6)..(6): Xaa at position 6 is alpha-methyl-Phe(2F).
  MISC_FEATURE (10)..(10): Xaa at position 10 is 4Pal..
  MISC_FEATURE (13)..(13): Xaa at position 13 is alpha-methyl-Leu.
  MISC_FEATURE (16)..(16): Xaa at position 16 is Orn.
  MOD_RES (17)..(17): Lys at position 17 is chemically modified through conjugation to the epsilon-amino group of the K side-chain with (2-[2-(2-Amino-ethoxy)-ethoxy]-acetyl)2-(gamma-Glu)-CO-(CH2)16-CO 2H.
  MISC_FEATURE (20)..(20): Xaa at position 20 is Aib.
  MISC_FEATURE (24)..(24): Xaa at position 24 is D-Glu.
  MISC_FEATURE (25)..(25): Xaa at position 25 is alpha-methyl-Tyr.
  MOD_RES (39)..(39): Ser at position 39 is amidated as a C-terminal primary amide.
YXEGTXTSDXSIXLDXKAQXEFIXXLIEGGPSSGAPPPS

SEQ ID NO: 30 - protein, 14 aa, Artificial Sequence
  Synthetic Construct
  MOD_RES (1)..(1): N-terminus is protected by Boc.
  MISC_FEATURE (2)..(2): Xaa at position 2 is Aib.
  MISC_FEATURE (6)..(6): Xaa at position 6 is alpha-mehtyl-Phe(2F).
  MISC_FEATURE (10)..(10): Xaa at position 10 is 4Pal.
  MISC_FEATURE (13)..(13): Xaa at position 13 is alpha-methyl-Leu.
YXEGTXTSDXSIXL

SEQ ID NO: 31 - protein, 14 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib.
  MOD_RES (3)..(3): The sidechain of glutamic acid is protected by t-butyl.
  MOD_RES (5)..(5): The sidechain of threonine is protected by t-butyl.
  MISC_FEATURE (6)..(6): Xaa at position 6 is alpha-methyl-Phe(2F).
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MISC_FEATURE (10)..(10): Xaa at position 10 is 4Pal.
  MOD_RES (11)..(11): The sidechain of serine is protected by t-butyl.
  MISC_FEATURE (13)..(13): Xaa at position 13 is alpha-methyl-Leu.
YXEGTXTSDXSIXL

SEQ ID NO: 32 - protein, 11 aa, Artificial Sequence
  Synthetic Construct
  MISC_FEATURE (2)..(2): Xaa at position 2 is Aib.
  MISC_FEATURE (6)..(6): Xaa at position 6 is D-Glu.
  MISC_FEATURE (7)..(7): Xaa at position 7 is alpha-methyl-Tyr.
QXEFIXXLIEG

SEQ ID NO: 33 - protein, 10 aa, Artificial Sequence
  Synthetic Construct
GPSSGAPPPS

SEQ ID NO: 34 - protein, 8 aa, Artificial Sequence
  Synthetic Construct
  MOD_RES (1)..(1): N-terminus is protected by Boc.
  MISC_FEATURE (2)..(2): Xaa at position 2 is Aib.
  MISC_FEATURE (6)..(6): Xaa at position 6 is alpha-methyl-Phe(2F).
YXEGTXTS

SEQ ID NO: 35 - protein, 21 aa, Artificial Sequence
  Synthetic Construct
  MISC_FEATURE (2)..(2): Xaa at position 2 is Aib.
  MISC_FEATURE (6)..(6): Xaa at position 6 is D-Glu.
  MISC_FEATURE (7)..(7): Xaa at position 7 is alpha-methyl-Tyr.
  MOD_RES (21)..(21): Ser at position 21 is amidated as a C-terminal primary amide.
QXEFIXXLIEGGPSSGAPPPS

SEQ ID NO: 36 - protein, 19 aa, Artificial Sequence
  Synthetic Construct
  MISC_FEATURE (4)..(4): Xaa at position 4 is D-Glu.
  MISC_FEATURE (5)..(5): Xaa at position 5 is alpha-methyl-Tyr.
  MOD_RES (19)..(19): Ser at position 19 is amidated as a C-terminal primary amide.
EFIXXLIEGGPSSGAPPPS

SEQ ID NO: 37 - protein, 22 aa, Artificial Sequence
  Synthetic Construct
  MISC_FEATURE (3)..(3): Xaa at position 3 is Aib.
  MISC_FEATURE (7)..(7): Xaa at position 7 is D-Glu.
  MISC_FEATURE (8)..(8): Xaa at position 8 is alpha-methyl-Tyr.
  MOD_RES (22)..(22): Ser at position 22 is amidated as a C-terminal primary amide.
CQXEFIXXLIEGGPSSGAPPPS

SEQ ID NO: 38 - protein, 15 aa, Artificial Sequence
  Synthetic Construct
  MOD_RES (1)..(1): N-terminus is protected by Fmoc.
  MOD_RES (3)..(3): Lys at position 3 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MISC_FEATURE (6)..(6): Xaa at position 6 is Aib.
DKKAQXAFIEYLLEG

SEQ ID NO: 39 - protein, 22 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MISC_FEATURE (20)..(20): Xaa at position 20 is Aib.
  MOD_RES (22)..(22): The C-terminal is -OR, wherein R is -CH2-C(O)-Val-NH2.
YXQGTFTSDYSIXLDKKAQXCP

SEQ ID NO: 40 - protein, 19 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MOD_RES (19)..(19): The C-terminal is -OR, wherein R is -CH2-C(O)-Val-NH2.
YXQGTFTSDYSIXLDKKCP

SEQ ID NO: 41 - protein, 19 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (19)..(19): The C-terminal is -OR, wherein R is -CH2-C(O)-Val-NH2.
YXQGTFTSDYSIXLDKKCP

SEQ ID NO: 42 - protein, 4 aa, Artificial Sequence
  Synthetic Construct
  MOD_RES (1)..(1): The sidechain of aspartic acid is protected by t-butyl.
  MISC_FEATURE (2)..(2): Xaa at position 2 is Orn.
  MOD_RES (2)..(2): The sidechain of Orn is protected by Boc.
  MOD_RES (3)..(3): Lys at position 3 is chemically modified through conjugation to the epsilon-amino group of the K side-chain with (2-[2-(2-Amino-ethoxy)-ethoxy]-acetyl)2-(gamma-Glu(t-butyl))-CO-( CH2)16-CO2-(t-butyl).
DXKA

SEQ ID NO: 43 - protein, 21 aa, Artificial Sequence
  Synthetic Construct
  MOD_RES (1)..(1): The sidechain of glutamine is protected by Trityl (Trt).
  MISC_FEATURE (2)..(2): Xaa at position 2 is Aib.
  MOD_RES (3)..(3): The sidechain of glutamic acid is protected by t-butyl.
  MISC_FEATURE (6)..(6): Xaa at position 6 is D-Glu.
  MOD_RES (6)..(6): The sidechain of D-glutamic acid is protected by t-butyl.
  MISC_FEATURE (7)..(7): Xaa at position 7 is alpha-methyl-Tyr.
  MISC_FEATURE (7)..(7): The sidechain of alpha-methyl-tyrosine is protected by t-butyl.
  MOD_RES (10)..(10): The sidechain of glutamic acid is protected by t-butyl.
  MOD_RES (14)..(14): The sidechain of serine is protected by t-butyl.
  MOD_RES (15)..(15): The sidechain of serine is protected by t-butyl.
  MOD_RES (21)..(21): The sidechain of serine is protected by t-butyl.
  MOD_RES (21)..(21): Ser at position 21 is amidated as a C-terminal primary amide.
QXEFIXXLIEGGPSSGAPPPS

SEQ ID NO: 44 - protein, 18 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib.
  MOD_RES (3)..(3): The sidechain of glutamic acid is protected by t-butyl.
  MOD_RES (5)..(5): The sidechain of threonine is protected by t-butyl.
  MISC_FEATURE (6)..(6): Xaa at position 6 is alpha-methyl-Phe(2F).
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MISC_FEATURE (10)..(10): Xaa at position 10 is 4Pal.
  MOD_RES (11)..(11): The sidechain of serine is protected by t-butyl.
  MISC_FEATURE (13)..(13): Xaa at position 13 is alpha-methyl-Leu.
  MOD_RES (15)..(15): The sidechain of aspartic acid is protected by t-butyl.
  MISC_FEATURE (16)..(16): Xaa at position 16 is Orn.
  MOD_RES (16)..(16): The sidechain of Orn is protected by Boc.
  MOD_RES (17)..(17): Lys at position 17 is chemically modified through conjugation to the epsilon-amino group of the K side-chain with (2-[2-(2-Amino-ethoxy)-ethoxy]-acetyl)2-(gamma-Glu(t-butyl))-CO-( CH2)16-CO2-(t-butyl).
YXEGTXTSDXSIXLDXKA

SEQ ID NO: 45 - protein, 15 aa, Artificial Sequence
  Synthetic Construct
  MOD_RES (1)..(1): N-terminus is protected by Fmoc.
  MOD_RES (1)..(1): The sidechain of aspartic acid is protected by t-butyl.
  MOD_RES (2)..(2): The sidechain of lysine is protected by Boc.
  MOD_RES (3)..(3): Lys at position 3 is chemically modified through conjugation to the epsilon-amino group of the K side-chain with (2-[2-(2-Amino-ethoxy)-ethoxy]-acetyl)-(gamma-Glu(t-butyl))-CO-(C H2)18-CO2-(t-butyl).
  MOD_RES (5)..(5): The sidechain of glutamine is protected by Trityl (Trt).
  MISC_FEATURE (6)..(6): Xaa at position 6 is Aib.
  MOD_RES (10)..(10): The sidechain of glutamic acid is protected by t-butyl.
  MOD_RES (11)..(11): The sidechain of tyrosine is protected by t-butyl.
  MOD_RES (14)..(14): The sidechain of glutamic acid is protected by t-butyl.
DKKAQXAFIEYLLEG

SEQ ID NO: 46 - protein, 25 aa, Artificial Sequence
  Synthetic Construct
  MOD_RES (1)..(1): N-terminus is protected by Fmoc.
  MOD_RES (1)..(1): The sidechain of aspartic acid is protected by t-butyl.
  MOD_RES (2)..(2): The sidechain of lysine is protected by Boc.
  MOD_RES (3)..(3): Lys at position 3 is chemically modified through conjugation to the epsilon-amino group of the K side-chain with (2-[2-(2-Amino-ethoxy)-ethoxy]-acetyl)-(gamma-Glu(t-butyl))-CO-(C H2)18-CO2-(t-butyl).
  MOD_RES (5)..(5): The sidechain of glutamine is protected by Trityl (Trt).
  MISC_FEATURE (6)..(6): Xaa at position 6 is Aib.
  MOD_RES (10)..(10): The sidechain of glutamic acid is protected by t-butyl.
  MOD_RES (11)..(11): The sidechain of tyrosine is protected by t-butyl.
  MOD_RES (14)..(14): The sidechain of glutamic acid is protected by t-butyl.
  MOD_RES (18)..(18): The sidechain of serine is protected by t-butyl.
  MOD_RES (19)..(19): The sidechain of serine is protected by t-butyl.
  MOD_RES (25)..(25): The sidechain of serine is protected by t-butyl.
  MOD_RES (25)..(25): Ser at position 25 is amidated as a C-terminal primary amide.
DKKAQXAFIEYLLEGGPSSGAPPPS

SEQ ID NO: 47 - protein, 25 aa, Artificial Sequence
  Synthetic Construct
  MOD_RES (1)..(1): The sidechain of aspartic acid is protected by t-butyl.
  MOD_RES (2)..(2): The sidechain of lysine is protected by Boc.
  MOD_RES (3)..(3): Lys at position 3 is chemically modified through conjugation to the epsilon-amino group of the K side-chain with (2-[2-(2-Amino-ethoxy)-ethoxy]-acetyl)-(gamma-Glu(t-butyl))-CO-(C H2)18-CO2-(t-butyl).
  MOD_RES (5)..(5): The sidechain of glutamine is protected by Trityl (Trt).
  MISC_FEATURE (6)..(6): Xaa at position 6 is Aib.
  MOD_RES (10)..(10): The sidechain of glutamic acid is protected by t-butyl.
  MOD_RES (11)..(11): The sidechain of tyrosine is protected by t-butyl.
  MOD_RES (14)..(14): The sidechain of glutamic acid is protected by t-butyl.
  MOD_RES (18)..(18): The sidechain of serine is protected by t-butyl.
  MOD_RES (19)..(19): The sidechain of serine is protected by t-butyl.
  MOD_RES (25)..(25): The sidechain of serine is protected by t-butyl.
  MOD_RES (25)..(25): Ser at position 25 is amidated as a C-terminal primary amide.
DKKAQXAFIEYLLEGGPSSGAPPPS

SEQ ID NO: 48 - protein, 39 aa, Artificial Sequence; identical to SEQ ID NO: 5
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib.
  MOD_RES (3)..(3): The sidechain of glutamine is protected by Trityl (Trt).
  MOD_RES (5)..(5): The sidechain of threonine is protected by t-butyl.
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MOD_RES (10)..(10): The sidechain of tyrosine is protected by t-butyl.
  MOD_RES (11)..(11): The sidechain of serine is protected by t-butyl.
  MISC_FEATURE (13)..(13): Xaa at position 13 is alpha-methyl-Leu.
  MOD_RES (15)..(15): The sidechain of aspartic acid is protected by t-butyl.
  MOD_RES (16)..(16): The sidechain of lysine is protected by Boc.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu(t-butyl))-CO-(C H2)18-CO2-(t-butyl).
  MOD_RES (19)..(19): The sidechain of glutamine is protected by Trityl (Trt).
  MISC_FEATURE (20)..(20): Xaa at position 20 is Aib.
  MOD_RES (24)..(24): The sidechain of glutamic acid is protected by t-butyl.
  MOD_RES (25)..(25): The sidechain of tyrosine is protected by t-butyl.
  MOD_RES (28)..(28): The sidechain of glutamic acid is protected by t-butyl.
  MOD_RES (32)..(32): The sidechain of serine is protected by t-butyl.
  MOD_RES (33)..(33): The sidechain of serine is protected by t-butyl.
  MOD_RES (39)..(39): The sidechain of serine is protected by t-butyl.
  MOD_RES (39)..(39): Ser at position 39 is amidated as a C-terminal primary amide.

SEQ ID NO: 49 - protein, 19 aa, Artificial Sequence
  Synthetic Construct
  MOD_RES (1)..(1): N-terminus is protected by Fmoc.
  MOD_RES (4)..(4): The sidechain of glutamic acid is protected by t-butyl.
  MOD_RES (5)..(5): The sidechain of tyrosine is protected by t-butyl.
  MOD_RES (8)..(8): The sidechain of glutamic acid is protected by t-butyl.
  MOD_RES (12)..(12): The sidechain of serine is protected by t-butyl.
  MOD_RES (13)..(13): The sidechain of serine is protected by t-butyl.
  MOD_RES (19)..(19): The sidechain of serine is protected by t-butyl.
  MOD_RES (19)..(19): Ser at position 19 is amidated as a C-terminal primary amide.
AFIEYLLEGGPSSGAPPPS

SEQ ID NO: 50 - protein, 17 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MOD_RES (17)..(17): The C-terminus is modified with N-NH2.
YXQGTFTSDYSIXLDKK

SEQ ID NO: 51 - protein, 17 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MOD_RES (17)..(17): The C-terminus is modified with S-(4-OH- phenyl).
YXQGTFTSDYSIXLDKK

SEQ ID NO: 52 - protein, 22 aa, Artificial Sequence
  Synthetic Construct
  MISC_FEATURE (3)..(3): Xaa at position 3 is Aib.
  MOD_RES (22)..(22): Ser at position 22 is amidated as a C-terminal primary amide.
CQXAFIEYLLEGGPSSGAPPPS

SEQ ID NO: 53 - protein, 39 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MISC_FEATURE (20)..(20): Xaa at position 20 is Aib.
  MOD_RES (39)..(39): Ser at position 39 is amidated as a C-terminal primary amide.
YXQGTFTSDYSIXLDKKCQXAFIEYLLEGGPSSGAPPPS

SEQ ID NO: 54 - protein, 18 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MOD_RES (18)..(18): The C-terminus is modified with -(CO)-R, wherein R is Pro-(glycolic acid)-Val or Pro-(glycolic acid).
YXQGTFTSDYSIXLDKKC

SEQ ID NO: 55 - protein, 10 aa, Artificial Sequence
  Synthetic Construct
  MOD_RES (1)..(1): N-terminus is protected by Fmoc.
  MOD_RES (3)..(3): The sidechain of serine is protected by t-butyl.
  MOD_RES (4)..(4): The sidechain of serine is protected by t-butyl.
  MOD_RES (10)..(10): The sidechain of serine is protected by t-butyl.
  MOD_RES (10)..(10): Ser at position 10 is amidated as a C-terminal primary amide.
GPSSGAPPPS

SEQ ID NO: 56 - protein, 18 aa, Artificial Sequence
  Synthetic Construct
  MOD_RES (1)..(1): N-terminus is protected by Boc.
  MOD_RES (1)..(1): The sidechain of tyrosine is protected by t-butyl.
  MISC_FEATURE (2)..(2): Xaa at position 2 is Aib.
  MOD_RES (3)..(3): The sidechain of glutamine is protected by Trityl (Trt).
  MOD_RES (5)..(5): The sidechain of threonine is protected by t-butyl.
  MOD_RES (7)..(7): The sidechain of threonine is protected by t-butyl.
  MOD_RES (8)..(8): The sidechain of serine is protected by t-butyl.
  MOD_RES (9)..(9): The sidechain of aspartic acid is protected by t-butyl.
  MOD_RES (10)..(10): The sidechain of tyrosine is protected by t-butyl.
  MOD_RES (11)..(11): The sidechain of serine is protected by t-butyl.
  MISC_FEATURE (13)..(13): Xaa at position 13 is alpha-methyl-Leu.
  MOD_RES (15)..(15): The sidechain of aspartic acid is protected by t-butyl.
  MOD_RES (17)..(17): The sidechain of lysine is modified with mtt.
  MOD_RES (18)..(18): The sidechain of cysteine is protected by t-butyl.
  MOD_RES (18)..(18): The C-terminus is modified with Pro-(glycolic acid)-Val and Val is amidated.
YXQGTFTSDYSIXLDKKC

SEQ ID NO: 57 - protein, 18 aa, Artificial Sequence
  Synthetic Construct
  MISC_FEATURE (2)..(2): Xaa at position 2 is Aib.
  MISC_FEATURE (13)..(13): Xaa at position 13 is alpha-methyl-Leu.
  MOD_RES (17)..(17): Lys at position 17 is chemically modified through conjugation to the epsilon-amino group of the K side-chain with (2-[2-(2-Amino-ethoxy)-ethoxy]-acetyl)-(gamma-Glu)-CO-(CH2)18-CO2 H.
  MOD_RES (18)..(18): The C-terminus is modified with -Pro-(glycolic acid)-Val, and Val is amidated.
YXQGTFTSDYSIXLDKKC

SEQ ID NO: 58 - protein, 17 aa, Artificial Sequence
  Synthetic Construct
  MISC_FEATURE (2)..(2): Xaa at position 2 is Aib.
  MISC_FEATURE (6)..(6): Xaa at position 6 is alpha-methyl-Phe(2F).
  MISC_FEATURE (10)..(10): Xaa at position 10 is 4Pal.
  MISC_FEATURE (13)..(13): Xaa at position 13 is alpha-methyl-Leu.
  MISC_FEATURE (16)..(16): Xaa at position 16 is Orn.
  MOD_RES (17)..(17): Lys at position 17 is chemically modified through conjugation to the epsilon-amino group of the K side-chain with (2-[2-(2-Amino-ethoxy)-ethoxy]-acetyl)2-(gamma-Glu)-CO-(CH2)16-CO 2H.
  MOD_RES (17)..(17): The C-terminus is modified with N-NH2.
YXEGTXTSDXSIXLDXK

SEQ ID NO: 59 - protein, 23 aa, Artificial Sequence
  Synthetic Construct
  MISC_FEATURE (3)..(3): Xaa at position 3 is Aib.
  MISC_FEATURE (7)..(7): Xaa at position 7 is D-Glu.
  MISC_FEATURE (8)..(8): Xaa at position 8 is alpha-methyl-Tyr.
  MOD_RES (23)..(23): Ser at position 23 is amidated as a C-terminal primary amide.
CQXEFIXXLIEGGGPSSGAPPPS

SEQ ID NO: 60 - protein, 17 aa, Artificial Sequence
  Synthetic Construct
  MISC_FEATURE (2)..(2): Xaa at position 2 is Aib.
  MISC_FEATURE (6)..(6): Xaa at position 6 is alpha-methyl-Phe(2F).
  MISC_FEATURE (10)..(10): Xaa at position 10 is 4Pal.
  MISC_FEATURE (13)..(13): Xaa at position 13 is alpha-methyl-Leu.
  MISC_FEATURE (16)..(16): Xaa at position 16 is Orn.
  MOD_RES (17)..(17): Lys at position 17 is chemically modified through conjugation to the epsilon-amino group of the K side-chain with (2-[2-(2-Amino-ethoxy)-ethoxy]-acetyl)2-(gamma-Glu)-CO-(CH2)16-CO 2H.
  MOD_RES (17)..(17): The C-terminus is modified with -S-phenyl.
YXEGTXTSDXSIXLDXK

SEQ ID NO: 61 - protein, 40 aa, Artificial Sequence
  Synthetic Construct
  MISC_FEATURE (2)..(2): Xaa at position 2 is Aib.
  MISC_FEATURE (6)..(6): Xaa at position 6 is alpha-methyl-Phe(2F).
  MISC_FEATURE (10)..(10): Xaa at position 10 is 4Pal.
  MISC_FEATURE (13)..(13): Xaa at position 13 is alpha-methyl-Leu.
  MISC_FEATURE (16)..(16): Xaa at position 16 is Orn.
  MOD_RES (17)..(17): Lys at position 17 is chemically modified through conjugation to the epsilon-amino group of the K side-chain with (2-[2-(2-Amino-ethoxy)-ethoxy]-acetyl)2-(gamma- Glu)-CO-(CH2)16-CO2H.
  MISC_FEATURE (20)..(20): Xaa at position 20 is Aib.
  MISC_FEATURE (24)..(24): Xaa at position 24 is D-Glu.
  MISC_FEATURE (25)..(25): Xaa at position 25 is alpha-methyl-Tyr.
  MOD_RES (39)..(39): Ser at position 39 is amidated as a C-terminal primary amide.
YXEGTXTSDXSIXLDXKCQXEFIXXLIEGGGPSSGAPPPS

SEQ ID NO: 62 - protein, 11 aa, Artificial Sequence
  Synthetic Construct
  MOD_RES (1)..(1): The sidechain of glutamine is protected by Trityl (Trt).
  MISC_FEATURE (2)..(2): Xaa at position 2 is Aib.
  MOD_RES (3)..(3): The sidechain of glutamic acid is protected by t-butyl.
  MISC_FEATURE (6)..(6): Xaa at position 6 is D-Glu.
  MOD_RES (6)..(6): The sidechain of D-Glu is protected by t-butyl.
  MISC_FEATURE (7)..(7): Xaa at position 7 is alpha-methyl-Tyr.
  MOD_RES (7)..(7): The sidechain of alpha-methyl-tyrosine is protected by t-butyl.
  MOD_RES (10)..(10): The sidechain of glutamic acid is protected by t-butyl.
QXEFIXXLIEG